(19) United States
(12) Patent Application Publication
(10) Pub. No.: US 2026/0063643 A1
(43) Pub. Date: Mar. 5, 2026

(54) ANTI-DRUG ANTIBODY ASSAYS

(71) Applicant: AMGEN INC., Thousand Oaks, CA (US)

(72) Inventors: Mieke Jill MILLER, Thousand Oaks, CA (US); Chadwick T. KING, North Vancouver (CA); Mark Andrew KROENKE, Newbury Park, CA (US); Marta STARCEVIC MANNING, Thousand Oaks, CA (US)

(73) Assignee: AMGEN INC., Thousand Oaks, CA (US)

(21) Appl. No.: 19/105,474

(22) PCT Filed: Aug. 23, 2023
(86) PCT No.: PCT/US2023/072710

§ 371 Date: Feb. 21, 2025

Related U.S. Application Data
(60) Provisional application No. 63/373,426, filed on Aug. 24, 2022.

(51) Int. Cl. G01N 33/68; C07K 16/28

(52) U.S. Cl. CPC G01N 33/6854; C07K 16/2875; C07K 2317/52; C07K 2317/565; C07K 2317/76; G01N 2333/70575

(57) ABSTRACT

The present invention relates methods of detecting anti-drug antibodies and kits for using such assays.

### RELATED APPLICATIONS

This application claims the benefit under 35 U.S.C. § 119(e) to U.S. Provisional Application No. 63/373,426, filed Aug. 24, 2022, which is incorporated herein by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 280,159 byte XML Document named “10200-US01-PRI_Sequence_listing”; created on Aug. 24, 2022.

### FIELD OF THE INVENTION

The present invention relates methods of detecting anti-drug antibodies and kits for using such assays.

### BACKGROUND

Assays for the detection of anti-drug antibodies (ADA) facilitate understanding of potential immune responses to biologic drug candidates. Determining the presence of ADAs and evaluating their clinical implications are a necessary part of any large molecule development program.

All biopharmaceuticals are potentially immunogenic and may induce ADAs. The clinical effects of ADA formation can be highly variable and may cause severe adverse events that put a patient at risk. It is therefore important, both to guide drug development decision-making and as a regulatory requirement, to develop and validate ADA assays with the appropriate sensitivity, specificity, and selectivity for detection. Since ADA responses can affect the pharmacokinetics (PK), pharmacodynamics (PD), safety, and efficacy of a therapeutic candidate, ADA assays should be designed to detect antibodies that could mediate hypersensitivity responses or that have the ability to interfere with interactions between the therapeutic and its target for a neutralizing effect.

In 2019, the FDA issued a Guidance for Industry titled “Immunogenicity Testing of Therapeutic Protein Products—Developing and Validating Assays for Anti-Drug Antibody Detection.” The guidance provides recommendations to facilitate industry's development and validation of assays for assessment of the immunogenicity of therapeutic protein products during clinical trials.

There is a need to develop ADA assays that address accuracy, target interference, and at the same time, meet the 2019 FDA guidance.

### SUMMARY

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).

E1. A method of detecting an anti-drug antibody (ADA) in a sample, wherein said ADA binds to denosumab, comprising mixing said sample with:
(a) a first antibody or antibody conjugate that binds to RANKL, comprising: the heavy chain (HC) CDR-H1, CDR-H2, and CDR-H3 of SEQ ID NO:203, and the light chain (LC) CDR-L1, CDR-L2, and CDR-L3 of SEQ ID NO:208; wherein said first antibody or antibody conjugate can be attached to a solid substrate;
(b) (i) a second antibody that binds to RANKL, wherein said second antibody comprises an amino acid sequence that differs from the first antibody by at least one amino acid in one of the six CDR sequences; or (ii) an osteoprotegerin (OPG) protein that binds to RANKL; and
(c) a third antibody that binds to said ADA and can be linked to a detectable marker.
   E2. The method of E1, wherein said sample comprises soluble RANKL that interferes with the detection of said ADA.
   E3. A method of reducing false signals of an anti-drug antibody (ADA) assay, wherein said ADA assay is to detect the presence of an anti-denosumab antibody in a sample, comprising mixing said sample with:
(a) a first antibody or antibody conjugate that binds to RANKL, comprising: the heavy chain (HC) CDR-H1, CDR-H2, and CDR-H3 of SEQ ID NO:203, and the light chain (LC) CDR-L1, CDR-L2, and CDR-L3 of SEQ ID NO:208; wherein said first antibody or antibody conjugate can be attached to a solid substrate;
(b) (i) a second antibody that binds to RANKL, wherein said second antibody comprises an amino acid sequence that differs from the first antibody by at least one amino acid in one of the six CDR sequences; or (ii) an osteoprotegerin (OPG) protein that binds to RANKL; and
(c) a third antibody that binds to said ADA and can be linked to a detectable marker.
   E4. The method of E3, wherein said false signals are caused by the presence of soluble RANKL that interferes with the detection of said ADA.
   E5. The method of any one of E1-E4, wherein said first antibody conjugate comprises an antibody molecule covalently linked to an immobilizing moiety.
   E6. The method of any one of E1-E4, wherein said first antibody conjugate comprises an antibody molecule noncovalently linked to an immobilizing moiety.
   E7. The method of E5 or E6, wherein said solid substrate comprises a binding partner, and said immobilizing moiety can be attached to a solid substrate by binding to said binding partner.
   E8. The method of E7, wherein said immobilizing moiety is biotin and said binding partner is streptavidin or avidin; or said immobilizing moiety is avidin or streptavidin and said binding partner is biotin.
   E9. The method of E7, wherein said immobilizing moiety and said binding partner are one of the following binding pairs: an antigen and an antibody that binds to said antigen; a lectin and a polysaccharide, a steroid and a steroid binding protein, a hormone and a hormone receptor, an enzyme and a substrate of the enzyme, an IgG and Protein A, an IgG and Protein G; or a ligand and a receptor for the ligand.
   E10. The method of any one of E1-E4, wherein said first antibody is immobilized to a solid substrate that comprises Protein A or Protein G.
   E11. The method of any one E1-E10, wherein said first antibody or antibody conjugate comprise a CDR-H1 comprising SEQ ID NO:200, a CDR-H2 comprising SEQ ID NO:201, and a CDR-H3 comprising SEQ ID NO:202; and a CDR-L1 comprising SEQ ID NO:205, a CDR-L2 comprising SEQ ID NO:206, and a CDR-L3 comprising SEQ ID NO:207.
   E12. The method of any one of E1-E11, wherein said first antibody or antibody conjugate comprises a heavy chain variable region (VH) that is at least 90%, at least 91%, at least 92%, at least 93%, at least 94%, at least 95%, at least 96%, at least 97%, at least 98%, at least 99%, or 100% identical to SEQ ID NO:203.
   E13. The method of any one of E1-E12, wherein said first antibody or antibody conjugate comprises a light chain variable region (VL) that is at least 90%, at least 91%, at least 92%, at least 93%, at least 94%, at least 95%, at least 96%, at least 97%, at least 98%, at least 99%, or 100% identical to SEQ ID NO:208.
   E14. The method of any one of E1-E13, wherein said first antibody or antibody conjugate comprises a human CH1 domain, such as a human IgG1 CH1, a human IgG2 CH1, a human IgG3 CH1, or a human IgG4 CH1.
   E15. The method of E14, wherein said human IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:182.
   E16. The method of E14, wherein:
said human IgG2 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:194; said human IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:190; or
said human IgG4 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:186.
   E17. The method of any one of E1-E16, wherein said first antibody or antibody conjugate comprises a human Fc domain, such as a human IgG1 Fc, a human IgG2 Fc, a human IgG3 Fc, or a human IgG4 Fc.
   E18. The method of E17, wherein said human IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:184.
   E19. The method of E17, wherein:
said human IgG2 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:196;
said human IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:192; or
said human IgG4 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:188.
   E20. The method of any one of E1-E13, wherein said first antibody or antibody conjugate comprises a murine CH1 domain, such as a murine IgG1 CH1, a murine IgG2a CH1, a murine IgG2b CH1, or a murine IgG3 CH1.
   E21. The method of E20, wherein said murine IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:172.
   E22. The method of E20, wherein said murine IgG2a CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:175.
   E23. The method of E20, wherein said murine IgG2b CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:176.
   E24. The method of E20, wherein said murine IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:177.
   E25. The method of any one of E1-E13 and E20-E24, wherein said first antibody or antibody conjugate comprises a murine Fc domain, such as a murine IgG1 Fc, a murine IgG2a Fc, a murine IgG2b Fc, or a murine IgG3 Fc.
   E26. The method of E25, wherein said murine IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:174.
   E27. The method of E25, wherein said murine IgG2a Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:175.
   E28. The method of E25, wherein said murine IgG2b Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:176.
   E29. The method of E25, wherein said murine IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:177.
   E30. The method of any one of E1-E29, wherein said first antibody or antibody conjugate comprises a human CL domain, such as a human kappa CL, or a human lambda CL.
   E31. The method of E30, wherein said human kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:178 or 179.
   E32. The method of E30, wherein said human lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:180 or 181.
   E33. The method of any one of E1-E29, wherein said first antibody or antibody conjugate comprises a murine CL domain, such as a murine kappa CL, or a murine lambda CL.
   E34. The method of E33, wherein said murine kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:171.
   E35. The method of E33, wherein said murine lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:169 or 170.
   E36. The method of any one of E1-E35, wherein said third antibody is covalently linked to a detectable marker.
   E37. The method of any one of E1-E35, wherein said third antibody is noncovalently linked to a detectable marker.
   E38. The method of any one of E1-E37, wherein said detectable marker is an isotope, an enzyme, a fluorescent moiety, a luminescent moiety, a chromatic moiety, a metal, or an electric charge.
   E39. The method of any one of E1-E38, wherein said third antibody comprises six CDR sequences that are identical to that of the first antibody.
   E40. The method of any one of E1-E39, wherein said third antibody comprise:
(i) the heavy chain (HC) CDR-H1, CDR-H2, and CDR-H3 of SEQ ID NO:203, and the light chain (LC) CDR-L1, CDR-L2, and CDR-L3 of SEQ ID NO:208; or
(ii) a CDR-H1 comprising SEQ ID NO:200, a CDR-H2 comprising SEQ ID NO:201, and a CDR-H3 comprising SEQ ID NO:202; and a CDR-L1 comprising SEQ ID NO:205, a CDR-L2 comprising SEQ ID NO:206, and a CDR-L3 comprising SEQ ID NO:207.
   E41. The method of any one of E1-E40, wherein said third antibody comprises a heavy chain variable region (VH) that is at least 90%, at least 91%, at least 92%, at least 93%, at least 94%, at least 95%, at least 96%, at least 97%, at least 98%, at least 99%, or 100% identical to SEQ ID NO:203.
   E42. The method of any one of E1-E41, wherein said third antibody comprises a light chain variable region (VL) that is at least 90%, at least 91%, at least 92%, at least 93%, at least 94%, at least 95%, at least 96%, at least 97%, at least 98%, at least 99%, or 100% identical to SEQ ID NO:208.
   E43. The method of any one of E1-E42, wherein said third antibody comprises a human CH1 domain, such as a human IgG1 CH1, a human IgG2 CH1, a human IgG3 CH1, or a human IgG4 CH1.
   E44. The method of E43, wherein said human IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:182.
   E45. The method of E43, wherein:
said human IgG2 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:194;
said human IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:190; or
said human IgG4 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:186.
   E46. The method of any one of E1-E45, wherein said third antibody comprises a human Fc domain, such as a human IgG1 Fc, a human IgG2 Fc, a human IgG3 Fc, or a human IgG4 Fc.
   E47. The method of E46, wherein said human IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:184.
   E48. The method of E46, wherein
said human IgG2 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:196;
said human IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:192; or
said human IgG4 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:188.
   E49. The method of any one of E1-E42, wherein said third antibody comprises a murine CH1 domain, such as a murine IgG1 CH1, a murine IgG2a CH1, a murine IgG2b CH1, ora murine IgG3 CH1.
   E50. The method of E49, wherein said murine IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:172.
   E51. The method of E49, wherein said murine IgG2a CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:175.
   E52. The method of E49, wherein said murine IgG2b CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:176.
   E53. The method of E49, wherein said murine IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:177.
   E54. The method of any one of E1-E42 and E49-E53, wherein said third antibody comprises a murine Fc domain, such as a murine IgG1 Fc, a murine IgG2a Fc, a murine IgG2b Fc, or a murine IgG3 Fc.
   E55. The method of E54, wherein said murine IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:174.
   E56. The method of E54, wherein said murine IgG2a Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:175.
   E57. The method of E54, wherein said murine IgG2b Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:176.
   E58. The method of E54, wherein said murine IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:177.
   E59. The method of any one of E1-E58, wherein said third antibody comprises a human CL domain, such as a human kappa CL, or a human lambda CL.
   E60. The method of E59, wherein said human kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:178 or 179.
   E61. The method of E59, wherein said human lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:180 or 181.
   E62. The method of any one of E1-E58, wherein said third antibody comprises a murine CL domain, such as a murine kappa CL, or a murine lambda CL.
   E63. The method of E62, wherein said murine kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:171.
   E64. The method of E62, wherein said murine lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:169 or 170.
   E65. The method of any one of E1-E64, wherein said third antibody comprises a VH domain comprising the same sequence as the VH of the first antibody, and a VL domain comprising the same sequence as the VL domain of the first antibody.
   E66. The method of any one of E1-E65, wherein said third antibody comprises a heavy chain (HC) comprising the same sequence as the HC of the first antibody, and a light chain (LC) comprising the same sequence as the LC of the first antibody.
   E67. The method of any one of E1-E66, wherein said detectable marker comprises a secondary antibody that binds to a constant domain of said third antibody.
   E68. The method of any one of E1-E67, wherein said detectable marker comprises a secondary antibody that binds to the Fc domain of said third antibody.
   E69. The method of any one of E1-E66, wherein said third antibody is covalently linked to a fluorescent moiety, a luminescent moiety, or a chromatic moiety.
   E70. The method of any one of E1-E69, wherein said second antibody binds to the same epitope as denosumab.
   E71. The method of any one of E1-E70, wherein said second antibody competes with denosumab for RANKL binding.
   E72. The method of any one of E1-E71, wherein said second antibody comprises an amino acid sequence that differs from the first antibody by at least one amino acid in each of the six CDR sequences.
   E73. The method of any one of E1-E72, wherein said second antibody comprises an amino acid sequence that differs from the first antibody by at least two, at least three, at least four, at least five, at least six amino acids in CDR sequences.
   E74. The method of any one of E1-E73, wherein said ADA does not bind to said second antibody.
   E75. The method of any one of E1-E74, wherein said second antibody comprises: (i) the heavy chain CDR-H1, CDR-H2, and CDR-H3 of SEQ ID NO: 127, 129, 131, 133, 135, 137, 139, 141, 143, 145, 147, 149, 151, 153, 155, 157, 159, 161, 163, 165, or 167; and (ii) the light chain CDR-L1, CDR-L2, and CDR-L3 of SEQ ID NO: 128, 130, 132, 134, 136, 138, 140, 142, 144, 146, 148, 150, 152, 154, 156, 158, 160, 162, 164, 166, or 168.
   E76. The method of any one of E1-E75, wherein said second antibody comprises:
(i) a CDR-H1 comprising a sequence that is at least 90%, at least 91%, at least 92%, at least 93%, at least 94%, at least 95%, at least 96%, at least 97%, at least 98%, at least 99%, or 100% identical to SEQ ID NO: 1, 7, 13, 19, 25, 31, 37, 43, 49, 55, 61, 67, 73, 79, 85, 91, 97, 103, 109, 115, or 121;
(ii) a CDR-H2 comprising a sequence that is at least 90%, at least 91%, at least 92%, at least 93%, at least 94%, at least 95%, at least 96%, at least 97%, at least 98%, at least 99%, or 100% identical to SEQ ID NO: 2, 8, 14, 20, 26, 32, 38, 44, 50, 56, 62, 68, 74, 80, 86, 92, 98, 104, 110, 116, or 122;
(iii) a CDR-H3 comprising a sequence that is at least 90%, at least 91%, at least 92%, at least 93%, at least 94%, at least 95%, at least 96%, at least 97%, at least 98%, at least 99%, or 100% identical to SEQ ID NO: 3, 9, 15, 21, 27, 33, 39, 45, 51, 57, 63, 69, 75, 81, 87, 93, 99, 105, 111, 117, or 123;
(iv) a CDR-L1 comprising a sequence that is at least 90%, at least 91%, at least 92%, at least 93%, at least 94%, at least 95%, at least 96%, at least 97%, at least 98%, at least 99%, or 100% identical to SEQ ID NO:4, 10, 16, 22, 28, 34, 40, 46, 52, 58, 64, 70, 76, 82, 88, 94, 100, 106, 112, 118, or 124;
(v) a CDR-L2 comprising a sequence that is at least 90%, at least 91%, at least 92%, at least 93%, at least 94%, at least 95%, at least 96%, at least 97%, at least 98%, at least 99%, or 100% identical to SEQ ID NO:5, 11, 17, 23, 29, 35, 41, 47, 53, 59, 65, 71, 77, 83, 89, 95, 101, 107, 113, 119, or 125; and
(vi) a CDR-L3 comprising a sequence that is at least 90%, at least 91%, at least 92%, at least 93%, at least 94%, at least 95%, at least 96%, at least 97%, at least 98%, at least 99%, or 100% identical to SEQ ID NO:6, 12, 18, 24, 30, 36, 42, 48, 54, 60, 66, 72, 78, 84, 90, 96, 102, 108, 114, 120, or 126.
   E77. The method of any one of E1-E76, wherein said second antibody comprises:
(1) a CDR-H1, a CDR-H2, a CDR-H3, a CDR-L1, a CDR-L2, and a CDR-L3 comprising SEQ ID NOs. 1-6, respectively;
(2) a CDR-H1, a CDR-H2, a CDR-H3, a CDR-L1, a CDR-L2, and a CDR-L3 comprising SEQ ID NOs. 6-12, respectively;
(3) a CDR-H1, a CDR-H2, a CDR-H3, a CDR-L1, a CDR-L2, and a CDR-L3 comprising SEQ ID NOs. 13-18, respectively;
(4) a CDR-H1, a CDR-H2, a CDR-H3, a CDR-L1, a CDR-L2, and a CDR-L3 comprising SEQ ID NOs. 19-24, respectively;
(5) a CDR-H1, a CDR-H2, a CDR-H3, a CDR-L1, a CDR-L2, and a CDR-L3 comprising SEQ ID NOs. 25-30, respectively;
(6) a CDR-H1, a CDR-H2, a CDR-H3, a CDR-L1, a CDR-L2, and a CDR-L3 comprising SEQ ID NOs. 31-36, respectively;
(7) a CDR-H1, a CDR-H2, a CDR-H3, a CDR-L1, a CDR-L2, and a CDR-L3 comprising SEQ ID NOs. 37-42, respectively;
(8) a CDR-H1, a CDR-H2, a CDR-H3, a CDR-L1, a CDR-L2, and a CDR-L3 comprising SEQ ID NOs. 43-48, respectively;
(9) a CDR-H1, a CDR-H2, a CDR-H3, a CDR-L1, a CDR-L2, and a CDR-L3 comprising SEQ ID NOs. 49-54, respectively;
(10) a CDR-H1, a CDR-H2, a CDR-H3, a CDR-L1, a CDR-L2, and a CDR-L3 comprising SEQ ID NOs. 55-60, respectively;
(11) a CDR-H1, a CDR-H2, a CDR-H3, a CDR-L1, a CDR-L2, and a CDR-L3 comprising SEQ ID NOs. 61-66, respectively;
(12) a CDR-H1, a CDR-H2, a CDR-H3, a CDR-L1, a CDR-L2, and a CDR-L3 comprising SEQ ID NOs. 67-72, respectively;
(13) a CDR-H1, a CDR-H2, a CDR-H3, a CDR-L1, a CDR-L2, and a CDR-L3 comprising SEQ ID NOs. 73-78, respectively;
(14) a CDR-H1, a CDR-H2, a CDR-H3, a CDR-L1, a CDR-L2, and a CDR-L3 comprising SEQ ID NOs. 79-84, respectively;
(15) a CDR-H1, a CDR-H2, a CDR-H3, a CDR-L1, a CDR-L2, and a CDR-L3 comprising SEQ ID NOs. 85-90, respectively;
(16) a CDR-H1, a CDR-H2, a CDR-H3, a CDR-L1, a CDR-L2, and a CDR-L3 comprising SEQ ID NOs. 91-96, respectively;
(17) a CDR-H1, a CDR-H2, a CDR-H3, a CDR-L1, a CDR-L2, and a CDR-L3 comprising SEQ ID NOs. 97-102, respectively;
(18) a CDR-H1, a CDR-H2, a CDR-H3, a CDR-L1, a CDR-L2, and a CDR-L3 comprising SEQ ID NOs. 103-108, respectively;
(19) a CDR-H1, a CDR-H2, a CDR-H3, a CDR-L1, a CDR-L2, and a CDR-L3 comprising SEQ ID NOs. 109-114, respectively;
(20) a CDR-H1, a CDR-H2, a CDR-H3, a CDR-L1, a CDR-L2, and a CDR-L3 comprising SEQ ID NOs. 115-120, respectively; or
(21) a CDR-H1, a CDR-H2, a CDR-H3, a CDR-L1, a CDR-L2, and a CDR-L3 comprising SEQ ID NOs. 121-126, respectively.
   E78. The method of E77, wherein said second antibody comprises a CDR-H1, a CDR-H2, a CDR-H3, a CDR-L1, a CDR-L2, and a CDR-L3 comprising SEQ ID NOs. 1-6, respectively.
   E79. The method of E77, wherein said second antibody comprises a CDR-H1, a CDR-H2, a CDR-H3, a CDR-L1, a CDR-L2, and a CDR-L3 comprising SEQ ID NOs. 6-12, respectively.
   E80. The method of E77, wherein said second antibody comprises a CDR-H1, a CDR-H2, a CDR-H3, a CDR-L1, a CDR-L2, and a CDR-L3 comprising SEQ ID NOs. 13-18, respectively.
   E81. The method of E77, wherein said second antibody comprises a CDR-H1, a CDR-H2, a CDR-H3, a CDR-L1, a CDR-L2, and a CDR-L3 comprising SEQ ID NOs. 19-24, respectively.
   E82. The method of E77, wherein said second antibody comprises a CDR-H1, a CDR-H2, a CDR-H3, a CDR-L1, a CDR-L2, and a CDR-L3 comprising SEQ ID NOs. 25-30, respectively.
   E83. The method of E77, wherein said second antibody comprises a CDR-H1, a CDR-H2, a CDR-H3, a CDR-L1, a CDR-L2, and a CDR-L3 comprising SEQ ID NOs. 31-36, respectively.
   E84. The method of E77, wherein said second antibody comprises a CDR-H1, a CDR-H2, a CDR-H3, a CDR-L1, a CDR-L2, and a CDR-L3 comprising SEQ ID NOs. 37-42, respectively.
   E85. The method of E77, wherein said second antibody comprises a CDR-H1, a CDR-H2, a CDR-H3, a CDR-L1, a CDR-L2, and a CDR-L3 comprising SEQ ID NOs. 43-48, respectively.
   E86. The method of E77, wherein said second antibody comprises a CDR-H1, a CDR-H2, a CDR-H3, a CDR-L1, a CDR-L2, and a CDR-L3 comprising SEQ ID NOs. 49-54, respectively.
   E87. The method of E77, wherein said second antibody comprises a CDR-H1, a CDR-H2, a CDR-H3, a CDR-L1, a CDR-L2, and a CDR-L3 comprising SEQ ID NOs. 55-60, respectively.
   E88. The method of E77, wherein said second antibody comprises a CDR-H1, a CDR-H2, a CDR-H3, a CDR-L1, a CDR-L2, and a CDR-L3 comprising SEQ ID NOs. 61-66, respectively.
   E89. The method of E77, wherein said second antibody comprises a CDR-H1, a CDR-H2, a CDR-H3, a CDR-L1, a CDR-L2, and a CDR-L3 comprising SEQ ID NOs. 67-72, respectively.
   E90. The method of E77, wherein said second antibody comprises a CDR-H1, a CDR-H2, a CDR-H3, a CDR-L1, a CDR-L2, and a CDR-L3 comprising SEQ ID NOs. 73-78, respectively.
   E91. The method of E77, wherein said second antibody comprises a CDR-H1, a CDR-H2, a CDR-H3, a CDR-L1, a CDR-L2, and a CDR-L3 comprising SEQ ID NOs. 79-84, respectively.
   E92. The method of E77, wherein said second antibody comprises a CDR-H1, a CDR-H2, a CDR-H3, a CDR-L1, a CDR-L2, and a CDR-L3 comprising SEQ ID NOs. 85-90, respectively.
   E93. The method of E77, wherein said second antibody comprises a CDR-H1, a CDR-H2, a CDR-H3, a CDR-L1, a CDR-L2, and a CDR-L3 comprising SEQ ID NOs. 91-96, respectively.
   E94. The method of E77, wherein said second antibody comprises a CDR-H1, a CDR-H2, a CDR-H3, a CDR-L1, a CDR-L2, and a CDR-L3 comprising SEQ ID NOs. 97-102, respectively.
   E95. The method of E77, wherein said second antibody comprises a CDR-H1, a CDR-H2, a CDR-H3, a CDR-L1, a CDR-L2, and a CDR-L3 comprising SEQ ID NOs. 103-108, respectively.
   E96. The method of E77, wherein said second antibody comprises a CDR-H1, a CDR-H2, a CDR-H3, a CDR-L1, a CDR-L2, and a CDR-L3 comprising SEQ ID NOs. 109-114, respectively.
   E97. The method of E77, wherein said second antibody comprises a CDR-H1, a CDR-H2, a CDR-H3, a CDR-L1, a CDR-L2, and a CDR-L3 comprising SEQ ID NOs. 115-120, respectively.
   E98. The method of E77, wherein said second antibody comprises a CDR-H1, a CDR-H2, a CDR-H3, a CDR-L1, a CDR-L2, and a CDR-L3 comprising SEQ ID NOs. 121-126, respectively.
   E99. The method of any one of E1-E77, wherein said second antibody comprises a heavy chain variable region (VH) that is at least 90%, at least 91%, at least 92%, at least 93%, at least 94%, at least 95%, at least 96%, at least 97%, at least 98%, at least 99%, or 100% identical to SEQ ID NO: 127, 129, 131, 133, 135, 137, 139, 141, 143, 145, 147, 149, 151, 153, 155, 157, 159, 161, 163, 165, or 167.
   E100. The method of any one of E1-E78, wherein said second antibody comprises a light chain variable region (VL) that is at least 90%, at least 91%, at least 92%, at least 93%, at least 94%, at least 95%, at least 96%, at least 97%, at least 98%, at least 99%, or 100% identical to SEQ ID NO: 128, 130, 132, 134, 136, 138, 140, 142, 144, 146, 148, 150, 152, 154, 156, 158, 160, 162, 164, 166, or 168.
   E101. The method of any one of E1-E100, wherein said second antibody comprises:
(1) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 127, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 128;
(2) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 129, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 130;
(3) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 131, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 132;
(4) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 133, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 134;
(5) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 135, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 136;
(6) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 137, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 138;
(7) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 139, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 140;
(8) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 141, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 142;
(9) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 143, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 144;
(10) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 145, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 146;
(11) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 147, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 148;
(12) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 149, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 150;
(13) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 151, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 152;
(14) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 153, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 154;
(15) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 155, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 156;
(16) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 157, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 158;
(17) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 159, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 160;
(18) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 161, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 162;
(19) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 163, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 164;
(20) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 165, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 166; or
(21) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 167, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 168.
   E102. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 127, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 128.
   E103. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 129, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 130.
   E104. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 131, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 132.
   E105. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 133, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 134.
   E106. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 135, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 136.
   E107. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 137, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 138.
   E108. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 139, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 140.
   E109. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 141, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 142.
   E110. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 143, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 144.
   E111. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 145, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 146.
   E112. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 147, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 148.
   E113. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 149, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 150.
   E114. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 151, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 152.
   E115. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 153, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 154.
   E116. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 155, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 156.
   E117. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 157, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 158.
   E118. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 159, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 160.
   E119. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 161, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 162.
   E120. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 163, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 164.
   E121. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 165, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 166.
   E122. The method of E101, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 167, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 168.
   E123. The method of any one of E1-E122, wherein said second antibody comprises a human CH1 domain, such as a human IgG1 CH1, a human IgG2 CH1, a human IgG3 CH1, ora human IgG4 CH1.
   E124. The method of E123, wherein said human IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:182.
   E125. The method of E123, wherein:
said human IgG2 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:194;
said human IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:190; or
said human IgG4 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:186.
   E126. The method of any one of E1-E125, wherein said second antibody comprises a human Fc domain, such as a human IgG1 Fc, a human IgG2 Fc, a human IgG3 Fc, or a human IgG4 Fc.
   E127. The method of E126, wherein said human IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:184.
   E128. The method of E126, wherein
said human IgG2 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:196;
said human IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:192; or
said human IgG4 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:188.
   E129. The method of any one of E1-E122, wherein said second antibody comprises a murine CH1 domain, such as a murine IgG1 CH1, a murine IgG2a CH1, a murine IgG2b CH1, or a murine IgG3 CH1.
   E130. The method of E129, wherein said murine IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:172.
   E131. The method of E129, wherein said murine IgG2a CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:175.
   E132. The method of E129, wherein said murine IgG2b CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:176.
   E133. The method of E129, wherein said murine IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:177.
   E134. The method of any one of E1-E122 and E129-E133, wherein said second antibody comprises a murine Fc domain, such as a murine IgG1 Fc, a murine IgG2a Fc, a murine IgG2b Fc, or a murine IgG3 Fc.
   E135. The method of E134, wherein said murine IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:174.
   E136. The method of E134, wherein said murine IgG2a Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:175.
   E137. The method of E134, wherein said murine IgG2b Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:176.
   E138. The method of E134, wherein said murine IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:177.
   E139. The method of any one of E1-E138, wherein said second antibody comprises a human CL domain, such as a human kappa CL, or a human lambda CL.
   E140. The method of E139, wherein said human kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:178 or 179.
   E141. The method of E139, wherein said human lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:180 or 181.
   E142. The method of any one of E1-E138, wherein said second antibody comprises a murine CL domain, such as a murine kappa CL, or a murine lambda CL.
   E143. The method of E142, wherein said murine kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:171.
   E144. The method of E142, wherein said murine lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:169 or 170.
   E145. The method of any one of E1-E144, wherein said second antibody binds to RANKL with a KD value of 10⁻⁴ M or less, 10⁻⁵ M or less, 10⁻⁶ M or less, 10⁻⁷ M or less, 10⁻⁸ M or less, or 10⁻⁹ M or less.
   E146. The method of any one of E1-E145, wherein said sample is pre-treated with said second antibody, when mixed with said first antibody or antibody conjugate and said third antibody.
   E147. The method of any one of E1-E145, wherein said sample is pre-treated with said OPG, when mixed with said first antibody or antibody conjugate and said third antibody.
   E148. The method of any one of E1-E145, wherein said sample is mixed with said first antibody or antibody conjugate, said second antibody, and said third antibody simultaneously.
   E149. The method of any one of E1-E145, wherein said sample is mixed with said first antibody or antibody conjugate, said OPG, and said third antibody complex simultaneously.
   E150. The method of any one of E1-E149, wherein said RANKL is human RANKL.
   E151. The method of any one of E1-E150, wherein both said first antibody and second antibody bind to a human RANKL epitope comprising SEQ ID NO:214 (DLATE).
   E152. The method of any one of E1-E150, wherein both said first antibody and second antibody bind to a human RANKL epitope comprising one or more residues from SEQ ID NO:215.
   E153. A diagnostic kit for detecting an anti-drug antibody (ADA) in a sample, wherein said ADA binds to denosumab, comprising:
(a) a first antibody or antibody conjugate that binds to RANKL, comprising: the heavy chain (HC) CDR-H1, CDR-H2, and CDR-H3 of SEQ ID NO:203, and the light chain (LC) CDR-L1, CDR-L2, and CDR-L3 of SEQ ID NO:208; wherein said first antibody or antibody conjugate can be attached to a solid substrate;
(b) (i) a second antibody that binds to RANKL, wherein said second antibody comprises an amino acid sequence that differs from the first antibody by at least one amino acid in one of the six CDR sequences; or (ii) an osteoprotegerin (OPG) protein that binds to RANKL;
(c) a third antibody that binds to said ADA and can be linked to a detectable marker; and
(d) instructions for use.
   E154. The diagnostic kit of E153, wherein said first antibody conjugate comprises an antibody molecule covalently linked to an immobilizing moiety.
   E155. The diagnostic kit of E153 or E154, wherein said first antibody conjugate comprises an antibody molecule noncovalently linked to an immobilizing moiety.
   E156. The diagnostic kit of E154 or E155, wherein said solid substrate comprises a binding partner, and said immobilizing moiety can be attached to a solid substrate by binding to said binding partner.
   E157. The diagnostic kit of E156, wherein said immobilizing moiety is biotin and said binding partner is streptavidin or avidin; or said immobilizing moiety is avidin or streptavidin and said binding partner is biotin.
   E158. The diagnostic kit of E156, wherein said immobilizing moiety and said binding partner are one of the following binding pairs: an antigen and an antibody that binds to said antigen; a lectin and a polysaccharide, a steroid and a steroid binding protein, a hormone and a hormone receptor, an enzyme and a substrate of the enzyme, an IgG and Protein A, an IgG and Protein G; or a ligand and a receptor for the ligand.
   E159. The diagnostic kit of E153, wherein said first antibody is immobilized to a solid substrate that comprises Protein A or Protein G.
   E160. The diagnostic kit of any one E153-E159, wherein said first antibody or antibody conjugate comprise a CDR-H1 comprising SEQ ID NO:200, a CDR-H2 comprising SEQ ID NO:201, and a CDR-H3 comprising SEQ ID NO:202; and a CDR-L1 comprising SEQ ID NO:205, a CDR-L2 comprising SEQ ID NO:206, and a CDR-L3 comprising SEQ ID NO:207.
   E161. The diagnostic kit of any one E153-E160, wherein said first antibody or antibody conjugate comprises a heavy chain variable region (VH) that is at least 90%, at least 91%, at least 92%, at least 93%, at least 94%, at least 95%, at least 96%, at least 97%, at least 98%, at least 99%, or 100% identical to SEQ ID NO:203.
   E162. The diagnostic kit of any one E153-E161, wherein said first antibody or antibody conjugate comprises a light chain variable region (VL) that is at least 90%, at least 91%, at least 92%, at least 93%, at least 94%, at least 95%, at least 96%, at least 97%, at least 98%, at least 99%, or 100% identical to SEQ ID NO:208.
   E163. The diagnostic kit of any one E153-E162, wherein said first antibody or antibody conjugate comprises a human CH1 domain, such as a human IgG1 CH1, a human IgG2 CH1, a human IgG3 CH1, or a human IgG4 CH1.
   E164. The diagnostic kit of E163, wherein said human IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:182.
   E165. The diagnostic kit of E163, wherein:
said human IgG2 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:194;
said human IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:190; or
said human IgG4 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:186.
   E166. The diagnostic kit of any one of E153-E165, wherein said first antibody or antibody conjugate comprises a human Fc domain, such as a human IgG1 Fc, a human IgG2 Fc, a human IgG3 Fc, or a human IgG4 Fc.
   E167. The diagnostic kit of E166, wherein said human IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:184.
   E168. The diagnostic kit of E166, wherein:
said human IgG2 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:196;
said human IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:192; or
said human IgG4 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:188.
   E169. The diagnostic kit of any one of E153-E162, wherein said first antibody or antibody conjugate comprises a murine CH1 domain, such as a murine IgG1 CH1, a murine IgG2a CH1, a murine IgG2b CH1, or a murine IgG3 CH1.
   E170. The diagnostic kit of E169, wherein said murine IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:172.
   E171. The diagnostic kit of E169, wherein said murine IgG2a CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:175.
   E172. The diagnostic kit of E169, wherein said murine IgG2b CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:176.
   E173. The diagnostic kit of E169, wherein said murine IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:177.
   E174. The diagnostic kit of any one of E153-E162 and E169-E173, wherein said first antibody or antibody conjugate comprises a murine Fc domain, such as a murine IgG1 Fc, a murine IgG2a Fc, a murine IgG2b Fc, or a murine IgG3 Fc.
   E175. The diagnostic kit of E174, wherein said murine IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 74.
   E176. The diagnostic kit of E174, wherein said murine IgG2a Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:175.
   E177. The diagnostic kit of E174, wherein said murine IgG2b Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:176.
   E178. The diagnostic kit of E174, wherein said murine IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:177.
   E179. The diagnostic kit of any one of E153-E178, wherein said first antibody or antibody conjugate comprises a human CL domain, such as a human kappa CL, or a human lambda CL.
   E180. The diagnostic kit of E179, wherein said human kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:178 or 179.
   E181. The diagnostic kit of E179, wherein said human lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:180 or 181.
   E182. The diagnostic kit of any one of E153-E178, wherein said first antibody or antibody conjugate comprises a murine CL domain, such as a murine kappa CL, or a murine lambda CL.
   E183. The diagnostic kit of E182, wherein said murine kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:171.
   E184. The diagnostic kit of E182, wherein said murine lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:169 or 170.
   E185. The diagnostic kit of any one of E153-E184, wherein said third antibody is covalently linked to a detectable marker.
   E186. The diagnostic kit of any one of E153-E184, wherein said third antibody is noncovalently linked to a detectable marker.
   E187. The diagnostic kit of any one of E153-E186, wherein said detectable marker is an isotope, an enzyme, a fluorescent moiety, a luminescent moiety, a chromatic moiety, a metal, or an electric charge.
   E188. The diagnostic kit of any one of E153-E187, wherein said third antibody comprises six CDR sequences that are identical to that of the first antibody.
   E189. The diagnostic kit of any one E153-E188, wherein said third antibody comprise:
(i) the heavy chain (HC) CDR-H1, CDR-H2, and CDR-H3 of SEQ ID NO:203, and the light chain (LC) CDR-L1, CDR-L2, and CDR-L3 of SEQ ID NO:208; or
(ii) a CDR-H1 comprising SEQ ID NO:200, a CDR-H2 comprising SEQ ID NO:201, and a CDR-H3 comprising SEQ ID NO:202; and a CDR-L1 comprising SEQ ID NO:205, a CDR-L2 comprising SEQ ID NO:206, and a CDR-L3 comprising SEQ ID NO:207.
   E190. The diagnostic kit of any one of E153-E189, wherein said third antibody comprises a heavy chain variable region (VH) that is at least 90%, at least 91%, at least 92%, at least 93%, at least 94%, at least 95%, at least 96%, at least 97%, at least 98%, at least 99%, or 100% identical to SEQ ID NO:203.
   E191. The diagnostic kit of any one of E153-E190, wherein said third antibody comprises a light chain variable region (VL) that is at least 90%, at least 91%, at least 92%, at least 93%, at least 94%, at least 95%, at least 96%, at least 97%, at least 98%, at least 99%, or 100% identical to SEQ ID NO:208.
   E192. The diagnostic kit of any one of E153-E191, wherein said third antibody comprises a human CH1 domain, such as a human IgG1 CH1, a human IgG2 CH1, a human IgG3 CH1, ora human IgG4 CH1.
   E193. The diagnostic kit of E192, wherein said human IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:182.
   E194. The diagnostic kit of E192, wherein:
said human IgG2 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:194;
said human IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:190; or
said human IgG4 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:186.
   E195. The diagnostic kit of any one of E192-E194, wherein said third antibody comprises a human Fc domain, such as a human IgG1 Fc, a human IgG2 Fc, a human IgG3 Fc, or a human IgG4 Fc.
   E196. The diagnostic kit of E195, wherein said human IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:184.
   E197. The diagnostic kit of E195, wherein
said human IgG2 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:196;
said human IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:192; or
said human IgG4 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:188.
   E198. The diagnostic kit of any one of E153-E191, wherein said third antibody comprises a murine CH1 domain, such as a murine IgG1 CH1, a murine IgG2a CH1, a murine IgG2b CH1, or a murine IgG3 CH1.
   E199. The diagnostic kit of E198, wherein said murine IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:172.
   E200. The diagnostic kit of E198, wherein said murine IgG2a CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:175.
   E201. The diagnostic kit of E198, wherein said murine IgG2b CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:176.
   E202. The diagnostic kit of E198, wherein said murine IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:177.
   E203. The diagnostic kit of any one of E153-E191 and E198-E202, wherein said third antibody comprises a murine Fc domain, such as a murine IgG1 Fc, a murine IgG2a Fc, a murine IgG2b Fc, or a murine IgG3 Fc.
   E204. The diagnostic kit of E203, wherein said murine IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:174.
   E205. The diagnostic kit of E203, wherein said murine IgG2a Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:175.
   E206. The diagnostic kit of E203, wherein said murine IgG2b Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:176.
   E207. The diagnostic kit of E203, wherein said murine IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:177.
   E208. The diagnostic kit of any one of E153-E207, wherein said third antibody comprises a human CL domain, such as a human kappa CL, or a human lambda CL.
   E209. The diagnostic kit of E208, wherein said human kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:178 or 179.
   E210. The diagnostic kit of E208, wherein said human lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:180 or 181.
   E211. The diagnostic kit of any one of E153-E207, wherein said third antibody comprises a murine CL domain, such as a murine kappa CL, or a murine lambda CL.
   E212. The diagnostic kit of E211, wherein said murine kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:171.
   E213. The diagnostic kit of E211, wherein said murine lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:169 or 170.
   E214. The diagnostic kit of any one of E153-E213, wherein said third antibody comprises a VH domain comprising the same sequence as the VH of the first antibody, and a VL domain comprising the same sequence as the VL domain of the first antibody.
   E215. The diagnostic kit of any one of E153-E214, wherein said third antibody comprises a heavy chain (HC) comprising the same sequence as the HC of the first antibody, and a light chain (LC) comprising the same sequence as the LC of the first antibody.
   E216. The diagnostic kit of any one of E153-E215, wherein said detectable marker comprises a secondary antibody that binds to a constant domain of said third antibody.
   E217. The diagnostic kit of any one of E153-E216, wherein said detectable marker comprises a secondary antibody that binds to the Fc domain of said third antibody.
   E218. The diagnostic kit of any one of E153-E215, wherein said third antibody is covalently linked to a fluorescent moiety, a luminescent moiety, or a chromatic moiety.
   E219. The diagnostic kit of any one of E153-E218, wherein said second antibody binds to the same epitope as denosumab.
   E220. The diagnostic kit of any one of E153-E219, wherein said second antibody competes with denosumab for RANKL binding.
   E221. The diagnostic kit of any one of E153-E220, wherein said second antibody comprises an amino acid sequence that differs from the first antibody by at least one amino acid in each of the six CDR sequences.
   E222. The diagnostic kit of any one of E153-E221, wherein said second antibody comprises an amino acid sequence that differs from the first antibody by at least two, at least three, at least four, at least five, at least six amino acids in CDR sequences.
   E223. The diagnostic kit of any one of E153-E222, wherein said ADA does not bind to said second antibody.
   E224. The diagnostic kit of any one of E153-E223, wherein said second antibody comprises: (i) the heavy chain CDR-H1, CDR-H2, and CDR-H3 of SEQ ID NO: 127, 129, 131, 133, 135, 137, 139, 141, 143, 145, 147, 149, 151, 153, 155, 157, 159, 161, 163, 165, or 167; and (ii) the light chain CDR-L1, CDR-L2, and CDR-L3 of SEQ ID NO: 128, 130, 132, 134, 136, 138, 140, 142, 144, 146, 148, 150, 152, 154, 156, 158, 160, 162, 164, 166, or 168.
   E225. The diagnostic kit of any one of E153-E224, wherein said second antibody comprises:
(i) a CDR-H1 comprising a sequence that is at least 90%, at least 91%, at least 92%, at least 93%, at least 94%, at least 95%, at least 96%, at least 97%, at least 98%, at least 99%, or 100% identical to SEQ ID NO: 1, 7, 13, 19, 25, 31, 37, 43, 49, 55, 61, 67, 73, 79, 85, 91, 97, 103, 109, 115, or 121;
(ii) a CDR-H2 comprising a sequence that is at least 90%, at least 91%, at least 92%, at least 93%, at least 94%, at least 95%, at least 96%, at least 97%, at least 98%, at least 99%, or 100% identical to SEQ ID NO: 2, 8, 14, 20, 26, 32, 38, 44, 50, 56, 62, 68, 74, 80, 86, 92, 98, 104, 110, 116, or 122;
(iii) a CDR-H3 comprising a sequence that is at least 90%, at least 91%, at least 92%, at least 93%, at least 94%, at least 95%, at least 96%, at least 97%, at least 98%, at least 99%, or 100% identical to SEQ ID NO:3, 9, 15, 21, 27, 33, 39, 45, 51, 57, 63, 69, 75, 81, 87, 93, 99, 105, 111, 117, or 123;
(iv) a CDR-L1 comprising a sequence that is at least 90%, at least 91%, at least 92%, at least 93%, at least 94%, at least 95%, at least 96%, at least 97%, at least 98%, at least 99%, or 100% identical to SEQ ID NO:4, 10, 16, 22, 28, 34, 40, 46, 52, 58, 64, 70, 76, 82, 88, 94, 100, 106, 112, 118, or 124;
(v) a CDR-L2 comprising a sequence that is at least 90%, at least 91%, at least 92%, at least 93%, at least 94%, at least 95%, at least 96%, at least 97%, at least 98%, at least 99%, or 100% identical to SEQ ID NO:5, 11, 17, 23, 29, 35, 41, 47, 53, 59, 65, 71, 77, 83, 89, 95, 101, 107, 113, 119, or 125; and
(vi) a CDR-L3 comprising a sequence that is at least 90%, at least 91%, at least 92%, at least 93%, at least 94%, at least 95%, at least 96%, at least 97%, at least 98%, at least 99%, or 100% identical to SEQ ID NO:6, 12, 18, 24, 30, 36, 42, 48, 54, 60, 66, 72, 78, 84, 90, 96, 102, 108, 114, 120, or 126.
   E226. The diagnostic kit of any one of E153-E225, wherein said second antibody comprises:
(1) a CDR-H1, a CDR-H2, a CDR-H3, a CDR-L1, a CDR-L2, and a CDR-L3 comprising SEQ ID NOs. 1-6, respectively;
(2) a CDR-H1, a CDR-H2, a CDR-H3, a CDR-L1, a CDR-L2, and a CDR-L3 comprising SEQ ID NOs. 6-12, respectively;
(3) a CDR-H1, a CDR-H2, a CDR-H3, a CDR-L1, a CDR-L2, and a CDR-L3 comprising SEQ ID NOs. 13-18, respectively;
(4) a CDR-H1, a CDR-H2, a CDR-H3, a CDR-L1, a CDR-L2, and a CDR-L3 comprising SEQ ID NOs. 19-24, respectively;
(5) a CDR-H1, a CDR-H2, a CDR-H3, a CDR-L1, a CDR-L2, and a CDR-L3 comprising SEQ ID NOs. 25-30, respectively;
(6) a CDR-H1, a CDR-H2, a CDR-H3, a CDR-L1, a CDR-L2, and a CDR-L3 comprising SEQ ID NOs. 31-36, respectively;
(7) a CDR-H1, a CDR-H2, a CDR-H3, a CDR-L1, a CDR-L2, and a CDR-L3 comprising SEQ ID NOs. 37-42, respectively;
(8) a CDR-H1, a CDR-H2, a CDR-H3, a CDR-L1, a CDR-L2, and a CDR-L3 comprising SEQ ID NOs. 43-48, respectively;
(9) a CDR-H1, a CDR-H2, a CDR-H3, a CDR-L1, a CDR-L2, and a CDR-L3 comprising SEQ ID NOs. 49-54, respectively;
(10) a CDR-H1, a CDR-H2, a CDR-H3, a CDR-L1, a CDR-L2, and a CDR-L3 comprising SEQ ID NOs. 55-60, respectively;
(11) a CDR-H1, a CDR-H2, a CDR-H3, a CDR-L1, a CDR-L2, and a CDR-L3 comprising SEQ ID NOs. 61-66, respectively;
(12) a CDR-H1, a CDR-H2, a CDR-H3, a CDR-L1, a CDR-L2, and a CDR-L3 comprising SEQ ID NOs. 67-72, respectively;
(13) a CDR-H1, a CDR-H2, a CDR-H3, a CDR-L1, a CDR-L2, and a CDR-L3 comprising SEQ ID NOs. 73-78, respectively;
(14) a CDR-H1, a CDR-H2, a CDR-H3, a CDR-L1, a CDR-L2, and a CDR-L3 comprising SEQ ID NOs. 79-84, respectively;
(15) a CDR-H1, a CDR-H2, a CDR-H3, a CDR-L1, a CDR-L2, and a CDR-L3 comprising SEQ ID NOs. 85-90, respectively;
(16) a CDR-H1, a CDR-H2, a CDR-H3, a CDR-L1, a CDR-L2, and a CDR-L3 comprising SEQ ID NOs. 91-96, respectively;
(17) a CDR-H1, a CDR-H2, a CDR-H3, a CDR-L1, a CDR-L2, and a CDR-L3 comprising SEQ ID NOs. 97-102, respectively;
(18) a CDR-H1, a CDR-H2, a CDR-H3, a CDR-L1, a CDR-L2, and a CDR-L3 comprising SEQ ID NOs. 103-108, respectively;
(19) a CDR-H1, a CDR-H2, a CDR-H3, a CDR-L1, a CDR-L2, and a CDR-L3 comprising SEQ ID NOs. 109-114, respectively;
(20) a CDR-H1, a CDR-H2, a CDR-H3, a CDR-L1, a CDR-L2, and a CDR-L3 comprising SEQ ID NOs. 115-120, respectively; or
(21) a CDR-H1, a CDR-H2, a CDR-H3, a CDR-L1, a CDR-L2, and a CDR-L3 comprising SEQ ID NOs. 121-126, respectively.
   E227. The diagnostic kit of E226, wherein said second antibody comprises a CDR-H1, a CDR-H2, a CDR-H3, a CDR-L1, a CDR-L2, and a CDR-L3 comprising SEQ ID NOs. 1-6, respectively.
   E228. The diagnostic kit of E226, wherein said second antibody comprises a CDR-H1, a CDR-H2, a CDR-H3, a CDR-L1, a CDR-L2, and a CDR-L3 comprising SEQ ID NOs. 6-12, respectively.
   E229. The diagnostic kit of E226, wherein said second antibody comprises a CDR-H1, a CDR-H2, a CDR-H3, a CDR-L1, a CDR-L2, and a CDR-L3 comprising SEQ ID NOs. 13-18, respectively.
   E230. The diagnostic kit of E226, wherein said second antibody comprises a CDR-H1, a CDR-H2, a CDR-H3, a CDR-L1, a CDR-L2, and a CDR-L3 comprising SEQ ID NOs. 19-24, respectively.
   E231. The diagnostic kit of E226, wherein said second antibody comprises a CDR-H1, a CDR-H2, a CDR-H3, a CDR-L1, a CDR-L2, and a CDR-L3 comprising SEQ ID NOs. 25-30, respectively.
   E232. The diagnostic kit of E226, wherein said second antibody comprises a CDR-H1, a CDR-H2, a CDR-H3, a CDR-L1, a CDR-L2, and a CDR-L3 comprising SEQ ID NOs. 31-36, respectively.
   E233. The diagnostic kit of E226, wherein said second antibody comprises a CDR-H1, a CDR-H2, a CDR-H3, a CDR-L1, a CDR-L2, and a CDR-L3 comprising SEQ ID NOs. 37-42, respectively.
   E234. The diagnostic kit of E226, wherein said second antibody comprises a CDR-H1, a CDR-H2, a CDR-H3, a CDR-L1, a CDR-L2, and a CDR-L3 comprising SEQ ID NOs. 43-48, respectively.
   E235. The diagnostic kit of E226, wherein said second antibody comprises a CDR-H1, a CDR-H2, a CDR-H3, a CDR-L1, a CDR-L2, and a CDR-L3 comprising SEQ ID NOs. 49-54, respectively.
   E236. The diagnostic kit of E226, wherein said second antibody comprises a CDR-H1, a CDR-H2, a CDR-H3, a CDR-L1, a CDR-L2, and a CDR-L3 comprising SEQ ID NOs. 55-60, respectively.
   E237. The diagnostic kit of E226, wherein said second antibody comprises a CDR-H1, a CDR-H2, a CDR-H3, a CDR-L1, a CDR-L2, and a CDR-L3 comprising SEQ ID NOs. 61-66, respectively.
   E238. The diagnostic kit of E226, wherein said second antibody comprises a CDR-H1, a CDR-H2, a CDR-H3, a CDR-L1, a CDR-L2, and a CDR-L3 comprising SEQ ID NOs. 67-72, respectively.
   E239. The diagnostic kit of E226, wherein said second antibody comprises a CDR-H1, a CDR-H2, a CDR-H3, a CDR-L1, a CDR-L2, and a CDR-L3 comprising SEQ ID NOs. 73-78, respectively.
   E240. The diagnostic kit of E226, wherein said second antibody comprises a CDR-H1, a CDR-H2, a CDR-H3, a CDR-L1, a CDR-L2, and a CDR-L3 comprising SEQ ID NOs. 79-84, respectively.
   E241. The diagnostic kit of E226, wherein said second antibody comprises a CDR-H1, a CDR-H2, a CDR-H3, a CDR-L1, a CDR-L2, and a CDR-L3 comprising SEQ ID NOs. 85-90, respectively.
   E242. The diagnostic kit of E226, wherein said second antibody comprises a CDR-H1, a CDR-H2, a CDR-H3, a CDR-L1, a CDR-L2, and a CDR-L3 comprising SEQ ID NOs. 91-96, respectively.
   E243. The diagnostic kit of E226, wherein said second antibody comprises a CDR-H1, a CDR-H2, a CDR-H3, a CDR-L1, a CDR-L2, and a CDR-L3 comprising SEQ ID NOs. 97-102, respectively.
   E244. The diagnostic kit of E226, wherein said second antibody comprises a CDR-H1, a CDR-H2, a CDR-H3, a CDR-L1, a CDR-L2, and a CDR-L3 comprising SEQ ID NOs. 103-108, respectively.
   E245. The diagnostic kit of E226, wherein said second antibody comprises a CDR-H1, a CDR-H2, a CDR-H3, a CDR-L1, a CDR-L2, and a CDR-L3 comprising SEQ ID NOs. 109-114, respectively.
   E246. The diagnostic kit of E226, wherein said second antibody comprises a CDR-H1, a CDR-H2, a CDR-H3, a CDR-L1, a CDR-L2, and a CDR-L3 comprising SEQ ID NOs. 115-120, respectively.
   E247. The diagnostic kit of E226, wherein said second antibody comprises a CDR-H1, a CDR-H2, a CDR-H3, a CDR-L1, a CDR-L2, and a CDR-L3 comprising SEQ ID NOs. 121-126, respectively.
   E248. The diagnostic kit of any one of E153-E247, wherein said second antibody comprises a heavy chain variable region (VH) that is at least 90%, at least 91%, at least 92%, at least 93%, at least 94%, at least 95%, at least 96%, at least 97%, at least 98%, at least 99%, or 100% identical to SEQ ID NO: 127, 129, 131, 133, 135, 137, 139, 141, 143, 145, 147, 149, 151, 153, 155, 157, 159, 161, 163, 165, or 167.
   E249. The diagnostic kit of any one of E153-E248, wherein said second antibody comprises a light chain variable region (VL) that is at least 90%, at least 91%, at least 92%, at least 93%, at least 94%, at least 95%, at least 96%, at least 97%, at least 98%, at least 99%, or 100% identical to SEQ ID NO: 128, 130, 132, 134, 136, 138, 140, 142, 144, 146, 148, 150, 152, 154, 156, 158, 160, 162, 164, 166, or 168.
   E250. The diagnostic kit of any one of E153-E249, wherein said second antibody comprises:
(1) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 127, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 128;
(2) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 129, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 130;
(3) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 131, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 132;
(4) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 133, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 134;
(5) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 135, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 136;
(6) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 137, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 138;
(7) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 139, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 140;
(8) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 141, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 142;
(9) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 143, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 144;
(10) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 145, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 146;
(11) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 147, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 148;
(12) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 149, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 150;
(13) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 151, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 152;
(14) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 153, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 154;
(15) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 155, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 156;
(16) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 157, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 158;
(17) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 159, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 160;
(18) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 161, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 162;
(19) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 163, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 164;
(20) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 165, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 166; or
(21) a VH comprising a sequence that is at least 90%, at least 91%, at least 92%, at least 93%, at least 94%, at least 95%, at least 96%, at least 97%, at least 98%, at least 99%, or 100% identical to SEQ ID NO: 167, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 168.
   E251. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 127, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 128.
   E252. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 129, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 130.
   E253. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 131, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 132.
   E254. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 133, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 134.
   E255. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 135, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 136.
   E256. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 137, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 138.
   E257. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 139, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 140.
   E258. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 141, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 142.
   E259. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 143, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 144.
   E260. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 145, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 146.
   E261. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 147, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 148.
   E262. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 149, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 150.
   E263. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 151, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 152.
   E264. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 153, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 154.
   E265. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 155, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 156.
   E266. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 157, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 158.
   E267. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 159, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 160.
   E268. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 161, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 162.
   E269. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 163, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 164.
   E270. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 165, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 166.
   E271. The diagnostic kit of E250, wherein said second antibody comprises a VH comprising a sequence that is at least 90%, at least 91%, at least 92%, at least 93%, at least 94%, at least 95%, at least 96%, at least 97%, at least 98%, at least 99%, or 100% identical to SEQ ID NO: 167, and a VL comprising a sequence that is at least 90%, at least 91%, at least 92%, at least 93%, at least 94%, at least 95%, at least 96%, at least 97%, at least 98%, at least 99%, or 100% identical to SEQ ID NO: 168.
   E272. The diagnostic kit of any one of E153-E271, wherein said second antibody comprises a human CH1 domain, such as a human IgG1 CH1, a human IgG2 CH1, a human IgG3 CH1, or a human IgG4 CH1.
   E273. The diagnostic kit of E272, wherein said human IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:182.
   E274. The diagnostic kit of E272, wherein:
said human IgG2 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:194;
said human IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:190; or
said human IgG4 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:186.
   E275. The diagnostic kit of any one of E153-E274, wherein said second antibody comprises a human Fc domain, such as a human IgG1 Fc, a human IgG2 Fc, a human IgG3 Fc, or a human IgG4 Fc.
   E276. The diagnostic kit of E275, wherein said human IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:184.
   E277. The diagnostic kit of E275, wherein
said human IgG2 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:196;
said human IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:192; or
said human IgG4 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:188.
   E278. The diagnostic kit of any one of E153-E271, wherein said second antibody comprises a murine CH1 domain, such as a murine IgG1 CH1, a murine IgG2a CH1, a murine IgG2b CH1, or a murine IgG3 CH1.
   E279. The diagnostic kit of E278, wherein said murine IgG1 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:172.
   E280. The diagnostic kit of E278, wherein said murine IgG2a CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:175.
   E281. The diagnostic kit of E278, wherein said murine IgG2b CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:176.
   E282. The diagnostic kit of E278, wherein said murine IgG3 CH1 comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the CH1 domain of SEQ ID NO:177.
   E283. The diagnostic kit of any one of E153-E271 and E278-E282, wherein said second antibody comprises a murine Fc domain, such as a murine IgG1 Fc, a murine IgG2a Fc, a murine IgG2b Fc, or a murine IgG3 Fc.
   E284. The diagnostic kit of E283, wherein said murine IgG1 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:174.
   E285. The diagnostic kit of E283, wherein said murine IgG2a Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:175.
   E286. The diagnostic kit of E283, wherein said murine IgG2b Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:176.
   E287. The diagnostic kit of E283, wherein said murine IgG3 Fc comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the Fc domain of SEQ ID NO:177.
   E288. The diagnostic kit of any one of E153-E287, wherein said second antibody comprises a human CL domain, such as a human kappa CL, or a human lambda CL.
   E289. The diagnostic kit of E288, wherein said human kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:178 or 179.
   E290. The diagnostic kit of E288, wherein said human lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:180 or 181.
   E291. The diagnostic kit of any one of E153-E287, wherein said second antibody comprises a murine CL domain, such as a murine kappa CL, or a murine lambda CL.
   E292. The diagnostic kit of E291, wherein said murine kappa CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:171.
   E293. The diagnostic kit of E291, wherein said murine lambda CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:169 or 170.
   E294. The diagnostic kit of any one of E153-E293, wherein said second antibody binds to RANKL with a KD value of 10⁻⁴ M or less, 10⁻⁵ M or less, 10⁻⁶ M or less, 10⁻⁷ M or less, 10⁻⁸ M or less, or 10⁻⁹ M or less.
   E295. The diagnostic kit of any one of E153-E294, wherein said RANKL is human RANKL.
   E296. The diagnostic kit of any one of E153-E295, wherein both said first antibody and second antibody binds to a human RANKL epitope comprising SEQ ID NO:214 (DLATE).
   E297. The diagnostic kit of any one of E153-E295, wherein both said first antibody and second antibody binds to a human RANKL epitope comprising one or more residues from SEQ ID NO:215.
   E298. A diagnosis kit for use in a method of any one of E1-E152.

### BRIEF DESCRIPTION OF THE DRAWINGS

FIG. 1A illustrates an exemplary ADA assay. FIG. 1B illustrates that soluble RANKL interferes with the ADA assay.

FIGS. 2A and 2B illustrate that a second anti-RANKL antibody, or OPG, can reduce signal interference by sequestering soluble RANKL.

### DETAILED DESCRIPTION

### 1. Anti-Drug Antibody (ADA) Assays Overview

In one aspect, the invention provides a method of detecting an anti-drug antibody (ADA) against denosumab in a sample. In general, the sample is obtained from a patient who has been treated with denosumab.

Denosumab (trade names Prolia® and Xgeva®) is a human monoclonal antibody that target RANKL. It comprises a heavy chain comprising SEQ ID NO:198, and a light chain comprising SEQ ID NO:199. Prolia is indicated for the treatment of postmenopausal women with osteoporosis at high risk for fracture, the treatment to increase bone mass in men with osteoporosis at high risk for fracture, the treatment of glucocorticoid-induced osteoporosis in men and women at high risk of fracture, the treatment to increase bone mass in men at high risk for fracture receiving androgen deprivation therapy for nonmetastatic prostate cancer, and the treatment to increase bone mass in women at high risk for fracture receiving adjuvant aromatase inhibitor therapy for breast cancer. Xgeva is a prescription medicine used to prevent fracture, spinal cord compression, or the need for radiation or surgery to bone in patients with multiple myeloma and in patients with bone metastases from solid tumors.

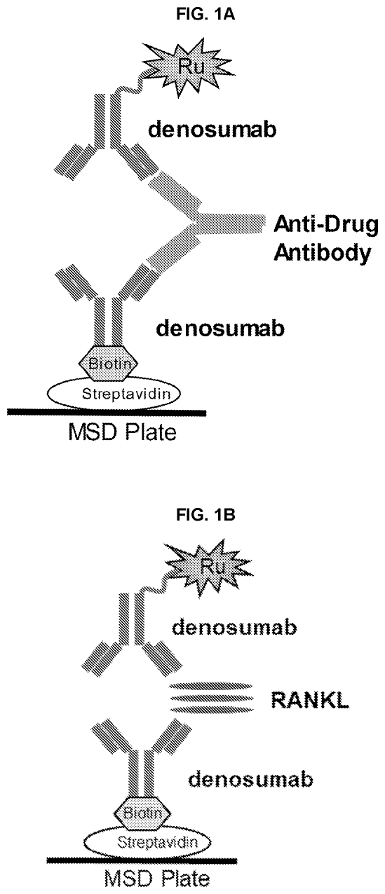

As taught and exemplified in the Examples, in one study, an unusually high incidence of immunoreactivity in the anti-drug antibody (ADA) assay against denosumab was observed. Based on past experience with denosumab clinical studies, this was believed to be false positive and likely due to nuclear factor-KB ligand (RANKL) interfering with the ADA assay (FIGS. 1A and 1B).

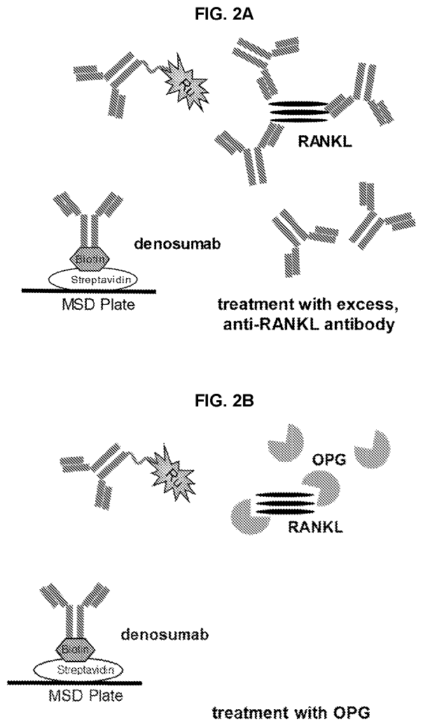

To address the false positive signals caused by soluble RANKL and to develop a reliable ADA assay, an exemplary anti-RANKL monoclonal antibody that competes with denosumab for binding to RANKL was incorporated into an improved ADA assay. Because the complementarity determining regions (CDRs) of an antibody are the result of random mutations from germline, which then undergo a selection process, the CDRs are the most likely domain to be recognized by the immune system as foreign. Consequently, most anti-drug antibodies to monoclonal antibody therapeutics are CDR-directed. Based on these data, it is believed that to reduce the interference of RANKL ligand in an ADA assay, two strategies maybe used: (i) using OPG (which is a decoy receptor for RANKL) to sequester soluble RANKL; or (ii) using an antibody that binds to RANKL to sequester RANKL; this antibody should have different CDR sequences than denosumab, such that the antibody itself does not bind to ADA (FIGS. 2A and 2B).

### 2. Immunoassays for Detecting ADAs

The most commonly used ADA assays are based on solid-phase immunoassays. Standard solid-phase immunoassays with monoclonal antibodies involve the formation of a complex between an antibody adsorbed on a solid substrate (“capture” molecule), molecule of interest (in this case, ADA against denosumab), and an antibody linked to a detectable marker (“tracer” molecule). Thus, a sandwich is formed: solid substrate-capture molecule-molecule of interest-tracer molecule. In the sandwich, typically the amount of the detectable marker is proportional to the concentration of the ADA in sample. One sandwich assay is the double antigen bridging immunoassay whereby capture and tracer antibodies bind to different epitopes of the antigen. Hoesel, W., et al., in J. Immunol. Methods 294 (2004) 101-110, report an anti-EPO double antigen bridging assay whereby a mixture of immobilized rhEPO coupled to amino groups and to carbohydrate groups was used. Immunoassays such as the double antigen bridging ELISA are common assay types in the investigation of an immunogenic answer of a patient to an antibody drug. Mire-Sluis, A. R., et al., in J. Immunol. Methods 289 (2004) 1-16, summarize the recommendations for the design and optimization of immunoassays using detection of host antibodies against biotechnology products.

### Capture Molecules

A “capture” molecule is typically an antibody that is covalently or non-covalently linked to an immobilizing moiety, and the solid substrate comprises a binding partner that binds to said immobilizing moiety. Through the interaction between the immobilizing moiety and its binding partner, the capture molecule can be immobilized to a solid substate. The capture molecule is also referred to as the “first antibody or antibody conjugate” herein.

Exemplary binding pairs that can be used as an immobilizing moiety and its binding partner include for example (first component-second component): streptavidin-biotin, avidin-biotin, antibody-antigen (see, for example, Hermanson, G. T., et al., Bioconjugate Techniques, Academic Press, 1996), lectin-polysaccharide, steroid-steroid binding protein, hormone-hormone receptor, enzyme-substrate, IgG-Protein A, IgG-Protein G, and ligand-receptor. In an exemplary embodiment, the capture molecule is an antibody conjugated to biotin and immobilization is performed via Avidin or Streptavidin that attached to the surface of a solid substrate.

In certain embodiments, conjugation of the antibody to the immobilizing moiety is by covalent conjugation via N-terminal and/or s-amino groups (lysine), ε-amino groups of different lysines, carboxy-, sulfhydryl-, hydroxyl- and/or phenolic functional groups of the amino acid backbone of the antibody and/or sugar alcohol groups of the carbohydrate structure of the antibody.

Since it is believed that ADAs are often caused by CDR sequences, to detect ADAs against denosumab, the capture molecule should comprise an antibody that has the same CDR sequences of denosumab. The amino acid sequences of the heavy chain (HC), light chain (LC), heavy chain variable domain (VH), light chain variable domain (VL), heavy chain complementarity determining regions (CDR-H1, CDR-H2, CDR-H3), and light chain complementarity determining regions (CDR-L1, CDR-L2, CDR-L3) of denosumab is shown in Table F of the Sequence Table.

As used herein, “Complementarity Determining Regions” (CDRs) can be identified according to the definitions of the Kabat, Chothia, the accumulation of both Kabat and Chothia, AbM, contact, North, and/or conformational definitions or any method of CDR determination well known in the art. See, e.g., Kabat et al., 1991, Sequences of Proteins of Immunological Interest, 5th ed. (hypervariable regions); Chothia et al., 1989, Nature 342:877-883 (structural loop structures). The identity of the amino acid residues in a particular antibody that make up a CDR can be determined using methods well known in the art. AbM definition of CDRs is a compromise between Kabat and Chothia and uses Oxford Molecular's AbM antibody modeling software (Accelrys®). The “contact” definition of CDRs is based on observed antigen contacts, set forth in MacCallum et al., 1996, J. Mol. Biol., 262:732-745. The “conformational” definition of CDRs is based on residues that make enthalpic contributions to antigen binding (see, e.g., Makabe et al., 2008, J. Biol. Chem., 283:1 156-1 166). North has identified canonical CDR conformations using a different preferred set of CDR definitions (North et al., 2011, J. Mol. Biol. 406: 228-256). In another approach, referred to herein as the “conformational definition” of CDRs, the positions of the CDRs may be identified as the residues that make enthalpic contributions to antigen binding (Makabe et al., 2008, J Biol. Chem. 283:1 156-1 166). Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs (or other residue of the antibody) may be defined in accordance with any of Kabat, Chothia, North, extended, AbM, contact, and/or conformational definitions.

For example, the following Table shows several commonly used definitions of CDRs:

| Loop | Kabat | AbM | Chothia¹ | Contact² | IMGT |
|---|---|---|---|---|---|
| L1 | L24-L34 | L24-L34 | L26-L32 | L30-L36 | L27-L32 |
| L2 | L50-L56 | L50-L56 | L50-L52 | L46-L55 | L50-L51 |
| L3 | L89-L97 | L89-L97 | L91-L96 | L89-L96 | L89-L97 |
| H1 | H31-H35B | H26-H35B | H26-H32 . . . 34 | H30-H35B | H26-H35B |
| | (Kabat Numbering)³ | | | | |
| H1 | H31-H35 | H26-H35 | H26-H32 | H30-H35 | H26-H33 |
| | (Chothia Numbering) | | | | |
| H2 | H50-H65 | H50-H58 | H52-H56 | H47-H58 | H51-H56 |
| H3 | H95-H102 | H95-H102 | H96-H101 | H93-H101 | H93-H102 |

¹some of these definitions (particularly for Chothia loops) vary depending on the individual publication examined. Some papers describe Chothia CDRs as: CDR-L1:L24-34; CDR-L2:L50-56; CDR-L3:L89-97; CDR-H1:H26-32; CDR-H2:H52-56; CDR-H3:H95-102.
²Any of the numbering schemes can be used for these CDR definitions, except the contact definition uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)

The CDR sequences provided in the Sequence Table are based on the Kabat definition. However, other definitions for CDRs may also be used. Accordingly, in some embodiments, the first antibody comprises the CDR-H1, CDR-H2, and CDR-H3 sequences of SEQ ID NO:203, and the CDR-L1, CDR-L2, and CDR-L3 sequences of SEQ ID NO:208. In some embodiments, the first antibody comprises a CDR-H1 comprising SEQ ID NO:200, a CDR-H2 comprising SEQ ID NO:201, and a CDR-H3 comprising SEQ ID NO:202; and a CDR-L1 comprising SEQ ID NO:205, a CDR-L2 comprising SEQ ID NO:206, and a CDR-L3 comprising SEQ ID NO:207.

Outside the CDR regions, the sequence of the first antibody may differ from that of denosumab; nonetheless, for convenience, denosumab VH and/or VL sequences may be used. Accordingly, in some embodiments, the first antibody or antibody conjugate comprises a heavy chain variable region (VH) that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:203, and/or a light chain variable region (VL) that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:208.

The constant region of the first antibody (or the capture antibody) may be human or murine. Examples of human and murine constant region sequences are provided in the Sequence Table E. For purpose of conducting assays with human samples, in some cases certain murine constant region sequences may be preferred. This could sometimes reduce the cross-reaction or false positives. Endogenous human antibodies within the sample would not interfere with appropriate detection of the ADA molecules. Accordingly, in some embodiments, the first antibody or antibody conjugate comprises a human CH1 domain (such as a human IgG1 CH1, a human IgG2 CH1, a human IgG3 CH1, or a human IgG4 CH1); a human Fc domain (such as a human IgG1 Fc, a human IgG2 Fc, a human IgG3 Fc, or a human IgG4 Fc); a murine IgG1 CH1 (such as a murine IgG2a CH1, a murine IgG2b CH1, or a murine IgG3 CH1); a murine IgG1 Fc (such as a murine IgG2a Fc, a murine IgG2b Fc, or a murine IgG3 Fc), a human CL domain (such as a human kappa CL, or a human lambda CL), or a murine CL domain (such as a murine kappa CL, or a murine lambda CL).

In an exemplary embodiment, the first antibody or antibody conjugate comprises denosumab conjugated to biotin.

The heavy and light chain sequences of denosumab are provided as SEQ ID NOs. 198 and 199, respectively. It should be noted that often, during recombinant production process, terminal lysine and glycine of the heavy chain may be clipped. C-terminal lysine clipping is a common phenomenon occurring during the bioproduction of monoclonal antibodies. Often, the lysine residue is removed via carboxypeptidase D (CpD), which results in generation of a mixture of antibody isoforms bearing zero or one C-terminal lysine residues on each heavy chain. Further, following C-terminal lysine cleavage, peptidylglycine α-amidating monooxygenase (PAM) catalyzes the hydroxylation of glycine and removal of the glyoxylate from the glycine residue, leaving an amidated C-terminal proline. Therefore, during recombinant production of a monoclonal antibody, the product is often a mixture of C-terminal processing variants, with heavy chain C-terminus ends at (amidated) proline, glycine, or lysine. Therefore, in some embodiments, the terminal lysine of SEQ ID NO:198 may be absent; in some embodiments, the terminal lysine of SEQ ID NO:198 may be present; in some embodiments, the terminal glycine-lysine of SEQ ID NO:198 may be absent; in some embodiments, the terminal glycine-lysine of SEQ ID NO:198 may be present.

“Solid substrate” refers to a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; cuvettes, tubes, or other spectrometer sample containers. In an ADA assay, solid substrate refers to inert solid surfaces that in general contains a “binding partner” (e.g., avidin or streptavidin) on its surface, which is intended to interact with the “capture” antibody; in this case the first antibody or antibody conjugate comprising an immobilizing moiety (e.g., biotin) that is recognized by the “binding partner.” A solid phase may be a stationary component, such as a tube, strip, cuvette, or microtiter plate, or may be a non-stationary component, such as beads and microparticles. Microparticles can also be used as a solid phase for homogeneous assay formats. A variety of microparticles that allow either non-covalent or covalent attachment of proteins and other substances may be used. Such particles include polymer particles such as polystyrene and poly(methylmethacrylate); gold particles such as gold nanoparticles and gold colloids; and ceramic particles such as silica, glass, and metal oxide particles. See for example Martin, C. R., et al., Analytical Chemistry-News & Features 70 (1998) 322A-327A.

In general, the first antibody or antibody conjugate is immobilized to the solid substrate by passive adsorption and therefore attached to the solid phase at a site that does not interfere with its binding to ADA. Passive adsorption is, e.g., described by Butler, J. E., Solid Phases in Immunoassay, In: Immunoassays, Diamandis, E. P. and Christopoulos, T. K. (eds.) Academic Press San Diego (1996), pp. 205-225.

### Tracer Molecules

In a typically ADA assay, the “tracer” molecule (also referred herein as the “third antibody”) needs to be able to bind to the ADA molecule to form a sandwich (e.g., see FIG. 1A). The most convenient way is to have the same CDR sequences as the first antibody, since ADA is typically caused by CDR sequences. The tracer molecule is also linked to a detectable marker to facilitate the detection of ADA.

Accordingly, in certain embodiments, the third antibody comprise the same six CDRs as denosumab. As described in detail above, CDRs can be identified according to the definitions of the Kabat, Chothia, the accumulation of both Kabat and Chothia, AbM, contact, North, and/or conformational definitions or any method of CDR determination well known in the art. The CDR sequences provided in the Sequence Table are based on the Kabat definition. However, other definitions for CDRs may also be used. Accordingly, in some embodiments, the third antibody comprises an antibody comprising the CDR-H1, CDR-H2, and CDR-H3 sequences of SEQ ID NO:203, and the CDR-L1, CDR-L2, and CDR-L3 sequences of SEQ ID NO:208. In some embodiments, the third antibody comprises an antibody comprising a CDR-H1 comprising SEQ ID NO:200, a CDR-H2 comprising SEQ ID NO:201, and a CDR-H3 comprising SEQ ID NO:202; and a CDR-L1 comprising SEQ ID NO:205, a CDR-L2 comprising SEQ ID NO:206, and a CDR-L3 comprising SEQ ID NO:207.

Outside the CDR regions, the sequence of the third antibody may differ from that of denosumab; nonetheless, for convenience, denosumab VH and/or VL sequences may be used. Accordingly, in some embodiments, the third antibody comprises a heavy chain variable region (VH) that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:203, and/or a light chain variable region (VL) that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:208.

The constant region of the third antibody (or the tracer antibody) may be human or murine. Examples of human and murine constant region sequences are provided in the Sequence Table E. For purpose of conducting assays with human samples, in some cases certain murine constant region sequences may be preferred. This could sometimes reduce the cross-reaction or false positives. Endogenous human antibodies within the sample would not interfere with appropriate detection of the ADA molecules. Accordingly, in some embodiments, the third antibody comprises a human CH1 domain (such as a human IgG1 CH1, a human IgG2 CH1, a human IgG3 CH1, or a human IgG4 CH1); a human Fc domain (such as a human IgG1 Fc, a human IgG2 Fc, a human IgG3 Fc, or a human IgG4 Fc); a murine IgG1 CH1 (such as a murine IgG2a CH1, a murine IgG2b CH1, ora murine IgG3 CH1); a murine IgG1 Fc (such as a murine IgG2a Fc, a murine IgG2b Fc, or a murine IgG3 Fc), a human CL domain (such as a human kappa CL, or a human lambda CL), or a murine CL domain (such as a murine kappa CL, or a murine lambda CL).

In some embodiments, the third antibody comprises a VH domain comprising the same sequence as the VH of the first antibody, and a VL domain comprising the same sequence as the VL domain of the first antibody. In some embodiments, the third antibody comprises a heavy chain (HC) comprising the same sequence as the HC of the first antibody, and a light chain (LC) comprising the same sequence as the LC of the first antibody.

Typically, the “tracer” molecule (or “the third antibody” herein) is covalently or noncovalently linked to a detectable marker. The detectable marker can be an isotope, an enzyme, a fluorescent moiety, a luminescent moiety, a chromatic moiety, a metal, or an electric charge. For example, the detectable marker can be an electrochemiluminescent label, like a ruthenium bispyridyl complex. Chromogens (fluorescent or luminescent groups and dyes), enzymes, NMR-active groups or metal particles, haptens, such as, e.g., digoxigenin, are also examples of detectable markers. The detectable marker can also be a photoactivatable crosslinking group, e.g. an azido or an azirine group. Metal chelates which can be detected by electrochemoluminescence are also preferred signal-emitting groups, with particular preference being given to ruthenium chelates, e.g. a ruthenium (bispyridyl)3 2+ chelate. Suitable ruthenium labeling groups are described, for example, in EP 0 580 979, WO 90/05301, WO 90/11511, and WO 92/14138. Further, nearly any antibody can be labeled with biotin, HRP enzyme or one of several fluorophores if needed.

Sometimes, indirect detection method is used. In this case, the “tracer” molecule is considered as a “primary antibody”, which binds to ADA, and the primary antibody is not labeled for direct detection. Instead, a “secondary antibody” that has been labeled with a detectable tag is applied in a second step to probe for the primary antibody-ADA complex. Thus, the ADA is detected indirectly. Another form of indirect detection involves using a primary or secondary antibody that is labeled with an affinity tag such as biotin. Then a secondary (or tertiary) probe, such as streptavidin that is labeled with the detectable enzyme or fluorophore tag, can be used to probe for the biotin tag to yield a detectable signal. Several variants of these probing and detection strategies exist. In general, the strategy depends on a specific probe (e.g., a primary antibody) whose presence is linked directly or indirectly to some sort of detectable marker (e.g., an enzyme whose activity can produce a colored product upon reaction with its substrate).

Most primary antibodies can be produced or manipulated to have mouse, rabbit or one of several other non-human constant domains. Many of these are antibodies of the IgG class, in particular, IgG1 subclass. Therefore, it is relatively easy and economical for manufacturers to produce and supply ready-to-use, labeled secondary antibodies for most applications and detection systems. The choice of secondary antibody depends upon the species of animal in which the primary antibody was raised (the host species). For example, if the primary antibody has a mouse constant domain, then the secondary antibody should be an anti-mouse antibody obtained from a host other than the mouse. Off the shelf secondary antibodies that bind to a constant domain or the Fc domain of primary antibodies are in general readily available.

Biotin-based systems are often used as detectable markers. With biotin-binding proteins as probes, the highly specific affinity interaction between biotin and avidin or streptavidin protein is the basis for many kinds of detection and affinity-purification methods. Biotin is very small (244 Daltons), so its covalent attachment to antibodies or other probes rarely interferes with their functions. Yet its presence as a label on a probe allows efficient and specific secondary detection with either avidin or streptavidin. Both kinds of biotin-binding proteins are available in purified forms labeled with enzymatic or fluorescent tags that enable detection in many kinds of assays systems.

Enzymatic labels are commonly used as secondary antibody (or streptavidin) tags for detection in blotting and immunoassays. Enzymes provide detectable signal via their activity; reaction with a specific substrate chemical yields a colored, light-emitting, or fluorescent product. While reporter enzymes like beta-galactosidase and luciferase have been successfully used to make probes, alkaline phosphatase (AP) and horseradish peroxidase (HRP) are the two enzymes used most extensively as labels for protein detection. An array of chromogenic, fluorogenic and chemiluminescent substrates is available for use with either enzyme.

Alkaline phosphatase (AP), usually isolated from calf intestine, is a large (140 kDa) protein that catalyzes the hydrolysis of phosphate groups from a substrate molecule resulting in a colored or fluorescent product or the release of light as a byproduct of the reaction. AP has optimal enzymatic activity at a basic pH (pH 8-10) and can be inhibited by cyanides, arsenate, inorganic phosphate and divalent cation chelators, such as EDTA. AP offers certain advantage as a detectable marker. Because its reaction rate remains linear, detection sensitivity can be improved by simply allowing a reaction to proceed for a longer time period.

Horseradish peroxidase (HRP) is a 40 kDa protein that catalyzes the oxidation of substrates by hydrogen peroxide, resulting in a colored or fluorescent product or the release of light as a byproduct of the reaction. HRP functions optimally at a near-neutral pH and can be inhibited by cyanides, sulfides and azides. Antibody-HRP conjugates have shown to be superior to antibody-AP conjugates with respect to the specific activities of both the enzyme and antibody. In addition, its high turnover rate, good stability, low cost and wide availability of substrates makes HRP the enzyme of choice for many applications. Because of the small size of the HRP enzyme, further increases in sensitivity may be achieved by using poly-HRP conjugated secondary antibodies.

Two common fluorophores for labeling probes are fluorescein (fluorescein isothiocyanate, FITC) and rhodamine (tetramethyl rhodamine isothiocyanate, TRITC).

Other detectable markers include fluorescent proteins such as the various forms of green fluorescent protein (GFP) and the phycobiliproteins (allophycocyanin, phycocyanin, phycoerythrin and phycoerythrocyanin).

### Reducing Signal Interference

As disclosed herein, in an exemplary ADA assay to detect denosumab ADA, a large number of samples showed false positive signals, and it was believed to be caused by RANKL interfering with the ADA assay (FIGS. 1A and 1B).

The sequence of human RANKL, including membraned-bound and soluble forms of RANKL are provided in the sequence table F. The soluble form of human RANKL comprises residues 69-317 of the membrane-bound form (as numbered in WO 98/46757), shown as SEQ ID NOs:212 and 213. Proteolytic cleavage between residues 139 and 140 of the membrane-bound RANKL also yields a soluble form as shown in SEQ ID NO: 214.

To address the false positive signals caused by soluble RANKL, it is important to include in the ADA assay a molecule that can sequester soluble RANKL. Two strategies are provided herein, one uses a second anti-RANKL antibody, and the other uses decoy receptor OPG.

To effectively sequester RANKL without interfering with the detection of ADA, the second antibody should have the following characteristics: (1) it should have different CDR sequences than denosumab (since ADAs are often CDR-directed, having different CDRs generally ensures that there is no cross-reaction between the second antibody and ADA); and (2) it should bind to RANKL. Preferably, the second antibody binds to the same epitope of RANKL as denosumab; or the second antibody competes (or at least partially competes) with denosumab for RANKL binding. When the second antibody and denosumab cannot bind to RANKL simultaneously, the signal interference caused by RANKL can be reduced most effectively. Nonetheless, competing (or at least partially competing) with denosumab for RANKL binding is not an absolute requirement. For example, in Examples 3-6, the exemplary anti-RANKL mAb was found to not compete with denosumab for RANKL for binding. Epitope binning showing that this anti-RANKL mAb binds to a different epitope bin and does not block denosumab or OPG for RANKL binding. A skilled artisan will be able to readily assess the binding behaviors of the second antibody and design the ADA assays accordingly based on common knowledge. Example 2B also provides an example on how to assess and select suitable second antibodies.

In addition, although it is preferred that the second antibody competes (or at least partially competes) with denosumab for RANKL binding, it is not necessary that the second antibody binds to RANKL with similar affinity (KD value) as denosumab. Even a poor competitor with low binding affinity for RANKL would be adequate to reduce/eliminate the interference. In that case, the competing antibody can be spiked into the serum sample in large quantities and only needs to sequester a small amount of RANKL.

KD is the equilibrium dissociation constant, a ratio of k_{off}/kₒₙ, between the antigen binding protein and its target or antigen. KD and KA are inversely related. The KD value relates to the concentration of the antibody (the amount of antibody needed for a particular experiment) and so the lower the KD value (lower concentration needed) the higher the affinity of the antibody. In exemplary aspects, the KD value of the second antibody to RANKL is about 10⁻¹ M or less, about 10⁻² M or less, about 10⁻³ M or less, about 10⁻⁴ M or less, about 10⁻⁵ M or less, about 10⁻⁶ M or less, about 10⁻⁷ M or less, about 10⁻¹ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10⁻⁵ M to about 10⁻¹⁵ M, from about 10⁻⁶ M to about 10⁻¹⁵ M, from about 10⁻⁷ M to about 10⁻¹⁵ M, from about 10⁻¹ M to about 10⁻¹⁵ M, from about 10⁻⁹ M to about 10⁻¹⁵ M, from about 10⁻¹⁰ M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻⁵ M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, or from about 10⁻¹⁰ M to about 10⁻¹³ M.

In exemplary aspects, the KD of the second anti-RANKL antibody is micromolar, nanomolar, picomolar or femtomolar. In exemplary aspects, the KD is within a range of about 10⁻⁴ to 10⁻⁶ M, or 10⁻⁷ to 10⁻⁹ M, or 10⁻¹⁰ to 10⁻¹² M, or 10⁻¹³ to 10⁻¹⁵ M. In exemplary aspects, the second antibody binds to the human RANKL with a KD value of: about 1 uM or less, about 900 nM or less, about 800 nM or less, about 700 nM or less, about 600 nM or less, about 500 nM or less, about 400 nM or less, about 300 nM or less, about 200 nM or less, about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 5 nM or less, about 2 nM or less, about 1 nM or less, about 900 pM or less, about 800 pM or less, about 700 pM or less, about 600 pM or less, about 500 pM or less, about 400 pM or less, about 300 pM or less, about 250 pM or less, about 200 pM or less, about 150 pM or less, about 100 pM or less, about 50 pM or less, about 40 pM or less, about 30 pM or less, about 25 pM or less, about 20 pM or less, about 15 pM or less, about 10 pM or less, about 5 pM or less, or about 1 pM or less.

KD values can be determined using methods well established in the art. One exemplary method for measuring KD is surface plasmon resonance (SPR), a method well-known in the art (e.g., Nguyen et al. Sensors (Basel). 2015 May 5; 15(5):10481-510). KD value may be measured by SPR using a biosensor system such as a BIACORE® system. BIAcore kinetic analysis comprises analyzing the binding and dissociation of an antigen from chips with immobilized molecules (e.g. molecules comprising epitope binding domains), on their surface. Another well-known method in the art for determining the KD of a protein is by using Bio-Layer Interferometry (e.g., Shah et al. J Vis Exp. 2014; (84): 51383). KD value may be measured by Bio-Layer Interferometry using OCTET® technology (Octet QKe system, ForteBio). Alternatively or in addition, a KinExA® (Kinetic Exclusion Assay) assay, available from Sapidyne Instruments (Boise, Id.) can also be used. Any method known in the art for assessing the binding affinity between two binding partners is encompassed herein.

In some aspects, the KD value is measured by surface plasmon resonance (SPR). Antigen (RANKL) may be immobilized, e.g., on a solid surface. The antigen may be immobilized to a chip, for example by covalent coupling (such as amine coupling). The chip may be a CM5 sensor chip. As the analyte binds to the ligand the accumulation of protein on the sensor surface causes an increase in refractive index. This refractive index change is measured in real time (sampling in a kinetic analysis experiment is taken every 0.1 s), and the result plotted as response units (RU) versus time (termed a sensorgram). A response (background response) will also be generated if there is a difference in the refractive indices of the running and sample buffers. This background response must be subtracted from the sensorgram to obtain the actual binding response. The background response is recorded by injecting the analyte through a control or reference flow cell, which has no ligand or an irrelevant ligand immobilized to the sensor surface. The real-time measurement of association and dissociation of a binding interaction allows for the calculation of association and dissociation rate constants and the corresponding affinity constants. One RU represents the binding of 1 pg of protein per square mm. More than 50 pg per square mm of analyte binding is generally needed in practice to generate good reproducible responses.

Dissociation of the antibody from the antigen may be monitored for about 3600 seconds. The SPR analysis may be conducted, and the data collected at between about 15° C. and about 37° C. The SPR analysis may be conducted, and the data collected at between about 25° C. and 37° C. The SPR analysis may be conducted, and the data collected at about 37° C. The SPR analysis may be conducted, and the data collected at 37° C. The K_{D} value may be measured by SPR using a BIAcore T200 instrument. The SPR rates and affinities may be determined by fitting resulting sensorgram data to a 1:1 model in BIAcore T200 Evaluation software version 1.0. The collection rate may be about 1 Hz.

Another method for determining the KD of an antibody is by using Bio-Layer Interferometry (BLI), typically using OCTET® technology (Octet QKe system, ForteBio). In some embodiments, biosensor analysis is used. Typically, one interactant is immobilized on the surface of the biosensor (“ligand,” such as an antibody) and the other remains in solution (“analyte”, such as an antigen). The assay begins with an initial baseline or equilibration step using assay buffer. Next, a ligand (such as an antibody) is immobilized on the surface of the biosensor (loading), either by direct immobilization or capture-based method. After ligand immobilization, biosensors are dipped into buffer solution for a baseline step to assess assay drift and determine loading level of ligand. After the baseline step, biosensors are dipped into a solution containing the ligand's binding partner, the analyte (association). In this step, the binding interaction of the analyte to the immobilized ligand is measured. Following analyte association, the biosensor is dipped into buffer solution without analyte, and the bound analyte is allowed to come off the ligand (dissociation). The series of assay steps is then repeated on new or regenerated biosensors for each analyte being tested. Each binding response is measured and reported in real time on a sensorgram trace. The instrument may be Octet QKe system, Octet RED96 system, Octet QK384 system, or RED384 system.

In certain aspects, the second antibody binds to the same RANKL epitope as denosumab. Denosumab is known to bind to an epitope comprising a portion of the amino acid sequence of a DE region of RANKL (DE epitope). The DE region of RANKL spans approximately the D and E beta sheet regions and intervening loop sequence (DE loop). The DE region in human RANKL comprises from about amino acid residue 212 to about amino acid residue 250 (GFYYLYANICFRHHETSGDLATEYLQLMVYVTKTSIKIP, SEQ ID NO: 216). However, the amino acid sequence and endpoints of the DE region of human RANKL are merely exemplary, and it is understood that DE regions may have sequences and endpoints which vary. In particular, it has been shown that within DE loop of human RANKL, the amino acid sequence DLATE (SEQ ID NO:215) is important for denosumab binding. See, e.g., WO2001062932.

Accordingly, in some embodiments, the second anti-RANKL antibody binds to an epitope comprising the sequence of “DLATE” (SEQ ID NO:215). in some embodiments, the second anti-RANKL antibody binds to an epitope that is located within DE loop of human RANKL.

An “epitope” refers to the area or region of an antigen to which an antibody specifically binds, e.g., an area or region comprising residues that interacts with the antibody. Epitopes can be linear or conformational. The term “paratope” is derived from the above definition of “epitope” by reversing the perspective, and refers to the area or region of an antibody molecule which is involved in binding of an antigen, e.g., an area or region comprising residues that interacts with the antigen. A paratope may be linear or conformational (such as discontinuous residues in CDRs). The epitope/paratope for a given antibody/antigen binding pair can be defined and characterized at different levels of detail using a variety of experimental and computational epitope mapping methods. The experimental methods include mutagenesis, X-ray crystallography, Nuclear Magnetic Resonance (NMR) spectroscopy, Hydrogen/deuterium exchange Mass Spectrometry (HX-MS) and various competition binding methods. As each method relies on a unique principle, the description of an epitope is intimately linked to the method by which it has been determined. Thus, the epitope/paratope for a given antibody/antigen pair will be defined differently depending on the mapping method employed.

From the fact that descriptions and definitions of epitopes, dependent on the epitope mapping method used, and obtained at different levels of detail, it follows that comparison of epitopes for different antibodies on the same antigen can similarly be conducted at different levels of detail. For example, epitopes described on the amino acid level, e.g., determined from an X-ray structure, are said to be identical if they contain the same set of amino acid residues. Epitopes characterized by competition binding are said to be overlapping if the binding of the corresponding antibodies are mutually exclusive, i.e., binding of one antibody excludes simultaneous or consecutive binding of the other antibody; and epitopes are said to be separate (unique) if the antigen is able to accommodate binding of both corresponding antibodies simultaneously.

The epitope and paratope for a given antibody/antigen pair may be identified by routine methods. For example, the general location of an epitope may be determined by assessing the ability of an antibody to bind to different fragments RANKL.

In certain aspect, the second antibody competes with denosumab for RANKL binding. The term “compete,” as used herein with regard to an antibody, means that binding of a first antibody, or an antigen-binding portion thereof, to an antigen reduces the subsequent binding of the same antigen by a second antibody or an antigen-binding portion thereof. In general, the binding a first antibody creates steric hindrance, conformational change, or binding to a common epitope (or portion thereof), such that the binding of the second antibody to the same antigen is reduced. Standard competition assays may be used to determine whether two antibodies compete with each other. One suitable assay for antibody competition involves the use of the Biacore technology, which can measure the extent of interactions using surface plasmon resonance (SPR) technology, typically using a biosensor system (such as a BIACORE® system). For example, SPR can be used in an in vitro competitive binding inhibition assay to determine the ability of one antibody to inhibit the binding of a second antibody.

Another assay for measuring antibody competition uses an ELISA-based approach. Furthermore, a high throughput process for “binning” antibodies based upon their competition is described in International Patent Application No. WO2003/48731. Competition is present if one antibody (or fragment) reduces the binding of another antibody (or antigen-binding fragment thereof to RANKL. For example, a sequential binding competition assay may be used, with different antibodies being added sequentially. The first antibody may be added to reach binding that is close to saturation. Then, the second antibody is added. If the binding of second antibody to RANKL is not detected, or is significantly reduced (e.g., at least about 10%, at least about 20%, at least about 30%, at least about 40%, at least about 50%, at least about 60%, at least about 70%, at least about 80%, or at least about 90% reduction) as compared to a parallel assay in the absence of the first antibody (which value can be set as 100%), the two antibodies are considered as competing with each other.

Exemplary anti-RANKL antibodies that can be used in the ADA assays disclosed herein are shown in Sequence Tables. Accordingly, in certain embodiments, the second antibody comprises: (i) the heavy chain CDR-H1, CDR-H2, and CDR3-H3 of SEQ ID NO: 127, 129, 131, 133, 135, 137, 139, 141, 143, 145, 147, 149, 151, 153, 155, 157, 159, 161, 163, 165, or 167; and (ii) the light chain CDR-L1, CDR-L2, and CDR3 of SEQ ID NO: 128, 130, 132, 134, 136, 138, 140, 142, 144, 146, 148, 150, 152, 154, 156, 158, 160, 162, 164, 166, or 168. As described in detail above, CDRs can be identified according to the definitions of the Kabat, Chothia, the accumulation of both Kabat and Chothia, AbM, contact, North, and/or conformational definitions or any method of CDR determination well known in the art. The CDR sequences provided in the Sequence Table are based on the Kabat definition. However, other definitions for CDRs may also be used.

In some embodiments, the second antibody comprises (i) a CDR-H1 comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1, 7, 13, 19, 25, 31, 37, 43, 49, 55, 61, 67, 73, 79, 85, 91, 97, 103, 109, 115, or 121; (ii) a CDR-H2 comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2, 8, 14, 20, 26, 32, 38, 44, 50, 56, 62, 68, 74, 80, 86, 92, 98, 104, 110, 116, or 122; (iii) a CDR-H3 comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:3, 9, 15, 21, 27, 33, 39, 45, 51, 57, 63, 69, 75, 81, 87, 93, 99, 105, 111, 117, or 123; (iv) a CDR-L1 comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:4, 10, 16, 22, 28, 34, 40, 46, 52, 58, 64, 70, 76, 82, 88, 94, 100, 106, 112, 118, or 124; (v) a CDR-L2 comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:5, 11, 17, 23, 29, 35, 41, 47, 53, 59, 65, 71, 77, 83, 89, 95, 101, 107, 113, 119, or 125; and (vi) a CDR-L3 comprising a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:6, 12, 18, 24, 30, 36, 42, 48, 54, 60, 66, 72, 78, 84, 90, 96, 102, 108, 114, 120, or 126.

In some embodiments, the second antibody comprises a heavy chain variable region (VH) that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 127, 129, 131, 133, 135, 137, 139, 141, 143, 145, 147, 149, 151, 153, 155, 157, 159, 161, 163, 165, or 167. In some embodiments, the second antibody comprises a light chain variable region (VL) that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 128, 130, 132, 134, 136, 138, 140, 142, 144, 146, 148, 150, 152, 154, 156, 158, 160, 162, 164, 166, or 168.

The constant region of the second antibody may be human or murine. Examples of human and murine constant region sequences are provided in the Sequence Table. For purpose of conducing assays with human samples, in some cases certain murine constant region sequences may be preferred. This could sometimes reduce the cross-reaction or false positives. Endogenous antibodies within the sample would not interfere with appropriate detection of the ADA molecules. Accordingly, in some embodiments, the third antibody comprises a human CH1 domain (such as a human IgG1 CH1, a human IgG2 CH1, a human IgG3 CH1, or a human IgG4 CH1); a human Fc domain (such as a human IgG1 Fc, a human IgG2 Fc, a human IgG3 Fc, or a human IgG4 Fc); a murine IgG1 CH1 (such as a murine IgG2a CH1, a murine IgG2b CH1, or a murine IgG3 CH1); a murine IgG1 Fc (such as a murine IgG2a Fc, a murine IgG2b Fc, or a murine IgG3 Fc), a human CL domain (such as a human kappa CL, or a human lambda CL), or a murine CL domain (such as a murine kappa CL, or a murine lambda CL).

Alternatively, Osteoprotegerin (OPG) may be used to sequester soluble RANKL. OPG is also known as osteoclastogenesis inhibitory factor (OCIF) or tumor necrosis factor receptor superfamily member 11B (TNFRSF11B), is a cytokine receptor of the tumor necrosis factor (TNF) receptor superfamily encoded by the TNFRSF11B gene. OPG is a decoy receptor for RANKL. The exemplary sequence of human OPG is shown in Sequence Table F.

In certain embodiments, the OPG is human OPG. In certain embodiment, the OPG comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:210 or 211.

The samples may be pre-treated with the second anti-RANKL antibody or OPG disclosed herein to remove soluble RANKL, then, the first antibody or antibody conjugate and the third antibody may be added to complete the ADA assay. Alternatively, the first antibody or antibody conjugate, the second antibody or OPG, and the third antibody may be added simultaneously. This is particularly feasible when the second antibody competes or partially competes with the first antibody for RANKL binding (i.e., when the first antibody and the second antibody cannot simultaneously bind to RANKL). When the first antibody and the second antibody cannot simultaneously bind to RANKL, the presence of second antibody will prevent RANKL from bridging the immobilized first antibody and the detectable marker.

Excess amount of the second antibody or OPG may be added to ensure the complete sequestration of soluble RANKL in the sample. In certain embodiments, excess amount of the second antibody or OPG is added to a sample, such that the molar ratio of first antibody to second antibody or OPG is at least 1:2, at least 1:3, at least 1:4, at least 1:5, at least 1:6, at least 1:7, at least 1:8, at least 1:9, at least 1:10, at least 1:15, at least 1:20, at least 1:30, at least 1:40, at least 1:50, at least 1:60, at least 1:70, at least 1:80, at least 1:90, at least 1:100, at least 1:200, at least 1:300, at least 1:400, at least 1:500, at least 1:600, at least 1:700, at least 1:800, at least 1:900, or at least 1:1000.

### 3. Diagnostic Kits

In certain aspects, this disclosure provides kits that are adapted for determining the presence or absence of an ADA against denosumab in a sample. The kits may comprise instructions and reagents for the ADA assays disclosed herein. For example, the kit may comprise: (a) a first antibody or antibody conjugate that binds to RANKL, comprising: the heavy chain (HC) CDR-H1, CDR-H2, and CDR-H3 of SEQ ID NO:203, and the light chain (LC) CDR-L1, CDR-L2, and CDR-L3 of SEQ ID NO:208; wherein said first antibody or antibody conjugate can be attached to a solid substrate; (b) (i) a second antibody that binds to RANKL, wherein said second antibody comprises an amino acid sequence that differs from the first antibody by at least one amino acid in one of the six CDR sequences; or (ii) an osteoprotegerin (OPG) protein that binds to RANKL; (c) a third antibody that binds to said ADA and can be linked to a detectable marker; and (d) instructions for use.

The kit may also comprise an immobilizing moiety that can be covalently or non-covalently linked to the first antibody. The kit may also comprise a solid substrate, and a binding partner that can bind to the immobilizing moiety. The binding partner may already be fixed to the substrate, or reagents are provided to allow a user to attach the binding partner to the solid substrate.

The kit may also comprise a detectable marker. Reagents that allow a user to covalently or non-covalently link the third antibody to the detectable marker may also be included. Alternatively, the kid may comprise a third antibody that is already attached to a detectable marker covalently or non-covalently.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purpose

### EXAMPLES

### Example 1. Background and Rationale

In one denosumab bioequivalence study (“Study A”), an unusually high incidence of immunoreactivity in the anti-drug antibody assay (ADA) was observed. Based on past experience with denosumab clinical studies, this was unexpected, and an investigation was initiated. False positive results due to target interference was suspected because the positive samples gave signal that was uniform and low magnitude, more in line with treatment-induced shedding of receptor activator of nuclear factor-KB ligand (RANKL) than an anti-denosumab antibody response.

To investigate this possibility, a subset of antibody positive samples from Study A were treated with osteoprotegerin (OPG), a decoy receptor for RANKL, and retested in the antibody assay. The hypothesis was that if the antibody-positive results were due to soluble RANKL, OPG treatment would reduce the assay signal by competing with denosumab for binding. If the antibody-positive results were caused by anti-denosumab antibodies, the OPG treatment would have no impact on the assay signal, as OPG does not bind to ADA molecules. Specifically, as shown in FIGS. 1A-1B, in an exemplary MSD ADA assay, an anti-drug antibody can bridge the biotin-labeled-denosumab and SULFO-TAG (ruthenium tris bipyridine) labeled denosumab. The complexes are immobilized using streptavidin-coated plates and detected through chemiluminescence (FIG. 1A). It is possible that soluble RANKL trimers can also bridge the labeled denosumab molecules (FIG. 1B) and cause false positive signals. If this is the case, pre-treating the sample with OPG can deplete the soluble RANKL from the samples and eliminate the false positive signals. Indeed, it was confirmed that the OPG treatment led to a clear reduction in signal in most antibody-positive samples tested (data not shown), indicating that many of the positive results were due to target (RANKL) interference.

To address the false positive signals caused by soluble RANKL and to develop a reliable ADA assay, an anti-RANKL monoclonal antibody that binds to human RANKL was incorporated into an improved ADA assay. Because the complementarity determining regions (CDRs) of an antibody are the result of random mutations from germline, which then undergo a selection process, the CDRs may be recognized by the immune system as foreign. Consequently, most anti-drug antibodies to monoclonal antibody therapeutics are CDR-directed. For this reason, screening and selection of an anti-RANKL antibody with a different CDR than denosumab was conducted to ensure that the incorporated anti-RANKL antibody did not inadvertently bind ADA, inhibiting the signal.

### Example 2. Materials and Methods for Denosumab ADA Assays

Meso Scale Discovery (MSD) based bridging immunoassay method. Serum samples were diluted 1:20 in 300 mM acetic acid to enable antibody complex dissociation prior to analysis. Diluted samples were then incubated with a conjugate/anti-RANKL/neutralization mixture comprising biotinylated denosumab, ruthenylated denosumab, anti-RANKL antibody, 1 M Tris, pH 9.5, and soluble drug (confirmatory assay only). During this incubation, the two antigen binding sites of anti-denosumab antibodies formed a bridge between the conjugated denosumab molecules (FIG. 1A). The sample mixture was then added to a blocked MSD streptavidin standard-bind microtiter plate (MSD, #L15SA-1). The biotinylated-molecule bound to the streptavidin-coated surface of the well, resulting in the immobilization of the bridged complex. The plate was washed to remove any unbound complexes and tripropylamine GOLD read buffer (MSD, #R92TG-2) was added to each well. Using the MSD SECTOR S 600 reader, an electrical current was placed across the plate-associated electrodes. The result was a series of electrically induced oxidation-reduction reactions involving ruthenium (from the captured complex) and tripropylamine. The subsequent luminescent signal was quantified as electrochemiluminescence (ECL) units. The screening assay was performed on samples to detect the presence of binding antibodies to denosumab. Samples analyzed in the screening assay with responses below the screening assay cut point were considered negative and responses equal to or above the screening assay cut point were then analyzed in the confirmatory assay. Samples tested in the confirmatory assay with % inhibition results equal to or above the confirmatory assay cut point were considered positive for the presence of anti-denosumab antibodies.

Generation of biotinylated and ruthenylated denosumab molecules. Denosumab molecules were labeled with biotin (Thermo Scientific, #A39257) or ruthenium (MSD, #R91AO). This process included an initial buffer-exchange, a conjugation reaction, and a post-conjugation buffer-exchange to remove free label from the conjugated AMG 162 molecules.

Generation of antibodies against human RANKL. An anti-RANKL monoclonal antibody comprising murine IgG1 Fc was recombinantly expressed from HEK293-6E cells. This antibody is designated as “anti-RANKL mAb” in Examples 3-6 described below. FACS and ELISA assays using soluble RANKL confirmed that this anti-RANKL mAb binds to human RANKL. Binning assays showed that this anti-RANKL mAb binds to a different epitope bin, and does not compete with denosumab or OPG for RANKL binding.

### Example 2B. Screening and Selection of Anti-RANKL Molecules and RANKL Control

Pooled normal human serum (PNHS) was spiked with a positive control rabbit anti-denosumab polyclonal antibody (control anti-drug antibody or “ADA” for short) at low (10 ng/mL), medium (100 ng/mL), and high (1000 ng/mL) levels. These control ADA samples were then evaluated in 5 different ways (with 5 different reaction buffers): 1) with a conjugate/neutralizing mixture (untreated); 2) with a conjugate/neutralization mixture plus denosumab (confirmatory assay); 3) with a conjugate/neutralization mixture plus a murine anti-RANKL mAb (anti-RANKL1-treated); 4) with a conjugate/neutralizing mixture plus OPG (anti-RANKL2-treated); and 5) with a conjugate/neutralization mixture plus a murine anti-RANKL mAb (anti-RANKL3-treated). Three different RANKL (1250 pg/mL) controls were also prepared (RC-1, RC-2, and RC-3) and included in the experiment for evaluation and selection of the best candidate to serve as the RANKL control. The evaluation served to screen and select the optimal anti-RANKL treatment that did not impact specific ADA signal yet inhibited the specific target (RANKL) signal.

The results from the experiment are shown in Table 9. Increased (untreated) S/N results with increasing ADA concentrations were observed when the untreated conjugate/neutralizing mixture was used. Varying results were observed from the 3 different RANKL Controls (RC) with RC-1 generating results around the assay cut points, RC-2 generating weak responses that were negative in the assay, and RC-3 generating strong responses that were positive in the assay. Based on the results generated by the 3 RANKL lots, RC-3 was selected for use as the target control. Results from the second evaluation where a conjugate/neutralization mixture plus denosumab (ADA and target inhibition control) was used showed the expected inhibition (>/=60%) of both the specific ADA signal and the specific target signal, indicating that the control conditions worked as expected. Further, results from the assay where a conjugate/neutralization mixture plus anti-RANKL3 was used showed minimal inhibition of the specific ADA signal, indicating that the use of this anti-RANKL molecule did not impact the specific ADA signal. Because the anti-RANKL3 treatment did not impact the specific ADA signal and inhibited the specific target signal, the anti-RANKL3 murine antibody was considered a preferred agent among the three anti-RANKL treatment agents tested in this experiment.

TABLE 9
Result summary from anti-RANKL molecule screening

| | Reaction Buffer Composition | | | | |
|---|---|---|---|---|---|
| | | denosumab | Anti-RANKL1- | Anti-RANKL2- | Anti-RANKL3- |
| | Untreated | Treated % | Treated % | Treated % | Treated % |
| Sample | S/N | S/N Inhibition | S/N Inhibition | S/N Inhibition | S/N Inhibition |
| Negative Control (NC) | 1.00 | 0% | 0% | 0% | 0% |
| 10 ng/mL ADA | 2.34 | 60% | 58% | 8% | −4% |
| 100 ng/mL ADA | 15.39 | 94% | 89% | 14% | −3% |
| 1000 ng/mL ADA | 156.97 | 99% | 94% | 13% | −3% |
| RC-1 | 1.21 | 20% | 18% | 18% | 17% |
| RC-2 | 1.05 | 6% | 23% | 6% | 5% |
| RC-3 | 2.22 | 52% | 65% | 58% | 54% |

### Example 3. Assessing Impact of Anti-RANKL Antibody on Specific ADA Signal

Pooled normal human serum (PNHS) was spiked with a positive control rabbit anti-denosumab polyclonal antibody (anti-drug antibody or ADA) at low (10 ng/mL), medium (100 ng/mL), and high (1000 ng/mL) levels. These ADA samples were then evaluated in 3 different ways (with 3 different reaction buffers): 1) with a conjugate/neutralizing mixture (untreated); 2) with a conjugate/neutralization mixture plus anti-RANKL mAb (anti-RANKL mAb-treated); and 3) with a conjugate/neutralizing mixture plus anti-RANKL mAb and denosumab (denosumab+Anti-RANKL mAb-treated, confirmatory assay). A human RANKL (1250 pg/mL) control was also prepared and included in the experiment to confirm efficacy of the added anti-RANKL mAb at inhibiting signal generated by RANKL (target). Results generated by the RANKL control demonstrated that the assay worked as expected (data not shown). The results from the experiment are shown in Table 1A. Increased S/N results with increasing ADA concentrations were observed when the untreated conjugate/neutralizing mixture was used. Results from the assay where a conjugate/neutralization mixture plus anti-RANKL mAb was used showed minimal inhibition (<20%) of the specific anti-denosumab ADA signal, indicating that the use of the anti-RANKL mAb in the assay did not impact the specific anti-denosumab ADA signal. Results from the assay where a conjugate/neutralization mixture plus anti-RANKL mAb and denosumab were used showed a greater than 20% inhibition of the S/N results.

TABLE 1A
Impact of anti-RANKL antibody on ADA Signal

| | Reaction Buffer Composition | | | | |
|---|---|---|---|---|---|
| | | | | denosumab + | denosumab + |
| | | Anti-RANKL | Anti-RANKL | Anti-RANKL | Anti-RANKL |
| | Untreated | mAb-Treated | mAb-Treated % | mAb-Treated | mAb-Treated % |
| Sample | S/N | S/N | S/N Inhibition | S/N | S/N Inhibition |
| Negative Control (NC) | 1.00 | 1.00 | 0% | 1.00 | 0% |
| 10 ng/mL ADA | 2.06 | 1.90 | 7% | 1.03 | 50% |
| 100 ng/mL ADA | 13.08 | 11.65 | 11% | 1.05 | 92% |
| 1000 ng/mL ADA | 129.19 | 106.69 | 17% | 1.23 | 99% |

% S/N Inhibition = (Untreated S/N − Treated S/N)/Untreated S/N * 100

An additional experiment was performed where PNHS was spiked with six different positive control anti-denosumab antibodies, including polyclonal (pAb) and additional murine and rat monoclonal antibodies, at low (10 ng/mL), medium (100 ng/mL), and high (1000 ng/mL) levels. These ADA samples were then evaluated in 2 different ways (with 2 different reaction buffers): 1) with a conjugate/neutralizing mixture (untreated); and 2) with a conjugate/neutralization mixture plus anti-RANKL mAb (anti-RANKL mAb-treated). A human RANKL (1250 pg/mL) control was also prepared and included in the experiment to confirm efficacy of the added anti RANKL mAb at inhibiting signal generated by RANKL (target). Results generated by the RANKL control demonstrated that the assay worked as expected (data not shown). The results from the experiment are shown in Table 1B. Increased S/N results with increasing ADA concentrations were observed when the untreated conjugate/neutralizing mixture was used. Results from the assay where a conjugate/neutralization mixture plus anti-RANKL mAb was used showed minimal inhibition (<20%) of the specific anti-denosumab ADA signal, indicating that the use of the anti-RANKL mAb in the assay did not impact the specific anti-denosumab ADA signal.

TABLE 1B
Impact of anti-RANKL antibody on ADA Signal

| | | Reaction Buffer Composition | | |
|---|---|---|---|---|
| | | | Anti-RANKL | Anti-RANKL |
| | Positive Control | Untreated | mAb-Treated | mAb-Treated % |
| Sample | Antibody Description | S/N | S/N | S/N Inhibition |
| Negative Control (NC) | NA | 1.00 | 1.00 | 0% |
| 10 ng/mL ADA | rbt anti-denosumab | 1.41 | 1.34 | 5% |
| 100 ng/mL ADA | pAb Number 1 | 4.38 | 4.47 | −2% |
| 1000 ng/mL ADA | | 32.15 | 33.45 | −4% |
| 10 ng/mL ADA | rbt anti-denosumab | 2.29 | 2.16 | 6% |
| 100 ng/mL ADA | pAb Number 2 | 12.70 | 12.27 | 3% |
| 1000 ng/mL ADA | | 112.58 | 113.32 | −1% |
| 10 ng/mL ADA | rbt anti-denosumab | 2.78 | 2.69 | 3% |
| 100 ng/mL ADA | pAb Number 3 | 18.58 | 18.38 | 1% |
| 1000 ng/mL ADA | | 163.56 | 169.21 | −3% |
| 10 ng/mL ADA | rbt anti-denosumab | 1.80 | 1.67 | 7% |
| 100 ng/mL ADA | pAb Number 4 | 8.15 | 8.56 | −5% |
| 1000 ng/mL ADA | | 67.29 | 71.30 | −6% |
| 10 ng/mL ADA | mu anti-denosumab | 6.21 | 6.33 | −2% |
| 100 ng/mL ADA | 2.5.1 mAb | 50.97 | 54.34 | −7% |
| 1000 ng/mL ADA | | 447.25 | 466.03 | −4% |
| 10 ng/mL ADA | rat anti-denosumab | 3.20 | 3.45 | −8% |
| 100 ng/mL ADA | 3.12 mAb | 23.37 | 24.98 | −7% |
| 1000 ng/mL ADA | | 222.09 | 242.39 | −9% |

% S/N Inhibition = (Untreated S/N − Treated S/N)/Untreated S/N * 100

These results further demonstrated that the detection of ADA was not impacted when an anti-RANKL mAb was incorporated into the assay.

### Example 4. Assessing Impact of Anti-RANKL Antibody on Specific RANKL Signal

PNHS was spiked with human RANKL at various levels starting with low levels previously shown to generate a low positive (1250 pg/mL), medium (2500 pg/mL), and high (5000 pg/mL) levels. The RANKL-spiked samples were then evaluated in 3 different ways (with 3 different reaction buffers): 1) with a conjugate/neutralizing mixture (untreated); 2) with a conjugate/neutralization mixture plus anti-RANKL mAb; and 3) with a conjugate/neutralizing mixture plus anti-RANKL mAb and denosumab (confirmatory assay).

The results from the experiment are shown in Table 2. Increased S/N results with increasing RANKL concentrations were observed when the untreated conjugate/neutralizing mixture was used. Results from the experiment where a conjugate/neutralization mixture plus anti-RANKL mAb was used showed inhibition (>20%) of the specific RANKL signal at all concentrations tested, indicating that the use of the anti-RANKL mAb in the assay inhibited signal generated by target. Results from the assay where a conjugate/neutralization mixture plus anti-RANKL mAb and denosumab were used also showed a greater than 20% inhibition of the S/N results.

TABLE 2
Impact of anti-RANKL antibody on RANKL signal

| | Reaction Buffer Composition | | | | |
|---|---|---|---|---|---|
| | | | | AMG 162 + | AMG 162 + |
| | | Anti-RANKL | Anti-RANKL | Anti-RANKL | Anti-RANKL |
| | Untreated | mAb-Treated | mAb-Treated % | mAb-Treated | mAb-Treated % |
| Sample | S/N | S/N | S/N Inhibition | S/N | S/N Inhibition |
| Negative Control (NC) | 1.00 | 1.00 | 0% | 1.00 | 0% |
| 1250 pg/mL huRANKL | 1.94 | 0.99 | 49% | 1.04 | 46% |
| 2500 pg/mL huRANKL | 3.01 | 1.15 | 62% | 1.00 | 67% |
| 5000 pg/mL huRANKL | 4.86 | 1.33 | 73% | 0.93 | 81% |

% S/N Inhibition = (Untreated S/N − Treated S/N)/Untreated S/N * 100

These combined results demonstrated that detection of RANKL was inhibited when an anti-RANKL mAb was incorporated into the assay.

### Example 5. Assessing Impact of Anti-RANKL Antibody on Samples Containing Both ADA and RANKL

In order to simulate study samples, which may contain both ADA and RANKL, PNHS was spiked with low (10 ng/mL) or medium (100 ng/mL) concentrations of ADA and low (1250 pg/mL), medium (2500 pg/mL), and high (5000 pg/mL) concentrations of RANKL. The combination samples were then evaluated in 3 different ways (with 3 different reaction buffers): 1) with a conjugate/neutralizing mixture (untreated); 2) with a conjugate/neutralization mixture plus anti-RANKL mAb; and 3) with a conjugate/neutralizing mixture plus anti-RANKL mAb and denosumab (confirmatory assay).

The results from the experiment are shown in Table 3A. Increasing S/N values with increasing ADA and/or RANKL concentrations were observed when the untreated conjugate/neutralizing mixture was used. Results from the experiment where a conjugate/neutralization mixture plus anti-RANKL mAb was used showed that a portion of the signal was inhibited, indicating that the specific ADA signal was not inhibited. Results from the assay where a conjugate/neutralization mixture plus anti-RANKL mAb and denosumab were used showed inhibition of the signal (>20%) at all concentrations tested.

TABLE 3A
Impact of anti-RANKL antibody on samples containing both ADA and RANKL

| | Reaction Buffer Composition | | | | |
|---|---|---|---|---|---|
| | | | | AMG 162 + | AMG 162 + |
| | | Anti-RANKL | Anti-RANKL | Anti-RANKL | Anti-RANKL |
| | Untreated | mAb-Treated | mAb-Treated % | mAb-Treated | mAb-Treated % |
| Sample | S/N | S/N | S/N Inhibition | S/N | S/N Inhibition |
| Negative Control (NC) | 1.00 | 1.00 | 0% | 1.00 | 0% |
| 10 ng/mL ADA/ | 2.94 | 1.92 | 35% | 1.03 | 65% |
| 1250 pg/mL RANKL | | | | | |
| 10 ng/mL ADA/ | 4.01 | 1.95 | 51% | 1.05 | 74% |
| 2500 pg/mL RANKL | | | | | |
| 10 ng/mL ADA/ | 5.69 | 2.00 | 65% | 0.97 | 83% |
| 5000 pg/mL RANKL | | | | | |
| 100 ng/mL ADA/ | 13.58 | 11.49 | 15% | 1.05 | 92% |
| 1250 pg/mL RANKL | | | | | |
| 100 ng/mL ADA/ | 16.23 | 11.67 | 28% | 1.11 | 93% |
| 2500 pg/mL RANKL | | | | | |
| 100 ng/mL ADA/ | 18.13 | 11.20 | 38% | 1.11 | 94% |
| 5000 pg/mL RANKL | | | | | |

% S/N Inhibition = (Untreated S/N − Treated S/N)/Untreated S/N * 100

Table 3B shows the S/N results of the combination samples compared to the S/N results of samples containing ADA only (no RANKL) at the same concentrations. At both ADA concentrations (and increasing RANKL concentrations), an increase in S/N was observed. Results from the experiment where the untreated conjugate/neutralization mixture plus anti-RANKL mAb was used showed S/N results consistent with those generated by the ADA-only samples at the same concentrations, indicating that the anti-RANKL mAb was successful at inhibiting the RANKL-specific signal without impacting the specific ADA signal.

TABLE 3B
Impact of anti-RANKL antibody on samples containing both
ADA and RANKL compared to samples containing ADA only

| | | | | | % CV of S/N Results of |
|---|---|---|---|---|---|
| Sample Composition | | Reaction Buffer Composition | | % | Untreated and Anti- |
| ADA | RANKL | | Anti-RANKL | Recovery | RANKL mAb-Treated |
| Concentration | Concentration | Untreated | mAb-Treated | of ADA | Samples containing ADA |
| (ng/mL) | (pg/mL) | S/N | S/N | Signal | or ADA + RANKL |
| 10 | 0 | 2.06 | 1.90 | 92% | 3% |
| | 1250 | 2.94 | 1.92 | 93% | |
| | 2500 | 4.01 | 1.95 | 95% | |
| | 5000 | 5.69 | 2.00 | 97% | |
| 100 | 0 | 13.08 | 11.65 | 89% | 6% |
| | 1250 | 13.58 | 11.49 | 88% | |
| | 2500 | 16.23 | 11.67 | 89% | |
| | 5000 | 18.13 | 11.20 | 86% | |

% Recovery = (Untreated S/N + Treated S/N) * 100

These results demonstrated that the detection of ADA was not impacted and that the detection of RANKL was inhibited when an anti-RANKL mAb was incorporated into the assay.

As denosumab binds to human RANKL with high affinity, soluble RANKL that may be present in human serum samples may interfere (cause false negative results) or cross-react (cause false positive results in the assay). In order to minimize possible interference or cross-reactivity by RANKL, a human-murine chimeric anti-RANKL monoclonal antibody that competes with denosumab for binding to RANKL was incorporated into the assay.

### Example 6. Characterization of Anti-RANKL Antibodies

ELISA assays. Soluble RANKL (sRANKL) at 10 ug/ml in 1×PBS/0.05% azide (50 ul/well) was used to coat on Costar 3368 medium binding 96 well plate; samples were incubated overnight 4° C. Plates were washed using Titertek with RO (reverse osmosis) water with 3-cycle wash; then blocked with 250 ul of 1×PBS/1% milk, incubated at least 30 min RT; then washed using Titertek with RO water with 3-cycle wash again. Primary antibodies were purified mAbs clones, titrated 1:3 from 1 ug/ml 1×PBS/1% milk (50 ul/well), and incubated 1 hour at RT. Positive control 1 (dmab-IgG1) was a modified denosumab where the original IgG2 Fc region of denosumab was replaced with IgG1 Fc region. IgG1 Fc was more convenient for detection. Positive control 2 was OPG-Fc titrated 1:3 from 1 ug/ml. After incubating with primary antibodies, the plates were washed using Titertek with RO water 3-cycle wash, then incubated with secondary antibody. Secondary antibody was goat anti-human IgG Fc (cross-adsorbed)-HRP (Thermo Fisher, 31413) at 0.4 ug/ml, in 1×PBS/1% milk (50 ul/well), incubated 1 hour at RT. Samples were then washed using Titertek with RO water 3-cycle wash. Substrate was treated with 1 step TMB (Neogen, 308177) (50 ul/well) quenched with 1N Hydrochloric acid (50 ul/well) after 30 minutes at RT. Plates were read at 450 nm, and the results were shown in Tables 4.

TABLE 4
Soluble human RANKL Binding Data

| Purified | sHuman RANK-L @ 10 ug/mL | | | | | | | |
|---|---|---|---|---|---|---|---|---|
| Mabs\Conc. | 1 ug/ml | 0.333 | 0.111 | 0.037 | 0.012 | 0.004 | 0.001 | 0 |
| anti-RANKL | 5.1 | 4.4 | 4.0 | 2.6 | 1.5 | 0.7 | 0.4 | 0.2 |
| mAb | | | | | | | | |
| Positive | 6.0 | 6.0 | 6.0 | 6.0 | 3.0 | 1.5 | 0.7 | 0.2 |
| control 1 | | | | | | | | |
| OPG-Fc | 6.0 | 5.5 | 5.0 | 4.5 | 3.2 | 1.6 | 0.6 | 0.2 |
| Neg control 1 | 0.3 | 0.3 | 0.3 | 0.3 | 1.5 | 0.3 | 0.23 | 0.3 |
| Neg control 2 | 0.3 | 0.3 | 0.3 | 0.3 | 0.3 | 0.2 | 0.2 | 0.2 |

FACS assays. Standard FACS protocols are as follows: Ren 218.9 stable cells (CHO/RANKL) were counted and seeded in FACS buffer (1×PBS+2% Fetal Bovine Serum) such that there were 50,000 cells/well in 50 uls/well final volume. Cells and primary antibody at 5 ug/mL and 0.25 ug/mL were incubated 1 hour at 4° C., then washed 2× with 200 uls/well FACS buffer. 100 uls/well of secondary antibody mixed with dead cell stain 7-Aminoactinomycin D (7AAD) (Sigma, A9400-5MG) were then added. Goat anti human IgG Cy5 secondary antibody (Jackson, 109-175-098) and 7AAD were both used at 5.0 ug/mL and were incubated with the samples for 15 minutes at 4° C. and then washed 1× with 200 uls/well FACS buffer. Cells were then resuspended in 100 ul FACS buffer and read by flow cytometry with high throughput screening (HTS) head.

TABLE 5
FACS binding Data

Conc.

| | mAb @ 5 ug/mL | mAb @ 0.25 ug/mL |
|---|---|---|
| Purified Mabs | X Geo Mean | X Geo Mean |
| anti-RANKL mAb | 5353.4 | 1981.38 |
| Positive control 1 | 5567.8 | 3393.54 |
| Neg control 1 | 4.98 | 4.92 |
| Neg control 2 | 4.49 | 4.74 |

Additional ELISA assays with human mouse and rat soluble RANKL. An ELISA protocol was used that was nearly identical to ELISA assays described above, except the human, mouse and rat were photobiotinylated (pb) and captured onto neutravidin (NA). Tables 6-7 show results of ELISA assays using human, mouse, and rat RANKL.

TABLE 6
Soluble human and mouse RANKL Binding Data

| Purified | Human RANK-L @ 250 ng/mL | | | | Mouse RANKL | | | |
|---|---|---|---|---|---|---|---|---|
| Mabs\ | 1 | | | | 1 | | | |
| Conc. | ug/ml | 0.1 | 0.01 | 0.001 | ug/ml | 0.1 | 0.01 | 0.001 |
| anti- | 4.710 | 3.525 | 1.946 | 0.264 | 0.064 | 0.061 | 0.055 | 0.057 |
| RANKL | | | | | | | | |
| mAb | | | | | | | | |

TABLE 7
Soluble rat RANKL Binding Data

Conc.
Rat RANK-L @ 250 ng/mL

| Purified Mabs | 1 ug/ml | 0.1 | 0.01 | 0.001 |
|---|---|---|---|---|
| anti-RANKL mAb | 0.079 | 0.067 | 0.068 | 0.070 |

Table 8 summarizes the characteristics of certain anti-RANKL antibodies disclosed herein.

TABLE 8
Characteristics of antibodies to human RANKL

| Antibody | Immunogen | | Neutralizing | Epitope |
|---|---|---|---|---|
| H1 | human RANKL | monoclonal | Yes | DE loop |
| | 143-317 | | | |
| Denosumab | human RANKL | monoclonal | Yes | DE loop |
| | on CHO | | | |
| C1 | rec. human | monoclonal | Yes | DE loop |
| | RANKL | | | |
| A1 | rec. human | monoclonal | Yes | DE loop |
| | RANKL or human | | | |
| | RANKL on CHO | | | |
| B1 | rec. human | monoclonal | Yes | DE loop |
| | RANKL | | | |
| F1 | rec. human | monoclonal | Yes | ND |
| | RANKL | | | |
| E1 | human RANKL | monoclonal | Yes | ND |
| | on CHO | | | |
| D1 | rec. human | monoclonal | Yes | DE loop |
| | RANKL | | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### Sequence Tables

TABLE A
Sequence ID numbers for CDRs (AA)

| Antibody | Heavy Chain (HC) | | | Light Chain (LC) | | |
|---|---|---|---|---|---|---|
| Name | CDR1 | CDR2 | CDR3 | CDR1 | CDR2 | CDR3 |
| A1 | 1 | 2 | 3 | 4 | 5 | 6 |
| B1 | 7 | 8 | 9 | 10 | 11 | 12 |
| C1 | 13 | 14 | 15 | 16 | 17 | 18 |
| D1 | 19 | 20 | 21 | 22 | 23 | 24 |
| E1 | 25 | 26 | 27 | 28 | 29 | 30 |
| F1 | 31 | 32 | 33 | 34 | 35 | 36 |
| G1 | 37 | 38 | 39 | 40 | 41 | 42 |
| H1 | 43 | 44 | 45 | 46 | 47 | 48 |
| I1 | 49 | 50 | 51 | 52 | 53 | 54 |
| J1 | 55 | 56 | 57 | 58 | 59 | 60 |
| K1 | 61 | 62 | 63 | 64 | 65 | 66 |
| L1 | 67 | 68 | 69 | 70 | 71 | 72 |
| M1 | 73 | 74 | 75 | 76 | 77 | 78 |
| N1 | 79 | 80 | 81 | 82 | 83 | 84 |
| O1 | 85 | 86 | 87 | 88 | 89 | 90 |
| P1 | 91 | 92 | 93 | 94 | 95 | 96 |
| Q1 | 97 | 98 | 99 | 100 | 101 | 102 |
| R1 | 103 | 104 | 105 | 106 | 107 | 108 |
| S1 | 109 | 110 | 111 | 112 | 113 | 114 |
| T1 | 115 | 116 | 117 | 118 | 119 | 120 |
| U1 | 121 | 122 | 123 | 124 | 125 | 126 |

TABLE B
Sequence ID numbers for Variable Regions (AA)

| Antibody Name | HC variable (VH) | LC variable (VL) |
|---|---|---|
| A1 | 127 | 128 |
| B1 | 129 | 130 |
| C1 | 131 | 132 |
| D1 | 133 | 134 |
| E1 | 135 | 136 |
| F1 | 137 | 138 |
| G1 | 139 | 140 |
| H1 | 141 | 142 |
| I1 | 143 | 144 |
| J1 | 145 | 146 |
| K1 | 147 | 148 |
| L1 | 149 | 150 |
| M1 | 151 | 152 |
| N1 | 153 | 154 |
| O1 | 155 | 156 |
| P1 | 157 | 158 |
| Q1 | 159 | 160 |
| R1 | 161 | 162 |
| S1 | 163 | 164 |
| T1 | 165 | 166 |
| U1 | 167 | 168 |

TABLE C
CDR sequences

| SEQ ID | | |
|---|---|---|
| NO. | Name | Sequence |
| 1 | A1_VH CDR1 | SNGMH |
| 2 | A1_VH CDR2 | GIGTAGGTYYADSVKG |
| 3 | A1_VH CDR3 | KNWGWFDP |
| 4 | A1_VL CDR1 | RASQSVNSYLA |
| 5 | A1_VL CDR2 | DASNRAT |
| 6 | A1_VL CDR3 | QQRSNWPPFT |
| 7 | B1_VH CDR1 | SNGMH |
| 8 | B1_VH CDR2 | GIGTAGGTYYADSVKG |
| 9 | B1_VH CDR3 | KDWGWFDP |
| 10 | B1_VL CDR1 | RASQSISSYLA |
| 11 | B1_VL CDR2 | DASNRAT |
| 12 | B1_VL CDR3 | QQRSKWPPYT |
| 13 | C1_VH CDR1 | SYGMH |
| 14 | C1_VH CDR2 | GIGTGGGTYYADSVKG |
| 15 | C1_VH CDR3 | KNWGWFDY |
| 16 | C1_VL CDR1 | RASQSISSYLA |
| 17 | C1_VL CDR2 | DASNRAT |
| 18 | C1_VL CDR3 | QQRSKWPPYT |
| 19 | D1_VH CDR1 | GHALH |
| 20 | D1_VH CDR2 | GIGTHGGTYYADSVKG |
| 21 | D1_VH CDR3 | RNWGQFDY |
| 22 | D1_VL CDR1 | RASQSVSSYLA |
| 23 | D1_VL CDR2 | DASNRAT |
| 24 | D1_VL CDR3 | QQRSNWPPYT |
| 25 | E1_VH CDR1 | AYPMH |
| 26 | E1_VH CDR2 | GIGSGGGTNYADSVKG |
| 27 | E1_VH CDR3 | GRNSFDY |
| 28 | E1_VL CDR1 | RASQGISTWLA |
| 29 | E1_VL CDR2 | AASSLQS |
| 30 | E1_VL CDR3 | QQYNSYPPT |
| 31 | F1_VH CDR1 | HYPLH |
| 32 | F1_VH CDR2 | GIHTGGGTYYTDSVKG |
| 33 | F1_VH CDR3 | GRNSFDY |
| 34 | F1_VL CDR1 | RASQGISSWLA |
| 35 | F1_VL CDR2 | AASSLQS |
| 36 | F1_VL CDR3 | QQYNSYPPT |
| 37 | G1_VH CDR1 | RYAMN |
| 38 | G1_VH CDR2 | GISTSGGSTYYADSVKG |
| 39 | G1_VH CDR3 | DGDRGVIMTVEDY |
| 40 | G1_VL CDR1 | RASQSVRSSYLA |
| 41 | G1_VL CDR2 | GASSRAT |
| 42 | G1_VL CDR3 | QQYGSSPRT |
| 43 | H1_VH CDR1 | NYAIH |
| 44 | H1_VH CDR2 | WINAGNGNTKFSQKFQG |
| 45 | H1_VH CDR3 | DSSNMVRGIIIAYYFDY |
| 46 | H1_VL CDR1 | RASQSISRYLN |
| 47 | H1_VL CDR2 | GASSLQS |
| 48 | H1_VL CDR3 | QHTRA |
| 49 | I1_VH CDR1 | NYAIH |
| 50 | I1_VH CDR2 | WINAGNGNTKFSQKFQG |
| 51 | I1_VH CDR3 | DSSNMVRGIIIAYYFDY |
| 52 | Il_VL CDR1 | RASQSVGSYLA |
| 53 | I1_VL CDR2 | DATNRAT |
| 54 | I1_VL CDR3 | QHRRT |
| 55 | J1_VH CDR1 | DYAMH |
| 56 | J1_VH CDR2 | GISWNSGRIGYADSVKG |
| 57 | J1_VH CDR3 | GGSTSARYSSGWYY |
| 58 | J1_VL CDR1 | SGDALPKQYVY |
| 59 | J1_VL CDR2 | EDSERPS |
| 60 | J1_VL CDR3 | QSTDSSGTYVV |
| 61 | K1_VH CDR1 | NYPMH |
| 62 | K1_VH CDR2 | VISYDGNNKYYADSVKG |
| 63 | K1_VH CDR3 | GGGGFDY |
| 64 | K1_VL CDR1 | RASQSVSSSSLA |
| 65 | K1_VL CDR2 | GASSRAT |
| 66 | K1_VL CDR3 | QQYGA |
| 67 | L1_VH CDR1 | NYWMS |
| 68 | L1_VH CDR2 | SISSSSSYIYYADSVKG |
| 69 | L1_VH CDR3 | QSESSVYGMDV |
| 70 | L1_VL CDR1 | SGSHSNIASSSAN |
| 71 | L1_VL CDR2 | GNDQRPS |
| 72 | L1_VL CDR3 | AAWDDSLSGVV |
| 73 | M1_VH CDR1 | NAWMS |
| 74 | M1_VH | SISSSSSYIYYADSVKG |
| | CDR2 | |
| 75 | M1_VH CDR3 | DGSIFGVVTPFDY |
| 76 | M1_VL CDR1 | TGSSSNIGAGYDVH |
| 77 | M1_VL | SNNQRPS |
| | CDR2 | |
| 78 | M1_VL | AAWDDSLRGPHWV |
| | CDR3 | |
| 79 | N1_VH CDR1 | NTWMN |
| 80 | N1_VH CDR2 | SISVNSNYIYYADSVKG |
| 81 | N1_VH CDR3 | DSEYAFQN |
| 82 | N1_VL CDR1 | TGSSSNIGAGYDVH |
| 83 | N1_VL CDR2 | DNEWRPS |
| 84 | N1_VL CDR3 | ATWDDSLHWV |
| 85 | O1_VH CDR1 | DHWMS |
| 86 | O1_VH CDR2 | SISSSSSYIYYADSVKG |
| 87 | O1_VH CDR3 | HEDTYYYYGMDV |
| 88 | O1_VL CDR1 | SGSFSNIGGNTVN |
| 89 | O1_VL CDR2 | DNDQRPS |
| 90 | O1_VL CDR3 | ASWDDSLSGVV |
| 91 | P1_VH CDR1 | KDWMS |
| 92 | P1_VH CDR2 | VISYDGSNKYYADSVKG |
| 93 | P1_VH CDR3 | AAPHYYYGLDV |
| 94 | P1_VL CDR1 | SGSSSNIGSNTVN |
| 95 | P1_VL CDR2 | YDDLLPS |
| 96 | P1_VL CDR3 | GSWDSSLSAVV |
| 97 | Q1_VH CDR1 | GFGMH |
| 98 | Q1_VH CDR2 | VISYDGRNKYYADSVKG |
| 99 | Q1_VH CDR3 | SLSGTYWGYGMDV |
| 100 | Q1_VL CDR1 | RASQSIRSWLA |
| 101 | Q1_VL CDR2 | KASSLES |
| 102 | Q1_VL CDR3 | QQYNSYLWT |
| 103 | R1_VH CDR1 | GYGIH |
| 104 | R1_VH CDR2 | VISYDGTNKYYGDSVKG |
| 105 | R1_VH CDR3 | SLSGTYWGYGMDV |
| 106 | R1_VL CDR1 | RASQNIGNWLA |
| 107 | R1_VL CDR2 | KASSLQN |
| 108 | R1_VL CDR3 | QRYNSYSWT |
| 109 | S1_VH CDR1 | GYGMH |
| 110 | S1_VH CDR2 | VISYDGSNKHYADSVKG |
| 111 | S1_VH CDR3 | SLSGTYWGYGMDV |
| 112 | S1_VL CDR1 | RASQNISSWLA |
| 113 | S1_VL CDR2 | KASSLES |
| 114 | S1_VL CDR3 | QQYNRYSWT |
| 115 | T1_VH CDR1 | NYYIE |
| 116 | T1_VH CDR2 | VINPGWGDTNYNEKFKG |
| 117 | T1_VH CDR3 | RDTAHGYYALDP |
| 118 | T1_VL CDR1 | KASQNVGTNVA |
| 119 | T1_VL CDR2 | SASYRYS |
| 120 | T1_VL CDR3 | QQYWDYPLT |
| 121 | U1_VH CDR1 | NYYIE |
| 122 | U1_VH CDR2 | VINPGWGDTNYNEKFKG |
| 123 | U1_VH CDR3 | RDTRHGYYALDY |
| 124 | U1_VL CDR1 | KASQNVGTNVA |
| 125 | U1_VL CDR2 | SASYRYS |
| 126 | U1_VL CDR3 | QQYNDYPLT |

TABLE D
VH VL sequences

| SEQ | | |
|---|---|---|
| ID NO | Name | Sequence |
| 127 | A1 VH | EVQLVQSGGGLVHPGGSLRLSCEGSGFTFSSNGMHWVRQTPGKGLEWVSGIGTAGGTYYADSVKG |
| | | RFTISRDNVKKSLYLQMNSLRAEDMAIYYCVRKNWGWFDPWGQGALVTVSS |
| 128 | A1 VL | EIVLTQSPATLSLSPGERATLSCRASQSVNSYLAWFQQKPGQAPRLLIYDASNRATGIPARFSGS |
| | | GSGTDFTLTISSLEPEDFAIYYCQQRSNWPPFTFGQGTKLEIKR |
| 129 | B1 VH | EVQLVQSGGGLVHPGGSLRLSCEGSGFTFSSNGMHWVRQTPGKGLEWVSGIGTAGGTYYADSVKG |
| | | RFTISRDNVKKSLYLQMNSLRAEDMAIYYCVRKDWGWFDPWGQGALVTVSS |
| 130 | B1 VL | EIVLTQSPATLSLSPGERATLSCRASQSISSYLAWYQQKPGQAPRLLIYDASNRATGIPARFSGS |
| | | GSGTDFTLTISSLEPEDFAVYYCQQRSKWPPYTFGQGTKLEIKR |
| 131 | C1 VH | EVQLVQSGGGLVHPGGSLRLSCAGSGFTFSSYGMHWVRQAPGKGLEWVSGIGTGGGTYYADSVKG |
| | | RFTISRDNVKNSLYLQMNSLRAEDMAVYYCARKNWGWFDYWGQGTLVTVSS |
| 132 | C1 VL | EIVLTQSPATLSLSPGERATLSCRASQSISSYLAWYQQKPGQAPRLLIYDASNRATGIPARFSGS |
| | | GSGTDFTLTISSLEPEDFAVYYCQQRSKWPPYTFGQGTKLEIKR |
| 133 | D1 VH | EVQLVQSGGGLVHPGGSLRLSCAGSGFTFSGHALHWVRQAPGKGLEWVSGIGTHGGTYYADSVKG |
| | | RFTISRDNAKNSLFLQMNSLSAEDMAVYYCTRRNWGQFDYWGQGTLVTVSS |
| 134 | D1 VL | EIVLTQSPATLSLSPGERATLSCRASQSVSSYLAWYQQKPGQAPRLLIYDASNRATGIPARFSGS |
| | | GSGTDFTLTISSLEPEDFAVYYCQQRSNWPPYTFGQGTKLEIKR |
| 135 | E1 VH | EVQLVQSGGGLVHPGGSLRLSCVGSRFTFSAYPMHWVRQAPGKGLEWVSGIGSGGGTNYADSVKG |
| | | RFTISRDTAKNSLYLQMNSLRAEDMAVYYCARGRNSFDYWGQGTLVTVSS |
| 136 | E1 VL | DIQMTQSPSSLSASVGDRVTITCRASQGISTWLAWYQQKPEKAPKSLIYAASSLQSGVPSRFSGS |
| | | GSGTDFTLTISSLQPEDFATYYCQQYNSYPPTFGQGTKVEIKR |
| 137 | F1 VH | EVQLVQSGGDLVHPGGSLRLSCVGSGFTFSHYPLHWVRQAPGKGLEWISGIHTGGGTYYTDSVKG |
| | | RFTISSDNAKNSLYLQMNTLRAEDMAVYYCARGRNSFDYWGQGTLVIVSS |
| 138 | F1 VL | DIQMTQSPSSLSASVGDRVTITCRASQGISSWLAWYQQKPEKAPKSLIYAASSLQSGVPSRFSGS |
| | | GSGTDFTLTISSLQSEDFATYYCQQYNSYPPTFGQGTRLEIKR |
| 139 | G1 VH | EVHLLESGGGLVQPGGSLRLSCAASGFTFSRYAMNWVRQAPGKGLEWVSGISTSGGSTYYADSVK |
| | | GRFTISRDISRNTLNLQMNSLRAEDTAVYYCAKDGDRGVIMTVFDYWGQGTLVTVSS |
| 140 | G1 VL | EIVLTQSPGTLSLSPGERATLSCRASQSVRSSYLAWYQQKPGQAPRLLIYGASSRATGIPDRFSG |
| | | SGSGTDFTLTISRLEPEDFAVYYCQQYGSSPRTFGQGTKVEIKR |
| 141 | H1 VH | QVQLVQSGAEVRKPGASVKVSCKASGYDFSNYAIHWVRQAPGQRLEWMGWINAGNGNTKFSQKFQ |
| | | GRITVTRDTAASTAYMELRSLRSEDTAVYYCARDSSNMVRGIIIAYYFDYWGQGTLVTVSS |
| 142 | H1 VL | EIVMTQSPSSLSASVGDRVTITCRASQSISRYLNWYQLKPGKAPRLLIYGASSLQSGVPSRFSGS |
| | | GSGAEFTLTISSLQPEDIATYYCQHTRAFGQGTKVEIKR |
| 143 | I1 VH | EVQLVQSGAEVRKPGASVKVSCKASGYDFSNYAIHWVRQAPGQRLEWMGWINAGNGNTKFSQKFQ |
| | | GRITVTRDTAASTAYMELRSLRSEDTAVYYCARDSSNMVRGIIIAYYFDYWGQGTLVTVSS |
| 144 | I1 VL | EIVLTQSPATLSFSPGERATLSCRASQSVGSYLAWYQQRPGQAPRPLIYDATNRATGIPTRFSGS |
| | | GSGTDFTLTISSLEPEDFATYYCQHRRTFGRGTKLEIKR |
| 145 | J1 VH | EVQLLESGGGLVQPGRSLRLSCAASGFTEDDYAMHWVRQAPGKGLEWVSGISWNSGRIGYADSVK |
| | | GRFTISRDNAKNSLYLQMNSLRPEDTAFYYCAKGGSTSARYSSGWYYWGQGTLVTVSS |
| 146 | J1 VL | QSVLTQPPSVSVSPGQTATITCSGDALPKQYVYWYRQKPGQAPLLVIYEDSERPSGIPERFSGSS |
| | | SGTEVTLSISGVQAEDEADYYCQSTDSSGTYVVFGGGTKLTVLS |
| 147 | K1 VH | EVQLVQSGGGLVQPGGSLRLSCLVSGFTFNNYPMHWVRQAPGKGLEWVAVISYDGNNKYYADSVK |
| | | GRFTISRDNSKNTLYLQMNSLRSEDTAVYYCARGGGGFDYWGQGTLVTVSS |
| 148 | K1 VL | EIVMTQSPGTLSLSPGERATLSCRASQSVSSSSLAWYQQKPGQAPRLLIYGASSRATGIPDRFSG |
| | | SGSGTDFTLTISRLEPEDFAVYYCQQYGAFGGGTKVEIKR |
| 149 | L1 VH | EVQLLESGGGLVQPGGSLRLSCAASGFTFSNYWMSWVRQAPGKGLEWVSSISSSSSYIYYADSVK |
| | | GRFTISRDNSKNTLYLQMNSLRAEDTAVYYCARQSESSVYGMDVWGQGTLVTVSS |
| 150 | L1 VL | QSVLTQPPSASGTPGQRVTISCSGSHSNIASSSANWYQQLPGTAPKLLIYGNDQRPSGVPDRFSG |
| | | SKSGTSASLAISGLRSEDEADYYCAAWDDSLSGVVFGGGTKLTVLGQP |
| 151 | M1 VH | EVQLLESGGGLVQPGGSLRLSCAASGFTFSNAWMSWVRQAPGKGLEWVSSISSSSSYIYYADSVK |
| | | GRFTISRDNSKNTLYLQMNSLRAEDTAVYYCARDGSIFGVVTPFDYWGQGTLVTVSS |
| 152 | M1 VL | QSVLTQPPSASGTPGQRVTISCTGSSSNIGAGYDVHWYQQLPGTAPKLLIYSNNQRPSGVPDRFS |
| | | GSKSGTSASLAISGLRSEDEADYYCAAWDDSLRGPHWVFGGGTKLTVLGQP |
| 153 | N1 VH | EVQLLESGGGLVQPGGSLRLSCAASGFTFSNTWMNWVRQAPGKGLEWVSSISVNSNYIYYADSVK |
| | | GRFTISRDNSKNTLYLQMNSLRAEDTAVYYCARDSEYAFQNWGQGTLVTVSS |
| 154 | N1 VL | QSVLTQPPSASGTPGQRVTISCTGSSSNIGAGYDVHWYQQLPGTAPKLLIYDNEWRPSGVPDRFS |
| | | GSKSGTSASLAISGLRSEDEADYYCATWDDSLHWVFGGGTKLTVLGQP |
| 155 | 01 VH | EVQLLESGGGLVQPGGSLRLSCAASGFTFSDHWMSWVRQAPGKGLEWVSSISSSSSYIYYADSVK |
| | | GRFTISRDNSKNTLYLQMNSLRAEDTAVYYCARHEDTYYYYGMDVWGQGTLVTVSS |
| 156 | 01 VL | QSVLTQPPSASGTPGQRVTISCSGSFSNIGGNTVNWYQQLPGTAPKLLIYDNDQRPSGVPDRFSG |
| | | SKSGTSASLAISGLRSEDEADYYCASWDDSLSGVVFGGGTKLTVLGQP |
| 157 | P1 VH | EVQLLESGGGLVQPGGSLRLSCAASGFTFRKDWMSWVRQAPGKGLEWVAVISYDGSNKYYADSVK |
| | | GRFTISRDNSKNTLYLQMNSLRAEDTAVYYCTRAAPHYYYGLDVWGQGTLVTVSS |
| 158 | P1 VL | QSVLTQPPSASGTPGQRVTISCSGSSSNIGSNTVNWYQQLPGTAPKLLIYYDDLLPSGVPDRFSG |
| | | SKSGTSASLAISGPRSEDEADYYCGSWDSSLSAVVFGGGTKLTVLGQP |
| 159 | Q1 VH | QVQLVESGGGVVQPGRSLRLSCAASGFTFSGFGMHWVRQAPGKGLEWVAVISYDGRNKYYADSVK |
| | | GRFTISRDNSKNTLYLQMNSLRAEDTAVYYCAKSLSGTYWGYGMDVWGQGTTVTVS |
| 160 | Q1 VL | DIQMTQSPSTLSASVGDRVTITCRASQSIRSWLAWYQQKPGKAPKLLIYKASSLESGVPSRFSGS |
| | | GSGTEFTLTISSLQPDDFATYYCQQYNSYLWTFGQGTKVEIK |
| 161 | R1 VH | QVQLVESGGGVVQPGRSLRLSCAASGFTFSGYGIHWVRQAPDKGLEWVAVISYDGTNKYYGDSVK |
| | | GRFTISRDNTKNTLYLQMNSLRTEDTAVYYCAKSLSGTYWGYGMDVWGQGTTVTVS |
| 162 | R1 VL | DIQMTQSPSTLSASVGDRVTITCRASQNIGNWLAWYQQKPGKAPKLLIYKASSLQNGVPSRFSGS |
| | | GSGTEFTLTISSLQPDDFATYYCQRYNSYSWTFGQGTKVEIK |
| 163 | S1 VH | QVQLVESGGGVVQPGRSLRLSCAASGFTFSGYGMHWVRQAPGKGLDWVTVISYDGSNKHYADSVK |
| | | GRFTISRDNSKNTLYLQMSSLGPEDTAVYYCAKSLSGTYWGYGMDVWGQGTTVTVS |
| 164 | S1 VL | DIQMTQSPSTLSASVGDRVTITCRASQNISSWLAWYQQKPGKAPKLLIYKASSLESGVPSRFSGS |
| | | GSGTEFTLTVSSLQPDDFATYYCQQYNRYSWTFGQGTKVEIK |
| 165 | T1 VH | QVQLVQSGAEVKKPGSSVKVSCKASGYAFTNYYIEWVRQAPGQGLEWMGVINPGWGDTNYNEKFK |
| | | GRVTITADKSTSTAYMELSSLRSEDTAVYYCARRDTAHGYYALDPWGQGTTVTVSS |
| 166 | T1 VL | DIQMTQSPSSLSASVGDRVTITCKASQNVGTNVAWYQQKPGKAPKLLIYSASYRYSGVPSRFSGS |
| | | GSGTDFTLTISSLQPEDFATYYCQQYWDYPLTFGGGTKVEIK |
| 167 | U1 VH | QVQLQQSGAELVRPGTSVKVSCKASGYAFTNYYIEWLKQRPGQGLEWIGVINPGWGDTNYNEKFK |
| | | GKATLTADKSSSTAYMQLSSLTSDDSAVFFCARRDTRHGYYALDYWGQGTSVTVSS |
| 168 | U1 VL | DIVMTQSQKFMSTSVGDRVSVTCKASQNVGTNVAWYQQKPGQSPKALIYSASYRYSGVPDRFTGS |
| | | GSGTDFTLTISNVQSEDLAEYFCQQYNDYPLTFGAGTRLEIK |

TABLE E
Constant Region sequences

| SEQ | | |
|---|---|---|
| ID | Name | Sequence |
| 169 | Murine LC | QPKSSPSVTLFPPSSEELETNKATLVCTITDFYPGVVTVDWKVDGTPVTQGMETTQPS |
| | 1 | KQSNNKYMASSYLTLTARAWERHSSYSCQVTHEGHTVEKSLSRADCS |
| | (Lambda) | |
| 170 | Murine LC | QPKSTPTLTVFPPSSEELKENKATLVCLISNFSPSGVTVAWKANGTPITQGVDTSNPT |
| | 2 | KEGNKFMASSFLHLTSDQWRSHNSFTCQVTHEGDTVEKSLSPAECL |
| | (Lambda) | |
| 171 | Murine LC | (R)ADAAPTVSIFPPSSEQLTSGGASVVCFLNNFYPKDINVKWKIDGSERQNGVLNSW |
| | (Kappa) | TDQDSKDSTYSMSSTLTLTKDEYERHNSYTCEATHKTSTSPIVKSENRNEC |
| 172 | Murine | AKTTPPSVYPLAPGSAAQTNSMVTLGCLVKGYFPEPVTVTWNSGSLSSGVHTFPAVLQ |
| | IgG1 CH1 | SDLYTLSSSVTVPSSPRPSETVTCNVAHPASSTKVDKKI |
| 173 | Murine | VPRDCGCKPCICT |
| | IgG1 | |
| | hinge | |
| 174 | Murine | VPEVSSVFIFPPKPKDVLTITLTPKVTCVVVDISKDDPEVQFSWFVDDVEVHTAQTQP |
| | IgG1 HC | REEQFNSTFRSVSELPIMHQDWLNGKEFKCRVNSAAFPAPIEKTISKTKGRPKAPQVY |
| | Fc (CH2- | TIPPPKEQMAKDKVSLTCMITDFFPEDITVEWQWNGQPAENYKNTQPIMNINGSYFVY |
| | CH3) | SKLNVQKSNWEAGNTFTCSVLHEGLHNHHTEKSLSHSPGK |
| 175 | Murine HC | AKTTAPSVYP LVPVCGGTTG SSVTLGCLVK GYFPEPVTLT WNSGSLSSGV |
| | IgG2a | HTFPALLQSG LYTLSSSVTV TSNTWPSQTI TCNVAHPASS TKVDKKIEPR |
| | | VPITQNPCPP HQRVPPCAAP DLLGGPSVFI FPPKIKDVLM ISLSPMVTCV |
| | | VVDVSEDDPD VQISWFVNNV EVHTAQTQTH REDYNSTLRV VSALPIQHQD |
| | | WMSGKEFKCK VNNRALPSPI EKTISKPRGP VRAPQVYVLP PPAEEMTKKE |
| | | FSLTCMITGF LPAEIAVDWT SNGRTEQNYK NTATVLDSDG SYFMYSKLRV |
| | | QKSTWERGSL FACSVVHEVL HNHLTTKTIS RSLGK |
| 176 | Murine HC | KTTPPSVYPLAPGCGDTTGSSVTLGCLVKGYFPESVTVTWNSGSLSSSVHTFPALLQS |
| | IgG2b | GLYTMSSSVTVPSSTWPSQTVTCSVAHPASSTTVDKKLEPSGPISTINPCPPCKECHK |
| | | CPAPNLEGGPSVFIFPPNIKDVLMISLTPKVTCVVVDVSEDDPDVQISWFVNNVEVHT |
| | | AQTQTHREDYNSTIRVVSTLPIQHQDWMSGKEFKCKVNNKDLPSPIERTISKIKGLVR |
| | | APQVYILPPPAEQLSRKDVSLTCLVVGFNPGDISVEWTSNGHTEENYKDTAPVLDSDG |
| | | SYFIYSKLNMKTSKWEKTDSFSCNVRHEGLKNYYLKKTISRSPGK |
| 177 | Murine HC | TTTAPSVYPLVPGCSDTSGSSVTLGCLVKGYFPEPVTVKWNYGALSSGVRTVSSVLQS |
| | IgG3 | GFYSLSSLVTVPSSTWPSQTVICNVAHPASKTELIKRIEPRIPKPSTPPGSSCPPGNI |
| | | LGGPSVFIFPPKPKDALMISLTPKVTCVVVDVSEDDPDVHVSWFVDNKEVHTAWTQPR |
| | | EAQYNSTFRVVSALPIQHQDWMRGKEFKCKVNNKALPAPIERTISKPKGRAQTPQVYT |
| | | IPPPREQMSKKKVSLTCLVTNFFSEAISVEWERNGELEQDYKNTPPILDSDGTYFLYS |
| | | KLTVDTDSWLQGEIFTCSVVHEALHNHHTQKNLSRSPGK |
| 178 | Human LC | TVAAPSVFI FPPSDEQLKS GTASVVCLLN NFYPREAKVQ WKVDNALQSG |
| | (kappa) | NSQESVTEQD SKDSTYSLSS TLTLSKADYE KHKVYACEVT HQGLSSPVTK |
| | | SFNRGEC |
| 179 | Human LC | RTVAAPSVFI FPPSDEQLKS GTASVVCLLN NFYPREAKVQ WKVDNALQSG |
| | (kappa) | NSQESVTEQD SKDSTYSLSS TLTLSKADYE KHKVYACEVT HQGLSSPVTK |
| | with | SFNRGEC |
| | optional | |
| | junction | |
| | residue | |
| 180 | Human LC | QPKANPTVT LEPPSSEELQ ANKATLVCLI SDFYPGAVTV AWKADGSPVK |
| | (lambda) | AGVETTKPSK QSNNKYAASS YLSLTPEQWK SHRSYSCQVT HEGSTVEKTV |
| | | APTECS |
| 181 | Human LC | GQPKANPTVT LEPPSSEELQ ANKATLVCLI SDFYPGAVTV AWKADGSPVK |
| | (lambda) | AGVETTKPSK QSNNKYAASS YLSLTPEQWK SHRSYSCQVT HEGSTVEKTV |
| | with | APTECS |
| | optional | |
| | junction | |
| | residue | |
| 182 | Human | ASTKGPSVFPLAPSSKSTSGGTAALGCLVKDYFPEPVTVSWNSGALTSGVHTFPAVLQ |
| | IgG1 HC | SSGLYSLSSVVTVPSSSLGTQTYICNVNHKPSNTKVDKKV |
| | CH1 | |
| 183 | Human | EPKSCDKTHTCP |
| | IgG1 HC | |
| | hinge | |
| 184 | Human | PCPAPELLGGPSVFLFPPKPKDTLMISRTPEVTCVVVDVSHEDPEVKENWYVDGVEVH |
| | IgG1 HC | NAKTKPREEQYNSTYRVVSVLTVLHQDWLNGKEYKCKVSNKALPAPIEKTISKAK |
| | Fc (CH2- | GQPREPQVYTLPPSRDELTKNQVSLTCLVKGFYPSDIAVEWESNGQPENNYKTTPPVL |
| | CH3) | DSDGSFFLYSKLTVDKSRWQQGNVFSCSVMHEALHNHYTQKSLSLSPG(K) |
| 185 | Human | ASTKGPSVFPLAPSSKSTSGGTAALGCLVKDYFPEPVTVSWNSGALTSGVHTFPAVLQ |
| | IgG1 HC | SSGLYSLSSVVTVPSSSLGTQTYICNVNHKPSNTKVDKKVEPKSCDKTHTCPPCPAPE |
| | C-domain | LLGGPSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG(K) |
| 186 | Human | ASTKGPSVFPLAPCSRSTSESTAALGCLVKDYFPEPVTVSWNSGALTSGVHTFPAVLQ |
| | IgG4 CH1 | SSGLYSLSSVVTVPSSSLGTKTYTCNVDHKPSNTKVDKRV |
| 187 | Human | ESKYGPPCPSCP |
| | IgG4 | |
| | hinge | |
| 188 | Human | APEFLGGPSVFLFPPKPKDTLMISRTPEVTCVVVDVSQEDPEVQFNWYVDGVEVHNAK |
| | IgG4 Fc | TKPREEQFNSTYRVVSVLTVLHQDWLNGKEYKCKVSNKGLPSSIEKTISKAKGQPREP |
| | (CH2-CH3) | QVYTLPPSQEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLGK |
| 189 | Human | ASTKGPSVFPLAPCSRSTSESTAALGCLVKDYFPEPVTVSWNSGALTSGVHTFPAVLQ |
| | IgG4 HC | SSGLYSLSSVVTVPSSSLGTKTYTCNVDHKPSNTKVDKRVESKYGPPCPSCPAPEFLG |
| | C-domain | GPSVFLFPPKPKDTLMISRTPEVTCVVVDVSQEDPEVQFNWYVDGVEVHNAKTKPREE |
| | | QFNSTYRVVSVLTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLGK |
| 190 | Human | ASTKGPSVFPLAPCSRSTSGGTAALGCLVKDYFPEPVTVSWNSGALTSGVHTFPAVLQ |
| | IgG3 CH1 | SSGLYSLSSVVTVPSSSLGTQTYTCNVNHKPSNTKVDKRV |
| 191 | Human | ELKTPLGDTTHTCPRCPEPKSCDTPPPCPRCPEPKSCDTPPPCPRCPEPKSCDTPPPC |
| | IgG3 | PRCP |
| | hinge | |
| 192 | Human | APELLGGPSVFLFPPKPKDTLMISRTPEVTCVVVDVSHEDPEVQFKWYVDGVEVHNAK |
| | IgG3 Fc | TKPREEQYNSTFRVVSVLTVLHQDWLNGKEYKCKVSNKALPAPIEKTISKTKGQPREP |
| | (CH2-CH3) | QVYTLPPSREEMTKNQVSLTCLVKGFYPSDIAVEWESSGQPENNYNTTPPMLDS |
| | | DGSFFLYSKLTVDKSRWQQGNIFSCSVMHEALHNRFTQKSLSLSPG |
| 193 | Human | ASTKGPSVFP LAPCSRSTSG GTAALGCLVK DYFPEPVTVS WNSGALTSGV |
| | IgG3 HC | HTFPAVLQSS GLYSLSSVVT VPSSSLGTQT YTCNVNHKPS NTKVDKRVEL |
| | C-domain | KTPLGDTTHT CPRCPEPKSC DTPPPCPRCP EPKSCDTPPP CPRCPEPKSC |
| | | DTPPPCPRCP APELLGGPSV FLFPPKPKDT LMISRTPEVT CVVVDVSHED |
| | | PEVQFKWYVD GVEVHNAKTK PREEQYNSTF RVVSVLTVLH QDWLNGKEYK |
| | | CKVSNKALPA PIEKTISKTK GQPREPQVYT LPPSREEMTK NQVSLTCLVK |
| | | GFYPSDIAVE WESSGQPENN YNTTPPMLDS DGSFFLYSKL TVDKSRWQQG |
| | | NIFSCSVMHE ALHNRFTQKS LSLSPGK |
| 194 | Human | ASTKGPSVFPLAPCSRSTSESTAALGCLVKDYFPEPVTVSWNSGALTSGVHTFPAVLQ |
| | IgG2 CH1 | SSGLYSLSSVVTVPSSNFGTQTYTCNVDHKPSNTKVDKTV |
| 195 | Human | ERKCCVECPPCP |
| | IqG2 | |
| | hinge | |
| 196 | Human | APPVAGPSVFLFPPKPKDTLMISRTPEVTCVVVDVSHEDPEVQFNWYVDGVEVHNAKT |
| | IgG2 Fc | KPREEQFNSTFRVVSVLTVVHQDWLNGKEYKCKVSNKGLPAPIEKTISKTKGQPREPQ |
| | (CH2-CH3) | VYTLPPSREEMTKNQVSLTCLVKGFYPSDISVEWESNGQPENNYKTTPPMLDS |
| | | DGSFFLYSKLTVDKSRWQQGNVFSCSVMHEALHNHYTQKSLSLSPGK |
| 197 | Human | ASTKGPSVFP LAPCSRSTSE STAALGCLVK DYFPEPVTVS WNSGALTSGV |
| | IgG2 HC | HTFPAVLQSS GLYSLSSVVT VPSSNFGTQT YTCNVDHKPS NTKVDKTVER |
| | C-domain | KCCVECPPCP APPVAGPSVF LFPPKPKDTL MISRTPEVTC VVVDVSHEDP |
| | | EVQFNWYVDG VEVHNAKTKP REEQFNSTER VVSVLTVVHQ DWLNGKEYKC |
| | | KVSNKGLPAP IEKTISKTKG QPREPQVYTL PPSREEMTKN QVSLTCLVKG |
| | | FYPSDISVEW ESNGQPENNY KTTPPMLDSD GSFFLYSKLT VDKSRWQQGN |
| | | VFSCSVMHEA LHNHYTQKSL SLSPGK |

TABLE F
other sequences

| SEQ | | |
|---|---|---|
| ID | Name | Sequence |
| 198 | Denosumab | EVQLLESGGG LVQPGGSLRL SCAASGFTES SYAMSWVRQA PGKGLEWVSG |
| | heavy | ITGSGGSTYY ADSVKGRFTI SRDNSKNTLY LQMNSLRAED TAVYYCAKDP |
| | chain | GTTVIMSWED PWGQGTLVTV SSASTKGPSV FPLAPCSRST SESTAALGCL |
| | | VKDYFPEPVT VSWNSGALTS GVHTFPAVLQ SSGLYSLSSV VTVPSSNFGT |
| | | QTYTCNVDHK PSNTKVDKTV ERKCCVECPP CPAPPVAGPS VFLFPPKPKD |
| | | TLMISRTPEV TCVVVDVSHE DPEVQFNWYV DGVEVHNAKT KPREEQFNST |
| | | FRVVSVLTVV HQDWLNGKEY KCKVSNKGLP APIEKTISKT KGQPREPQVY |
| | | TLPPSREEMT KNQVSLTCLV KGFYPSDIAV EWESNGQPEN NYKTTPPMLD |
| | | SDGSFFLYSK LTVDKSRWQQ GNVFSCSVMH EALHNHYTQK SLSLSP(GK) |
| | | (Kabat CDRs underlined, VH in bold) |
| 199 | Denosumab | EIVLTQSPGT LSLSPGERAT LSCRASQSVR GRYLAWYQQK PGQAPRLLIY |
| | light | GASSRATGIP DRFSGSGSGT DFTLTISRLE PEDFAVFYCQ QYGSSPRTFG |
| | chain | QGTKVEIKRT VAAPSVFIFP PSDEQLKSGT ASVVCLLNNF YPREAKVQWK |
| | | VDNALQSGNS QESVTEQDSK DSTYSLSSTL TLSKADYEKH KVYACEVTHQ |
| | | GLSSPVTKSF NRGEC |
| | | (Kabat CDRs underlined, VL in bold) |
| 200 | Denosumab | SYAMS |
| | CDR-H1 | |
| 201 | Denosumab | GITGSGGSTYYADSVKG |
| | CDR-H2 | |
| 202 | Denosumab | DPGTTVIMSWEDP |
| | CDR-H3 | |
| 203 | Denosumab | EVQLLESGGG LVQPGGSLRL SCAASGFTFS SYAMSWVRQA PGKGLEWVSG |
| | VH | ITGSGGSTYY ADSVKGRFTI SRDNSKNTLY LQMNSLRAED TAVYYCAKDP |
| | | GTTVIMSWED PWGQGTLVTV SS |
| 204 | Denosumab | ASTKGPSVFPLAPCSRSTSESTAALGCLVKDYFPEPVTVSWNSGALTSGVHTFPAVLQ |
| | CH | SSGLYSLSSVVTVPSSNFGTQTYTCNVDHKPSNTKVDKTVERKCCVECPPCPAPPVAG |
| | | PSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPP |
| | | SREEMTKNQVSLTCLVKGFYPSDIAVEWESNGQPENNYKTTPPMLDSDGSFFLYSKLT |
| | | VDKSRWQQGNVFSCSVMHEALHNHYTQKSLSLSPGK |
| 205 | Denosumab | RASQSVRGRYLA |
| | CDR-L1 | |
| 206 | Denosumab | GASSRAT |
| | CDR-L2 | |
| 207 | Denosumab | QQYGSSPRT |
| | CDR-L3 | |
| 208 | Denosumab | EIVLTQSPGT LSLSPGERAT LSCRASQSVR GRYLAWYQQK PGQAPRLLIY |
| | VL | GASSRATGIP DRFSGSGSGT DFTLTISRLE PEDFAVFYCQ QYGSSPRTFG |
| | | QGTKVEIKRT V |
| 209 | Denosumab | AAPSVFIFPPSDEQLKSGTASVVCLLNNFYPREAKVQWKVDNALQSGNSQESVTEQDS |
| | CL | KDSTYSLSSTLTLSKADYEKHKVYACEVTHQGLSSPVTKSFNRGEC |
| 210 | Human OPG | ETFPPKYLHYDEETSHQLLCDKCPPGTYLKQHCTAKWKTVCAPCPDHYYTDSWHTSDE |
| | | CLYCSPVCKELQYVKQECNRTHNRVCECKEGRYLEIEFCLKHRSCPPGFGVVQAGTPE |
| | | RNTVCKRCPDGFFSNETSSKAPCRKHTNCSVFGLLLTQKGNATHDNICSGNSESTQKC |
| | | GIDVTLCEEAFFRFAVPTKFTPNWLSVLVDNLPGTKVNAESVERIKRQHSSQEQTFQL |
| | | LKLWKHQNKDQDIVKKIIQDIDLCENSVQRHIGHANLTFEQLRSLMESLPGKKVGAED |
| | | IEKTIKACKPSDQILKLLSLWRIKNGDQDTLKGLMHALKHSKTYHFPKTVTQSLKKTI |
| | | RFLHSFTMYKLYQKLFLEMIGNQVQSVKISCL |
| 211 | Human OPG | MNNLLCCALVELDISIKWTTQETFPPKYLHYDEETSHQLLCDKCPPGTYLKQHCTAKW |
| | with | KTVCAPCPDHYYTDSWHTSDECLYCSPVCKELQYVKQECNRTHNRVCECKEGRYLEIE |
| | signal | FCLKHRSCPPGFGVVQAGTPERNTVCKRCPDGFFSNETSSKAPCRKHTNCSVFGLLLT |
| | peptide | QKGNATHDNICSGNSESTQKCGIDVTLCEEAFFRFAVPTKFTPNWLSVLVDNLPGTKV |
| | | NAESVERIKRQHSSQEQTFQLLKLWKHQNKDQDIVKKIIQDIDLCENSVQRHIGHANL |
| | | TFEQLRSLMESLPGKKVGAEDIEKTIKACKPSDQILKLLSLWRIKNGDQDTLKGLMHA |
| | | LKHSKTYHFPKTVTQSLKKTIRFLHSFTMYKLYQKLFLEMIGNQVQSVKISCL |
| 212 | Human | MRRASRDYTKYLRGSEEMGGGPGAPHEGPLHAPPPPAPHQPPAASRSMFVALLGLGLG |
| | RANKL | QVVCSVALFFYFRAQMDPNRISEDGTHCIYRILRLHENADFQDTTLESQDTKLIPDSC |
| | (membrane | RRIKQAFQGAVQKELQHIVGSQHIRAEKAMVDGSWLDLAKRSKLEAQPFAHLTINATD |
| | bound) | IPSGSHKVSLSSWYHDRGWAKISNMTFSNGKLIVNQDGFYYLYANICFRHHETSGDLA |
| | | TEYLQLMVYVTKTSIKIPSSHTLMKGGSTKYWSGNSEFHFYSINVGGFFKLRSGEEIS |
| | | IEVSNPSLLDPDQDATYFGAFKVRDID |
| 213 | RANKL | FYFRAQMDPNRISEDGTHCIYRILRLHENADFQDTTLESQDTKLIPDSCRRIKQAFQG |
| | (soluble | AVQKELQHIVGSQHIRAEKAMVDGSWLDLAKRSKLEAQPFAHLTINATDIPSGSHKVS |
| | form) | LSSWYHDRGWAKISNMTFSNGKLIVNQDGFYYLYANICFRHHETSGDLATEYLQLMVY |
| | | VTKTSIKIPSSHTLMKGGSTKYWSGNSEFHFYSINVGGFFKLRSGEEISIEVSNPSLL |
| | | DPDQDATYFGAFKVRDID |
| 214 | RANKL | IRAEKAMVDGSWLDLAKRSKLEAQPFAHLTINATDIPSGSHKVSLSSWYHDRGWAKIS |
| | (soluble, | NMTFSNGKLIVNQDGFYYLYANICFRHHETSGDLATEYLQLMVYVTKTSIKIPSSHTL |
| | via | MKGGSTKYWSGNSEFHFYSINVGGFFKLRSGEEISIEVSNPSLLDPDQDATYFGAFKV |
| | proteolytic | RDID |
| | cleavage) | |
| 215 | RANKL | DLATE |
| | epitope | |
| 216 | RANKL DE | GFYYLYANICFRHHETSGDLATEYLQLMVYVTKTSIKIP |
| | loop | |

1. A method of detecting an anti-drug antibody (ADA) in a sample, wherein said ADA binds to denosumab, comprising mixing said sample with:
(a) a first antibody or antibody conjugate that binds to RANKL, comprising: the heavy chain (HC) CDR-H1, CDR-H2, and CDR-H3 of SEQ ID NO:203, and the light chain (LC) CDR-L1, CDR-L2, and CDR-L3 of SEQ ID NO:208; wherein said first antibody or antibody conjugate can be attached to a solid substrate;
(b) (i) a second antibody that binds to RANKL, wherein said second antibody comprises an amino acid sequence that differs from the first antibody by at least one amino acid in one of the six CDR sequences; or (ii) an osteoprotegerin (OPG) protein that binds to RANKL; and
(c) a third antibody that binds to said ADA and can be linked to a detectable marker.

2. The method of claim 1, wherein said first antibody conjugate comprises an antibody molecule noncovalently or covalently linked to an immobilizing moiety, wherein said solid substrate comprises a binding partner, and said immobilizing moiety can be attached to a solid substrate by binding to said binding partner.

3. The method of claim 1, wherein said first antibody or antibody conjugate comprise a CDR-H1 comprising SEQ ID NO:200, a CDR-H2 comprising SEQ ID NO:201, and a CDR-H3 comprising SEQ ID NO:202; and a CDR-L1 comprising SEQ ID NO:205, a CDR-L2 comprising SEQ ID NO:206, and a CDR-L3 comprising SEQ ID NO:207.

4. The method of claim 1, wherein said first antibody or antibody conjugate comprises a heavy chain variable region (VH) that is at least 90% identical to SEQ ID NO:203, and a light chain variable region (VL) that is at least 90% identical to SEQ ID NO:208.

5. The method of claim 1, wherein said first antibody or antibody conjugate comprises a human heavy chain constant domain (CH) or a murine heavy chain constant domain (CH).

6. The method of claim 1, wherein said first antibody or antibody conjugate comprises a human Fc domain or a murine Fc domain.

7. The method of claim 1, wherein said first antibody or antibody conjugate comprises a human light chain constant (CL) domain or a murine light chain constant (CL) domain.

8. The method of claim 1, wherein said third antibody is covalently or noncovalently linked to a detectable marker.

9. The method of claim 1, said detectable marker is an isotope, an enzyme, a fluorescent moiety, a luminescent moiety, a chromatic moiety, a metal, or an electric charge.

10. The method of claim 1, wherein said third antibody comprises six CDR sequences that are identical to that of the first antibody.

11. The method of claim 1, wherein said third antibody comprises a VH domain comprising the same sequence as the VH of the first antibody, and a VL domain comprising the same sequence as the VL domain of the first antibody.

12. The method of claim 1, wherein said third antibody comprises a heavy chain (HC) comprising the same sequence as the HC of the first antibody, and a light chain (LC) comprising the same sequence as the LC of the first antibody.

13. The method of claim 1, wherein said second antibody binds to the same epitope as denosumab.

14. The method of claim 1, wherein said second antibody competes with denosumab for RANKL binding.

15. The method of claim 1, wherein said second antibody comprises an amino acid sequence that differs from the first antibody by at least one amino acid in each of the six CDR sequences.

16. The method of claim 1, wherein said second antibody comprises: (i) the heavy chain CDR-H1, CDR-H2, and CDR3-H3 of SEQ ID NO: 127, 129, 131, 133, 135, 137, 139, 141, 143, 145, 147, 149, 151, 153, 155, 157, 159, 161, 163, 165, or 167; and (ii) the light chain CDR-L1, CDR-L2, and CDR3 of SEQ ID NO: 128, 130, 132, 134, 136, 138, 140, 142, 144, 146, 148, 150, 152, 154, 156, 158, 160, 162, 164, 166, or 168.

17. The method of claim 1, wherein said second antibody comprises a human heavy chain constant domain (CH) or a murine heavy chain constant domain (CH).

18. The method of claim 1, wherein said second antibody comprises a human Fc domain or a murine Fc domain.

19. The method of claim 1, wherein said second antibody comprises a human CL domain or a murine CL domain.

20. A diagnostic kit for detecting an anti-drug antibody (ADA) in a sample, wherein said ADA binds to denosumab, comprising:
(a) a first antibody or antibody conjugate that binds to RANKL, comprising: the heavy chain (HC) CDR-H1, CDR-H2, and CDR-H3 of SEQ ID NO:203, and the light chain (LC) CDR-L1, CDR-L2, and CDR-L3 of SEQ ID NO:208; wherein said first antibody or antibody conjugate can be attached to a solid substrate;
(b) (i) a second antibody that binds to RANKL, wherein said second antibody comprises an amino acid sequence that differs from the first antibody by at least one amino acid in one of the six CDR sequences; or (ii) an osteoprotegerin (OPG) protein that binds to RANKL; and
(c) a third antibody that binds to said ADA and can be linked to a detectable marker; and
(d) instructions for use.

21. An anti-RANKL antibody, or antigen binding fragment thereof, comprising a heavy chain variable domain (VH) and a light chain variable domain (VL), wherein said VH and VL comprise (i) the heavy chain CDR-H1, CDR-H2, and CDR3-H3 of SEQ ID NO: 139; and (ii) the light chain CDR-L1, CDR-L2, and CDR3 of SEQ ID NO: 140, wherein the CDRs are defined according to Kabat, Chothia, IMGT, AbM, North, Martin, or Contact.

22. The anti-RANKL antibody, or antigen binding fragment thereof, of claim 21, comprising a CDR-H1, a CDR-H2, a CDR-H3, a CDR-L1, a CDR-L2, and a CDR-L3 comprising SEQ ID NOs. 37-42, respectively.


SEQUENCE LISTING (216 sequences)

SEQ ID NO: 1 - protein, 5 aa, synthetic construct
  REGION 1..5: note = Synthetic Polypeptide
SNGMH

SEQ ID NO: 2 - protein, 16 aa, synthetic construct
  REGION 1..16: note = Synthetic Polypeptide
GIGTAGGTYYADSVKG

SEQ ID NO: 3 - protein, 8 aa, synthetic construct
  REGION 1..8: note = Synthetic Polypeptide
KNWGWFDP

SEQ ID NO: 4 - protein, 11 aa, synthetic construct
  REGION 1..11: note = Synthetic Polypeptide
RASQSVNSYLA

SEQ ID NO: 5 - protein, 7 aa, synthetic construct
  REGION 1..7: note = Synthetic Polypeptide
DASNRAT

SEQ ID NO: 6 - protein, 10 aa, synthetic construct
  REGION 1..10: note = Synthetic Polypeptide
QQRSNWPPFT

SEQ ID NO: 7 - protein, 5 aa, synthetic construct
  REGION 1..5: note = Synthetic Polypeptide
SNGMH

SEQ ID NO: 8 - protein, 16 aa, synthetic construct
  REGION 1..16: note = Synthetic Polypeptide
GIGTAGGTYYADSVKG

SEQ ID NO: 9 - protein, 8 aa, synthetic construct
  REGION 1..8: note = Synthetic Polypeptide
KDWGWFDP

SEQ ID NO: 10 - protein, 11 aa, synthetic construct
  REGION 1..11: note = Synthetic Polypeptide
RASQSISSYLA

SEQ ID NO: 11 - protein, 7 aa, synthetic construct
  REGION 1..7: note = Synthetic Polypeptide
DASNRAT

SEQ ID NO: 12 - protein, 10 aa, synthetic construct
  REGION 1..10: note = Synthetic Polypeptide
QQRSKWPPYT

SEQ ID NO: 13 - protein, 5 aa, synthetic construct
  REGION 1..5: note = Synthetic Polypeptide
SYGMH

SEQ ID NO: 14 - protein, 16 aa, synthetic construct
  REGION 1..16: note = Synthetic Polypeptide
GIGTGGGTYYADSVKG

SEQ ID NO: 15 - protein, 8 aa, synthetic construct
  REGION 1..8: note = Synthetic Polypeptide
KNWGWFDY

SEQ ID NO: 16 - protein, 11 aa, synthetic construct
  REGION 1..11: note = Synthetic Polypeptide
RASQSISSYLA

SEQ ID NO: 17 - protein, 7 aa, synthetic construct
  REGION 1..7: note = Synthetic Polypeptide
DASNRAT

SEQ ID NO: 18 - protein, 10 aa, synthetic construct
  REGION 1..10: note = Synthetic Polypeptide
QQRSKWPPYT

SEQ ID NO: 19 - protein, 5 aa, synthetic construct
  REGION 1..5: note = Synthetic Polypeptide
GHALH

SEQ ID NO: 20 - protein, 16 aa, synthetic construct
  REGION 1..16: note = Synthetic Polypeptide
GIGTHGGTYYADSVKG

SEQ ID NO: 21 - protein, 8 aa, synthetic construct
  REGION 1..8: note = Synthetic Polypeptide
RNWGQFDY

SEQ ID NO: 22 - protein, 11 aa, synthetic construct
  REGION 1..11: note = Synthetic Polypeptide
RASQSVSSYLA

SEQ ID NO: 23 - protein, 7 aa, synthetic construct
  REGION 1..7: note = Synthetic Polypeptide
DASNRAT

SEQ ID NO: 24 - protein, 10 aa, synthetic construct
  REGION 1..10: note = Synthetic Polypeptide
QQRSNWPPYT

SEQ ID NO: 25 - protein, 5 aa, synthetic construct
  REGION 1..5: note = Synthetic Polypeptide
AYPMH

SEQ ID NO: 26 - protein, 16 aa, synthetic construct
  REGION 1..16: note = Synthetic Polypeptide
GIGSGGGTNYADSVKG

SEQ ID NO: 27 - protein, 7 aa, synthetic construct
  REGION 1..7: note = Synthetic Polypeptide
GRNSFDY

SEQ ID NO: 28 - protein, 11 aa, synthetic construct
  REGION 1..11: note = Synthetic Polypeptide
RASQGISTWLA

SEQ ID NO: 29 - protein, 7 aa, synthetic construct
  REGION 1..7: note = Synthetic Polypeptide
AASSLQS

SEQ ID NO: 30 - protein, 9 aa, synthetic construct
  REGION 1..9: note = Synthetic Polypeptide
QQYNSYPPT

SEQ ID NO: 31 - protein, 5 aa, synthetic construct
  REGION 1..5: note = Synthetic Polypeptide
HYPLH

SEQ ID NO: 32 - protein, 16 aa, synthetic construct
  REGION 1..16: note = Synthetic Polypeptide
GIHTGGGTYYTDSVKG

SEQ ID NO: 33 - protein, 7 aa, synthetic construct
  REGION 1..7: note = Synthetic Polypeptide
GRNSFDY

SEQ ID NO: 34 - protein, 11 aa, synthetic construct
  REGION 1..11: note = Synthetic Polypeptide
RASQGISSWLA

SEQ ID NO: 35 - protein, 7 aa, synthetic construct
  REGION 1..7: note = Synthetic Polypeptide
AASSLQS

SEQ ID NO: 36 - protein, 9 aa, synthetic construct
  REGION 1..9: note = Synthetic Polypeptide
QQYNSYPPT

SEQ ID NO: 37 - protein, 5 aa, synthetic construct
  REGION 1..5: note = Synthetic Polypeptide
RYAMN

SEQ ID NO: 38 - protein, 17 aa, synthetic construct
  REGION 1..17: note = Synthetic Polypeptide
GISTSGGSTYYADSVKG

SEQ ID NO: 39 - protein, 13 aa, synthetic construct
  REGION 1..13: note = Synthetic Polypeptide
DGDRGVIMTVFDY

SEQ ID NO: 40 - protein, 12 aa, synthetic construct
  REGION 1..12: note = Synthetic Polypeptide
RASQSVRSSYLA

SEQ ID NO: 41 - protein, 7 aa, synthetic construct
  REGION 1..7: note = Synthetic Polypeptide
GASSRAT

SEQ ID NO: 42 - protein, 9 aa, synthetic construct
  REGION 1..9: note = Synthetic Polypeptide
QQYGSSPRT

SEQ ID NO: 43 - protein, 5 aa, synthetic construct
  REGION 1..5: note = Synthetic Polypeptide
NYAIH

SEQ ID NO: 44 - protein, 17 aa, synthetic construct
  REGION 1..17: note = Synthetic Polypeptide
WINAGNGNTKFSQKFQG

SEQ ID NO: 45 - protein, 17 aa, synthetic construct
  REGION 1..17: note = Synthetic Polypeptide
DSSNMVRGIIIAYYFDY

SEQ ID NO: 46 - protein, 11 aa, synthetic construct
  REGION 1..11: note = Synthetic Polypeptide
RASQSISRYLN

SEQ ID NO: 47 - protein, 7 aa, synthetic construct
  REGION 1..7: note = Synthetic Polypeptide
GASSLQS

SEQ ID NO: 48 - protein, 5 aa, synthetic construct
  REGION 1..5: note = Synthetic Polypeptide
QHTRA

SEQ ID NO: 49 - protein, 5 aa, synthetic construct
  REGION 1..5: note = Synthetic Polypeptide
NYAIH

SEQ ID NO: 50 - protein, 17 aa, synthetic construct
  REGION 1..17: note = Synthetic Polypeptide
WINAGNGNTKFSQKFQG

SEQ ID NO: 51 - protein, 17 aa, synthetic construct
  REGION 1..17: note = Synthetic Polypeptide
DSSNMVRGIIIAYYFDY

SEQ ID NO: 52 - protein, 11 aa, synthetic construct
  REGION 1..11: note = Synthetic Polypeptide
RASQSVGSYLA

SEQ ID NO: 53 - protein, 7 aa, synthetic construct
  REGION 1..7: note = Synthetic Polypeptide
DATNRAT

SEQ ID NO: 54 - protein, 5 aa, synthetic construct
  REGION 1..5: note = Synthetic Polypeptide
QHRRT

SEQ ID NO: 55 - protein, 5 aa, synthetic construct
  REGION 1..5: note = Synthetic Polypeptide
DYAMH

SEQ ID NO: 56 - protein, 17 aa, synthetic construct
  REGION 1..17: note = Synthetic Polypeptide
GISWNSGRIGYADSVKG

SEQ ID NO: 57 - protein, 14 aa, synthetic construct
  REGION 1..14: note = Synthetic Polypeptide
GGSTSARYSSGWYY

SEQ ID NO: 58 - protein, 11 aa, synthetic construct
  REGION 1..11: note = Synthetic Polypeptide
SGDALPKQYVY

SEQ ID NO: 59 - protein, 7 aa, synthetic construct
  REGION 1..7: note = Synthetic Polypeptide
EDSERPS

SEQ ID NO: 60 - protein, 11 aa, synthetic construct
  REGION 1..11: note = Synthetic Polypeptide
QSTDSSGTYVV

SEQ ID NO: 61 - protein, 5 aa, synthetic construct
  REGION 1..5: note = Synthetic Polypeptide
NYPMH

SEQ ID NO: 62 - protein, 17 aa, synthetic construct
  REGION 1..17: note = Synthetic Polypeptide
VISYDGNNKYYADSVKG

SEQ ID NO: 63 - protein, 7 aa, synthetic construct
  REGION 1..7: note = Synthetic Polypeptide
GGGGFDY

SEQ ID NO: 64 - protein, 12 aa, synthetic construct
  REGION 1..12: note = Synthetic Polypeptide
RASQSVSSSSLA

SEQ ID NO: 65 - protein, 7 aa, synthetic construct
  REGION 1..7: note = Synthetic Polypeptide
GASSRAT

SEQ ID NO: 66 - protein, 5 aa, synthetic construct
  REGION 1..5: note = Synthetic Polypeptide
QQYGA

SEQ ID NO: 67 - protein, 5 aa, synthetic construct
  REGION 1..5: note = Synthetic Polypeptide
NYWMS

SEQ ID NO: 68 - protein, 17 aa, synthetic construct
  REGION 1..17: note = Synthetic Polypeptide
SISSSSSYIYYADSVKG

SEQ ID NO: 69 - protein, 11 aa, synthetic construct
  REGION 1..11: note = Synthetic Polypeptide
QSESSVYGMDV

SEQ ID NO: 70 - protein, 13 aa, synthetic construct
  REGION 1..13: note = Synthetic Polypeptide
SGSHSNIASSSAN

SEQ ID NO: 71 - protein, 7 aa, synthetic construct
  REGION 1..7: note = Synthetic Polypeptide
GNDQRPS

SEQ ID NO: 72 - protein, 11 aa, synthetic construct
  REGION 1..11: note = Synthetic Polypeptide
AAWDDSLSGVV

SEQ ID NO: 73 - protein, 5 aa, synthetic construct
  REGION 1..5: note = Synthetic Polypeptide
NAWMS

SEQ ID NO: 74 - protein, 17 aa, synthetic construct
  REGION 1..17: note = Synthetic Polypeptide
SISSSSSYIYYADSVKG

SEQ ID NO: 75 - protein, 13 aa, synthetic construct
  REGION 1..13: note = Synthetic Polypeptide
DGSIFGVVTPFDY

SEQ ID NO: 76 - protein, 14 aa, synthetic construct
  REGION 1..14: note = Synthetic Polypeptide
TGSSSNIGAGYDVH

SEQ ID NO: 77 - protein, 7 aa, synthetic construct
  REGION 1..7: note = Synthetic Polypeptide
SNNQRPS

SEQ ID NO: 78 - protein, 13 aa, synthetic construct
  REGION 1..13: note = Synthetic Polypeptide
AAWDDSLRGPHWV

SEQ ID NO: 79 - protein, 5 aa, synthetic construct
  REGION 1..5: note = Synthetic Polypeptide
NTWMN

SEQ ID NO: 80 - protein, 17 aa, synthetic construct
  REGION 1..17: note = Synthetic Polypeptide
SISVNSNYIYYADSVKG

SEQ ID NO: 81 - protein, 8 aa, synthetic construct
  REGION 1..8: note = Synthetic Polypeptide
DSEYAFQN

SEQ ID NO: 82 - protein, 14 aa, synthetic construct
  REGION 1..14: note = Synthetic Polypeptide
TGSSSNIGAGYDVH

SEQ ID NO: 83 - protein, 7 aa, synthetic construct
  REGION 1..7: note = Synthetic Polypeptide
DNEWRPS

SEQ ID NO: 84 - protein, 10 aa, synthetic construct
  REGION 1..10: note = Synthetic Polypeptide
ATWDDSLHWV

SEQ ID NO: 85 - protein, 5 aa, synthetic construct
  REGION 1..5: note = Synthetic Polypeptide
DHWMS

SEQ ID NO: 86 - protein, 17 aa, synthetic construct
  REGION 1..17: note = Synthetic Polypeptide
SISSSSSYIYYADSVKG

SEQ ID NO: 87 - protein, 12 aa, synthetic construct
  REGION 1..12: note = Synthetic Polypeptide
HEDTYYYYGMDV

SEQ ID NO: 88 - protein, 13 aa, synthetic construct
  REGION 1..13: note = Synthetic Polypeptide
SGSFSNIGGNTVN

SEQ ID NO: 89 - protein, 7 aa, synthetic construct
  REGION 1..7: note = Synthetic Polypeptide
DNDQRPS

SEQ ID NO: 90 - protein, 11 aa, synthetic construct
  REGION 1..11: note = Synthetic Polypeptide
ASWDDSLSGVV

SEQ ID NO: 91 - protein, 5 aa, synthetic construct
  REGION 1..5: note = Synthetic Polypeptide
KDWMS

SEQ ID NO: 92 - protein, 17 aa, synthetic construct
  REGION 1..17: note = Synthetic Polypeptide
VISYDGSNKYYADSVKG

SEQ ID NO: 93 - protein, 11 aa, synthetic construct
  REGION 1..11: note = Synthetic Polypeptide
AAPHYYYGLDV

SEQ ID NO: 94 - protein, 13 aa, synthetic construct
  REGION 1..13: note = Synthetic Polypeptide
SGSSSNIGSNTVN

SEQ ID NO: 95 - protein, 7 aa, synthetic construct
  REGION 1..7: note = Synthetic Polypeptide
YDDLLPS

SEQ ID NO: 96 - protein, 11 aa, synthetic construct
  REGION 1..11: note = Synthetic Polypeptide
GSWDSSLSAVV

SEQ ID NO: 97 - protein, 5 aa, synthetic construct
  REGION 1..5: note = Synthetic Polypeptide
GFGMH

SEQ ID NO: 98 - protein, 17 aa, synthetic construct
  REGION 1..17: note = Synthetic Polypeptide
VISYDGRNKYYADSVKG

SEQ ID NO: 99 - protein, 13 aa, synthetic construct
  REGION 1..13: note = Synthetic Polypeptide
SLSGTYWGYGMDV

SEQ ID NO: 100 - protein, 11 aa, synthetic construct
  REGION 1..11: note = Synthetic Polypeptide
RASQSIRSWLA

SEQ ID NO: 101 - protein, 7 aa, synthetic construct
  REGION 1..7: note = Synthetic Polypeptide
KASSLES

SEQ ID NO: 102 - protein, 9 aa, synthetic construct
  REGION 1..9: note = Synthetic Polypeptide
QQYNSYLWT

SEQ ID NO: 103 - protein, 5 aa, synthetic construct
  REGION 1..5: note = Synthetic Polypeptide
GYGIH

SEQ ID NO: 104 - protein, 17 aa, synthetic construct
  REGION 1..17: note = Synthetic Polypeptide
VISYDGTNKYYGDSVKG

SEQ ID NO: 105 - protein, 13 aa, synthetic construct
  REGION 1..13: note = Synthetic Polypeptide
SLSGTYWGYGMDV

SEQ ID NO: 106 - protein, 11 aa, synthetic construct
  REGION 1..11: note = Synthetic Polypeptide
RASQNIGNWLA

SEQ ID NO: 107 - protein, 7 aa, synthetic construct
  REGION 1..7: note = Synthetic Polypeptide
KASSLQN

SEQ ID NO: 108 - protein, 9 aa, synthetic construct
  REGION 1..9: note = Synthetic Polypeptide
QRYNSYSWT

SEQ ID NO: 109 - protein, 5 aa, synthetic construct
  REGION 1..5: note = Synthetic Polypeptide
GYGMH

SEQ ID NO: 110 - protein, 17 aa, synthetic construct
  REGION 1..17: note = Synthetic Polypeptide
VISYDGSNKHYADSVKG

SEQ ID NO: 111 - protein, 13 aa, synthetic construct
  REGION 1..13: note = Synthetic Polypeptide
SLSGTYWGYGMDV

SEQ ID NO: 112 - protein, 11 aa, synthetic construct
  REGION 1..11: note = Synthetic Polypeptide
RASQNISSWLA

SEQ ID NO: 113 - protein, 7 aa, synthetic construct
  REGION 1..7: note = Synthetic Polypeptide
KASSLES

SEQ ID NO: 114 - protein, 9 aa, synthetic construct
  REGION 1..9: note = Synthetic Polypeptide
QQYNRYSWT

SEQ ID NO: 115 - protein, 5 aa, synthetic construct
  REGION 1..5: note = Synthetic Polypeptide
NYYIE

SEQ ID NO: 116 - protein, 17 aa, synthetic construct
  REGION 1..17: note = Synthetic Polypeptide
VINPGWGDTNYNEKFKG

SEQ ID NO: 117 - protein, 12 aa, synthetic construct
  REGION 1..12: note = Synthetic Polypeptide
RDTAHGYYALDP

SEQ ID NO: 118 - protein, 30 aa, synthetic construct
  REGION 1..30: note = Synthetic Polypeptide
LYSALASERASNVALGLYTHRASNVALALA

SEQ ID NO: 119 - protein, 7 aa, synthetic construct
  REGION 1..7: note = Synthetic Polypeptide
SASYRYS

SEQ ID NO: 120 - protein, 9 aa, synthetic construct
  REGION 1..9: note = Synthetic Polypeptide
QQYWDYPLT

SEQ ID NO: 121 - protein, 5 aa, synthetic construct
  REGION 1..5: note = Synthetic Polypeptide
NYYIE

SEQ ID NO: 122 - protein, 17 aa, synthetic construct
  REGION 1..17: note = Synthetic Polypeptide
VINPGWGDTNYNEKFKG

SEQ ID NO: 123 - protein, 12 aa, synthetic construct
  REGION 1..12: note = Synthetic Polypeptide
RDTRHGYYALDY

SEQ ID NO: 124 - protein, 30 aa, synthetic construct; identical to SEQ ID NO: 118
  REGION 1..30: note = Synthetic Polypeptide

SEQ ID NO: 125 - protein, 7 aa, synthetic construct
  REGION 1..7: note = Synthetic Polypeptide
SASYRYS

SEQ ID NO: 126 - protein, 9 aa, synthetic construct
  REGION 1..9: note = Synthetic Polypeptide
QQYNDYPLT

SEQ ID NO: 127 - protein, 116 aa, synthetic construct
  REGION 1..116: note = Synthetic Polypeptide
EVQLVQSGGGLVHPGGSLRLSCEGSGFTFSSNGMHWVRQTPGKGLEWVSGIGTAGGTYYADSVKGRFTISRDNVKKSLYLQMNSLRAEDMAIYYCVRKNWGWFDPWGQGALVTVSS

SEQ ID NO: 128 - protein, 109 aa, synthetic construct
  REGION 1..109: note = Synthetic Polypeptide
EIVLTQSPATLSLSPGERATLSCRASQSVNSYLAWFQQKPGQAPRLLIYDASNRATGIPARFSGSGSGTDFTLTISSLEPEDFAIYYCQQRSNWPPFTFGQGTKLEIKR

SEQ ID NO: 129 - protein, 116 aa, synthetic construct
  REGION 1..116: note = Synthetic Polypeptide
EVQLVQSGGGLVHPGGSLRLSCEGSGFTFSSNGMHWVRQTPGKGLEWVSGIGTAGGTYYADSVKGRFTISRDNVKKSLYLQMNSLRAEDMAIYYCVRKDWGWFDPWGQGALVTVSS

SEQ ID NO: 130 - protein, 109 aa, synthetic construct
  REGION 1..109: note = Synthetic Polypeptide
EIVLTQSPATLSLSPGERATLSCRASQSISSYLAWYQQKPGQAPRLLIYDASNRATGIPARFSGSGSGTDFTLTISSLEPEDFAVYYCQQRSKWPPYTFGQGTKLEIKR

SEQ ID NO: 131 - protein, 116 aa, synthetic construct
  REGION 1..116: note = Synthetic Polypeptide
EVQLVQSGGGLVHPGGSLRLSCAGSGFTFSSYGMHWVRQAPGKGLEWVSGIGTGGGTYYADSVKGRFTISRDNVKNSLYLQMNSLRAEDMAVYYCARKNWGWFDYWGQGTLVTVSS

SEQ ID NO: 132 - protein, 109 aa, synthetic construct; identical to SEQ ID NO: 130
  REGION 1..109: note = Synthetic Polypeptide

SEQ ID NO: 133 - protein, 116 aa, synthetic construct
  REGION 1..116: note = Synthetic Polypeptide
EVQLVQSGGGLVHPGGSLRLSCAGSGFTFSGHALHWVRQAPGKGLEWVSGIGTHGGTYYADSVKGRFTISRDNAKNSLFLQMNSLSAEDMAVYYCTRRNWGQFDYWGQGTLVTVSS

SEQ ID NO: 134 - protein, 109 aa, synthetic construct
  REGION 1..109: note = Synthetic Polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPYTFGQGTKLEIKR

SEQ ID NO: 135 - protein, 115 aa, synthetic construct
  REGION 1..115: note = Synthetic Polypeptide
EVQLVQSGGGLVHPGGSLRLSCVGSRFTFSAYPMHWVRQAPGKGLEWVSGIGSGGGTNYADSVKGRFTISRDTAKNSLYLQMNSLRAEDMAVYYCARGRNSFDYWGQGTLVTVSS

SEQ ID NO: 136 - protein, 108 aa, synthetic construct
  REGION 1..108: note = Synthetic Polypeptide
DIQMTQSPSSLSASVGDRVTITCRASQGISTWLAWYQQKPEKAPKSLIYAASSLQSGVPSRFSGSGSGTDFTLTISSLQPEDFATYYCQQYNSYPPTFGQGTKVEIKR

SEQ ID NO: 137 - protein, 115 aa, synthetic construct
  REGION 1..115: note = Synthetic Polypeptide
EVQLVQSGGDLVHPGGSLRLSCVGSGFTFSHYPLHWVRQAPGKGLEWISGIHTGGGTYYTDSVKGRFTISSDNAKNSLYLQMNTLRAEDMAVYYCARGRNSFDYWGQGTLVIVSS

SEQ ID NO: 138 - protein, 108 aa, synthetic construct
  REGION 1..108: note = Synthetic Polypeptide
DIQMTQSPSSLSASVGDRVTITCRASQGISSWLAWYQQKPEKAPKSLIYAASSLQSGVPSRFSGSGSGTDFTLTISSLQSEDFATYYCQQYNSYPPTFGQGTRLEIKR

SEQ ID NO: 139 - protein, 122 aa, synthetic construct
  REGION 1..122: note = Synthetic Polypeptide
EVHLLESGGGLVQPGGSLRLSCAASGFTFSRYAMNWVRQAPGKGLEWVSGISTSGGSTYYADSVKGRFTISRDISRNTLNLQMNSLRAEDTAVYYCAKDGDRGVIMTVFDYWGQGTLVTVSS

SEQ ID NO: 140 - protein, 109 aa, synthetic construct
  REGION 1..109: note = Synthetic Polypeptide
EIVLTQSPGTLSLSPGERATLSCRASQSVRSSYLAWYQQKPGQAPRLLIYGASSRATGIPDRFSGSGSGTDFTLTISRLEPEDFAVYYCQQYGSSPRTFGQGTKVEIKR

SEQ ID NO: 141 - protein, 126 aa, synthetic construct
  REGION 1..126: note = Synthetic Polypeptide
QVQLVQSGAEVRKPGASVKVSCKASGYDFSNYAIHWVRQAPGQRLEWMGWINAGNGNTKFSQKFQGRITVTRDTAASTAYMELRSLRSEDTAVYYCARDSSNMVRGIIIAYYFDYWGQGTLVTVSS

SEQ ID NO: 142 - protein, 104 aa, synthetic construct
  REGION 1..104: note = Synthetic Polypeptide
EIVMTQSPSSLSASVGDRVTITCRASQSISRYLNWYQLKPGKAPRLLIYGASSLQSGVPSRFSGSGSGAEFTLTISSLQPEDIATYYCQHTRAFGQGTKVEIKR

SEQ ID NO: 143 - protein, 126 aa, synthetic construct
  REGION 1..126: note = Synthetic Polypeptide
EVQLVQSGAEVRKPGASVKVSCKASGYDFSNYAIHWVRQAPGQRLEWMGWINAGNGNTKFSQKFQGRITVTRDTAASTAYMELRSLRSEDTAVYYCARDSSNMVRGIIIAYYFDYWGQGTLVTVSS

SEQ ID NO: 144 - protein, 104 aa, synthetic construct
  REGION 1..104: note = Synthetic Polypeptide
EIVLTQSPATLSFSPGERATLSCRASQSVGSYLAWYQQRPGQAPRPLIYDATNRATGIPTRFSGSGSGTDFTLTISSLEPEDFATYYCQHRRTFGRGTKLEIKR

SEQ ID NO: 145 - protein, 123 aa, synthetic construct
  REGION 1..123: note = Synthetic Polypeptide
EVQLLESGGGLVQPGRSLRLSCAASGFTFDDYAMHWVRQAPGKGLEWVSGISWNSGRIGYADSVKGRFTISRDNAKNSLYLQMNSLRPEDTAFYYCAKGGSTSARYSSGWYYWGQGTLVTVSS

SEQ ID NO: 146 - protein, 109 aa, synthetic construct
  REGION 1..109: note = Synthetic Polypeptide
QSVLTQPPSVSVSPGQTATITCSGDALPKQYVYWYRQKPGQAPLLVIYEDSERPSGIPERFSGSSSGTEVTLSISGVQAEDEADYYCQSTDSSGTYVVFGGGTKLTVLS

SEQ ID NO: 147 - protein, 116 aa, synthetic construct
  REGION 1..116: note = Synthetic Polypeptide
EVQLVQSGGGLVQPGGSLRLSCLVSGFTFNNYPMHWVRQAPGKGLEWVAVISYDGNNKYYADSVKGRFTISRDNSKNTLYLQMNSLRSEDTAVYYCARGGGGFDYWGQGTLVTVSS

SEQ ID NO: 148 - protein, 105 aa, synthetic construct
  REGION 1..105: note = Synthetic Polypeptide
EIVMTQSPGTLSLSPGERATLSCRASQSVSSSSLAWYQQKPGQAPRLLIYGASSRATGIPDRFSGSGSGTDFTLTISRLEPEDFAVYYCQQYGAFGGGTKVEIKR

SEQ ID NO: 149 - protein, 120 aa, synthetic construct
  REGION 1..120: note = Synthetic Polypeptide
EVQLLESGGGLVQPGGSLRLSCAASGFTFSNYWMSWVRQAPGKGLEWVSSISSSSSYIYYADSVKGRFTISRDNSKNTLYLQMNSLRAEDTAVYYCARQSESSVYGMDVWGQGTLVTVSS

SEQ ID NO: 150 - protein, 113 aa, synthetic construct
  REGION 1..113: note = Synthetic Polypeptide
QSVLTQPPSASGTPGQRVTISCSGSHSNIASSSANWYQQLPGTAPKLLIYGNDQRPSGVPDRFSGSKSGTSASLAISGLRSEDEADYYCAAWDDSLSGVVFGGGTKLTVLGQP

SEQ ID NO: 151 - protein, 122 aa, synthetic construct
  REGION 1..122: note = Synthetic Polypeptide
EVQLLESGGGLVQPGGSLRLSCAASGFTFSNAWMSWVRQAPGKGLEWVSSISSSSSYIYYADSVKGRFTISRDNSKNTLYLQMNSLRAEDTAVYYCARDGSIFGVVTPFDYWGQGTLVTVSS

SEQ ID NO: 152 - protein, 116 aa, synthetic construct
  REGION 1..116: note = Synthetic Polypeptide
QSVLTQPPSASGTPGQRVTISCTGSSSNIGAGYDVHWYQQLPGTAPKLLIYSNNQRPSGVPDRFSGSKSGTSASLAISGLRSEDEADYYCAAWDDSLRGPHWVFGGGTKLTVLGQP

SEQ ID NO: 153 - protein, 117 aa, synthetic construct
  REGION 1..117: note = Synthetic Polypeptide
EVQLLESGGGLVQPGGSLRLSCAASGFTFSNTWMNWVRQAPGKGLEWVSSISVNSNYIYYADSVKGRFTISRDNSKNTLYLQMNSLRAEDTAVYYCARDSEYAFQNWGQGTLVTVSS

SEQ ID NO: 154 - protein, 113 aa, synthetic construct
  REGION 1..113: note = Synthetic Polypeptide
QSVLTQPPSASGTPGQRVTISCTGSSSNIGAGYDVHWYQQLPGTAPKLLIYDNEWRPSGVPDRFSGSKSGTSASLAISGLRSEDEADYYCATWDDSLHWVFGGGTKLTVLGQP

SEQ ID NO: 155 - protein, 121 aa, synthetic construct
  REGION 1..121: note = Synthetic Polypeptide
EVQLLESGGGLVQPGGSLRLSCAASGFTFSDHWMSWVRQAPGKGLEWVSSISSSSSYIYYADSVKGRFTISRDNSKNTLYLQMNSLRAEDTAVYYCARHEDTYYYYGMDVWGQGTLVTVSS

SEQ ID NO: 156 - protein, 113 aa, synthetic construct
  REGION 1..113: note = Synthetic Polypeptide
QSVLTQPPSASGTPGQRVTISCSGSFSNIGGNTVNWYQQLPGTAPKLLIYDNDQRPSGVPDRFSGSKSGTSASLAISGLRSEDEADYYCASWDDSLSGVVFGGGTKLTVLGQP

SEQ ID NO: 157 - protein, 120 aa, synthetic construct
  REGION 1..120: note = Synthetic Polypeptide
EVQLLESGGGLVQPGGSLRLSCAASGFTFRKDWMSWVRQAPGKGLEWVAVISYDGSNKYYADSVKGRFTISRDNSKNTLYLQMNSLRAEDTAVYYCTRAAPHYYYGLDVWGQGTLVTVSS

SEQ ID NO: 158 - protein, 113 aa, synthetic construct
  REGION 1..113: note = Synthetic Polypeptide
QSVLTQPPSASGTPGQRVTISCSGSSSNIGSNTVNWYQQLPGTAPKLLIYYDDLLPSGVPDRFSGSKSGTSASLAISGPRSEDEADYYCGSWDSSLSAVVFGGGTKLTVLGQP

SEQ ID NO: 159 - protein, 121 aa, synthetic construct
  REGION 1..121: note = Synthetic Polypeptide
QVQLVESGGGVVQPGRSLRLSCAASGFTFSGFGMHWVRQAPGKGLEWVAVISYDGRNKYYADSVKGRFTISRDNSKNTLYLQMNSLRAEDTAVYYCAKSLSGTYWGYGMDVWGQGTTVTVS

SEQ ID NO: 160 - protein, 107 aa, synthetic construct
  REGION 1..107: note = Synthetic Polypeptide
DIQMTQSPSTLSASVGDRVTITCRASQSIRSWLAWYQQKPGKAPKLLIYKASSLESGVPSRFSGSGSGTEFTLTISSLQPDDFATYYCQQYNSYLWTFGQGTKVEIK

SEQ ID NO: 161 - protein, 121 aa, synthetic construct
  REGION 1..121: note = Synthetic Polypeptide
QVQLVESGGGVVQPGRSLRLSCAASGFTFSGYGIHWVRQAPDKGLEWVAVISYDGTNKYYGDSVKGRFTISRDNTKNTLYLQMNSLRTEDTAVYYCAKSLSGTYWGYGMDVWGQGTTVTVS

SEQ ID NO: 162 - protein, 107 aa, synthetic construct
  REGION 1..107: note = Synthetic Polypeptide
DIQMTQSPSTLSASVGDRVTITCRASQNIGNWLAWYQQKPGKAPKLLIYKASSLQNGVPSRFSGSGSGTEFTLTISSLQPDDFATYYCQRYNSYSWTFGQGTKVEIK

SEQ ID NO: 163 - protein, 121 aa, synthetic construct
  REGION 1..121: note = Synthetic Polypeptide
QVQLVESGGGVVQPGRSLRLSCAASGFTFSGYGMHWVRQAPGKGLDWVTVISYDGSNKHYADSVKGRFTISRDNSKNTLYLQMSSLGPEDTAVYYCAKSLSGTYWGYGMDVWGQGTTVTVS

SEQ ID NO: 164 - protein, 107 aa, synthetic construct
DIQMTQSPSTLSASVGDRVTITCRASQNISSWLAWYQQKPGKAPKLLIYKASSLESGVPSRFSGSGSGTEFTLTVSSLQPDDFATYYCQQYNRYSWTFGQGTKVEIK

SEQ ID NO: 165 - protein, 121 aa, synthetic construct
QVQLVOSGAEVKKPGSSVKVSCKASGYAFTNYYIEWVRQAPGQGLEWMGVINPGWGDTNYNEKFKGRVTITADKSTSTAYMELSSLRSEDTAVYYCARRDTAHGYYALDPWGQGTTVTVSS

SEQ ID NO: 166 - protein, 107 aa, synthetic construct
DIQMTQSPSSLSASVGDRVTITCKASONVGTNVAWYQQKPGKAPKLLIYSASYRYSGVPSRFSGSGSGTDFTLTISSLQPEDFATYYCQQYWDYPLTFGGGTKVEIK

SEQ ID NO: 167 - protein, 121 aa, synthetic construct
QVQLQQSGAELVRPGTSVKVSCKASGYAFTNYYIEWLKORPGQGLEWIGVINPGWGDTNYNEKFKGKATLTADKSSSTAYMQLSSLTSDDSAVFFCARRDTRHGYYALDYWGQGTSVTVSS

SEQ ID NO: 168 - protein, 107 aa, synthetic construct
DIVMTQSQKFMSTSVGDRVSVTCKASONVGTNVAWYOQKPGQSPKALIYSASYRYSGVPDRFTGSGSGTDFTLTISNVOSEDLAEYFCQQYNDYPLTFGAGTRLEIK

SEQ ID NO: 169 - protein, 105 aa, synthetic construct
  REGION 1..105: note = Synthetic Polypeptide
QPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 170 - protein, 104 aa, synthetic construct
  REGION 1..104: note = Synthetic Polypeptide
QPKSTPTLTVFPPSSEELKENKATLVCLISNFSPSGVTVAWKANGTPITQGVDTSNPTKEGNKFMASSFLHLTSDQWRSHNSFTCQVTHEGDTVEKSLSPAECL

SEQ ID NO: 171 - protein, 107 aa, synthetic construct
  REGION 1..107: note = Synthetic Polypeptide
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 172 - protein, 97 aa, synthetic construct
  REGION 1..97: note = Synthetic Polypeptide
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKI

SEQ ID NO: 173 - protein, 13 aa, synthetic construct
  REGION 1..13: note = Synthetic Polypeptide
VPRDCGCKPCICT

SEQ ID NO: 174 - protein, 214 aa, synthetic construct
  REGION 1..214: note = Synthetic Polypeptide
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 175 - protein, 335 aa, synthetic construct
  REGION 1..335: note = Synthetic Polypeptide
AKTTAPSVYPLVPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPHQRVPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEVLHNHLTTKTISRSLGK

SEQ ID NO: 176 - protein, 335 aa, synthetic construct
  REGION 1..335: note = Synthetic Polypeptide
KTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 177 - protein, 329 aa, synthetic construct
  REGION 1..329: note = Synthetic Polypeptide
TTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 178 - protein, 106 aa, synthetic construct
  REGION 1..106: note = Synthetic Polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 107 aa, synthetic construct [sequence omitted]

SEQ ID NO: 180 - protein, 105 aa, synthetic construct
  REGION 1..105: note = Synthetic Polypeptide
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 181 - protein, 106 aa, synthetic construct
  REGION 1..106: note = Synthetic Polypeptide
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 182 - protein, 98 aa, synthetic construct
  REGION 1..98: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 183 - protein, 12 aa, synthetic construct
  REGION 1..12: note = Synthetic Polypeptide
EPKSCDKTHTCP

SEQ ID NO: 184 - protein, 220 aa, synthetic construct
  REGION 1..220: note = Synthetic Polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 330 aa, synthetic construct [sequence omitted]

SEQ ID NO: 186 - protein, 98 aa, synthetic construct
  REGION 1..98: note = Synthetic Polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 187 - protein, 12 aa, synthetic construct
  REGION 1..12: note = Synthetic Polypeptide
ESKYGPPCPSCP

SEQ ID NO: 188 - protein, 217 aa, synthetic construct
  REGION 1..217: note = Synthetic Polypeptide
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 189 - protein, 327 aa, synthetic construct
  REGION 1..327: note = Synthetic Polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 190 - protein, 98 aa, synthetic construct
  REGION 1..98: note = Synthetic Polypeptide
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 191 - protein, 62 aa, synthetic construct
  REGION 1..62: note = Synthetic Polypeptide
ELKTPLGDTTHTCPRCPEPKSCDTPPPCPRCPEPKSCDTPPPCPRCPEPKSCDTPPPCPRCP

SEQ ID NO: 192 - protein, 216 aa, synthetic construct
  REGION 1..216: note = Synthetic Polypeptide
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 193 - protein, 377 aa, synthetic construct
  REGION 1..377: note = Synthetic Polypeptide
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 194 - protein, 98 aa, synthetic construct
  REGION 1..98: note = Synthetic Polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 195 - protein, 12 aa, synthetic construct
  REGION 1..12: note = Synthetic Polypeptide
ERKCCVECPPCP

SEQ ID NO: 196 - protein, 216 aa, synthetic construct
  REGION 1..216: note = Synthetic Polypeptide
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 197 - protein, 326 aa, synthetic construct
  REGION 1..326: note = Synthetic Polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 198 - protein, 448 aa, synthetic construct
  REGION 1..448: note = Synthetic Polypeptide
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 199 - protein, 215 aa, synthetic construct
  REGION 1..215: note = Synthetic Polypeptide
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 5 aa, synthetic construct
  REGION 1..5: note = Synthetic Polypeptide
SYAMS

SEQ ID NO: 201 - protein, 17 aa, synthetic construct
  REGION 1..17: note = Synthetic Polypeptide
GITGSGGSTYYADSVKG

SEQ ID NO: 202 - protein, 13 aa, synthetic construct
  REGION 1..13: note = Synthetic Polypeptide
DPGTTVIMSWFDP

SEQ ID NO: 203 - protein, 122 aa, synthetic construct
  REGION 1..122: note = Synthetic Polypeptide
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSS

SEQ ID NO: 204 - protein, 326 aa, synthetic construct
  REGION 1..326: note = Synthetic Polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 205 - protein, 12 aa, synthetic construct
  REGION 1..12: note = Synthetic Polypeptide
RASQSVRGRYLA

SEQ ID NO: 206 - protein, 7 aa, synthetic construct
  REGION 1..7: note = Synthetic Polypeptide
GASSRAT

SEQ ID NO: 207 - protein, 9 aa, synthetic construct
  REGION 1..9: note = Synthetic Polypeptide
QQYGSSPRT

SEQ ID NO: 208 - protein, 111 aa, synthetic construct
  REGION 1..111: note = Synthetic Polypeptide
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTV

SEQ ID NO: 209 - protein, 104 aa, synthetic construct
  REGION 1..104: note = Synthetic Polypeptide
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 380 aa, synthetic construct
  REGION 1..380: note = Synthetic Polypeptide
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 211 - protein, 401 aa, synthetic construct
  REGION 1..401: note = Synthetic Polypeptide
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 212 - protein, 317 aa, synthetic construct
  REGION 1..317: note = Synthetic Polypeptide
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 213 - protein, 250 aa, synthetic construct
  REGION 1..250: note = Synthetic Polypeptide
FYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 214 - protein, 178 aa, synthetic construct
  REGION 1..178: note = Synthetic Polypeptide
IRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 215 - protein, 5 aa, synthetic construct
  REGION 1..5: note = Synthetic Polypeptide
DLATE

SEQ ID NO: 216 - protein, 39 aa, synthetic construct
  REGION 1..39: note = Synthetic Polypeptide
GFYYLYANICFRHHETSGDLATEYLQLMVYVTKTSIKIP